(19) United States
(12) Patent Application Publication
(10) Pub. No.: US 2025/0163062 A1
(43) Pub. Date: May 22, 2025

(54) WNT PATHWAY INHIBITOR COMPOUNDS

(71) Applicant: ADLAI NORTYE BIOPHARMA CO., LTD., Hangzhou, Zhejiang (CN)

(72) Inventors: Yufeng CHEN, Hangzhou (CN); Peng WU, Hangzhou (CN); Zhao SUN, Hangzhou (CN); Feifan LI, Hangzhou (CN); Han YANG, Hangzhou (CN); Canfeng LIU, Hangzhou (CN); Meng LV, Hangzhou (CN); Wanli CHENG, Hangzhou (CN); Kaixuan CHEN, Hangzhou (CN); Chaofan JIN, Hangzhou (CN); Keke CHEN, Hangzhou (CN); Youping WANG, Hangzhou (CN); Xiaoli ZHU, Hangzhou (CN); Nanhai HE, Hangzhou (CN)

(73) Assignee: ADLAI NORTYE BIOPHARMA CO., LTD., Hangzhou, Zhejiang (CN)

(21) Appl. No.: 18/834,030

(22) PCT Filed: Jan. 29, 2023
(86) PCT No.: PCT/CN2023/073641

§ 371 Date: Jul. 29, 2024

(30) Foreign Application Priority Data
Jan. 30, 2022 (CN) 202210115506.1

(51) Int. Cl. C07D 471/16; A61K 31/519

(52) U.S. Cl. CPC C07D 471/16; A61K 31/519

(57) ABSTRACT

The present disclosure provides a compound of formula I or a pharmaceutically acceptable salt, isotope derivative, or stereoisomer thereof having excellent Wnt pathway inhibitory activity. The present disclosure also provides a method for preparing the compound and use of the compound in preventing and/or treating cancer, tumor, inflammatory disease, autoimmune disease or immune-mediated disease.

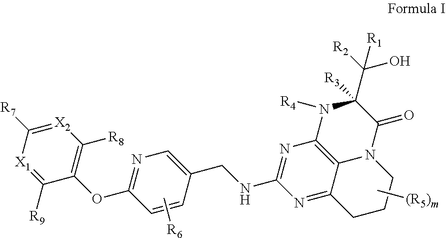

This application claims priority to a Chinese patent application filed with the State Intellectual Property Office of China on Jan. 30, 2022, with application number 202210115506.1 and title “Wnt pathway inhibitor compounds”, all contents of which are incorporated by reference into this application.

### TECHNICAL FIELD

The present disclosure relates to a heterocyclic compound, in particular to a highly active Wnt pathway inhibitor and application thereof.

### BACKGROUND

The Wnt/β-catenin signaling pathway is a conserved pathway in biological evolution. In normal somatic cells, β-catenin only acts as a cytoskeletal protein and forms a complex with E-cadherin at the cell membrane to maintain the adhesion of homotypic cells and prevent cell movement. When the Wnt signaling pathway is not activated, β-catenin in the cytoplasm is phosphorylated and forms a β-catenin degradation complex with APC, Axin and GSK3β, thereby initiating the ubiquitin system to degrade s-catenin via the proteasome pathway, maintaining the β-catenin level in the cytoplasm at a low level. When cells are stimulated by Wnt signals. Wnt proteins bind to the specific receptor Frizzled protein on the cell membrane. The activated Frizzled receptor recruits intracellular Dishevelled protein, inhibits the degradation activity of the β-catenin degradation complex formed by GSK3β and other proteins, and stabilizes the free β-catenin protein in the cytoplasm. After stably accumulated in the cytoplasm. β-catenin enters the cell nucleus and binds to the LEF/TCF transcription factor family to initiate the transcription of downstream target genes (such as c-myc, c-jun, Cyclin D1, etc). Overactivation of the Wnt/β-catenin signaling pathway is closehy related to the occurrence of various cancers (including colon cancer, gastric cancer, breast cancer, etc.). For example, abnormal activation of the Wnt classical signaling pathway and nuclear accumulation of β-catenin protein are common in colorectal cancer, and inhibiting the activity of the Wnt signaling pathway can inhibit the proliferation of cancers such as colon cancer. APC mutations are found in more than 85% of colorectal cancers. The mutated APC blocks the phosphorylation and degradation of β-catenin, inducing the occurrence of colorectal cancer. In addition. Axin mutations and β-catenin mutations can also cause intracellular aggregation of β-catenin and activate the Wnt/β-catenin pathway.

Although it is known that inhibition of the Wnt signaling pathway can effectively prevent and/or treat cancer, tumors, inflammatory diseases, autoimmune diseases and immune-mediated diseases, there is currently a lack of satisfactory and effective Wnt pathway inhibitor compounds in the prior art. Therefore, it is necessary to develop effective Wnt pathway inhibitor compounds.

### SUMMARY OF THE DISCLOSURE

One aspect of the present disclosure provides a class of compounds having a structure of Formula I or a pharmaceutically acceptable salt, isotopic derivative, or stereoisomer thereof:

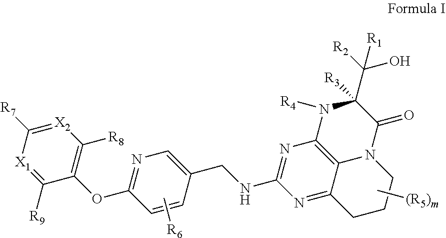

wherein, X₁ and X₂ are both N, or one of them is N and the other is CH;
R₁ and R₂ each independently represent hydrogen, halogen, (C₁-C₆) alkyl, or halogenated (C₁-C₆) alkyl;
R₃ represents hydrogen, halogen, (C₁-C₆) alkyl, halogenated (C₁-C₆) alkyl;
R₄ represents hydrogen, halogen, (C₁-C₆) alkyl, halogenated (C₁-C₆) alkyl, or R₄ and R₂ form a 4-8 membered ring;
R₅ each independently represents hydrogen, halogen, (C₁-C₆) alkyl, halogenated (C₁-C₆) alkyl, (C₃-C₈) cycloalkyl, halogenated (C₃-C₈) cycloalkyl, or two R₅ connected to the same carbon atom form a 3-5 membered ring; m is 0, 1, 2 or 3;
R₆ each independently represents hydrogen, halogen, —CN, (C₁-C₆) alkyl, halogenated (C₁-C₆) alkyl;
R₇ represents hydrogen, halogen, (C₁-C₆) alkyl, halogenated (C₁-C₆) alkyl, (C₃-C₈) cycloalkyl, halogenated (C₃-C₈) cycloalkyl, —ORₐ, -halogenated ORₐ, —SRₐ, -halogenated SRₐ;
R₈ represents hydrogen, —(C₁-C₆)alkyl, -halogenated (C₁-C₆)alkyl; R₉ represents halogen, (C₁-C₆) alkyl, halogenated (C₁-C₆) alkyl, (C₃-C₈) cycloalkyl, halogenated (C₃-C₈) cycloalkyl, —(C₁-C₆) alkylene CN, -halogenated (C₁-C₆) alkylene CN, —(C₃-C₈) cycloalkylene CN, -halogenated (C₃-C₈) cycloalkylene CN, —NRₐRₐ′, —(C₁-C₆) alkylene NRₐRₐ′, -halogenated (C₁-C₆) alkylene NRₐRₐ′, wherein Rₐ, Rₐ′ can form a 4-8 membered ring with the N to which they are connecte;
Rₐ and Rₐ′ each independently represent hydrogen, (C₁-C₆) alkyl, halogenated (C₁-C₆) alkyl, (C₃-C₈) cycloalkyl or halogenated (C₃-C₈) cycloalkyl.

In one embodiment of the present disclosure, R₄ is selected from (C₁-C₆)alkyl, halogenated (C₁-C₆)alkyl.

In one embodiment of the present disclosure, R₅ is selected from hydrogen, halogen, (C₁-C₆)alkyl, halogenated (C₁-C₆)alkyl.

In one embodiment of the present disclosure, R₆ is hydrogen.

In one embodiment of the present disclosure, X₁ and X₂ are both N, and R₉ is selected from (C₁-C₆) alkyl, halogenated (C₁-C₆) alkyl, (C₃-C₈) cycloalkyl, and halogenated (C₃-C₈) cycloalkyl.

In one embodiment of the present disclosure, when X₁ and X₂ are both N, R₇ is not halogen.

In one embodiment of the present disclosure, R₇ is selected from (C₁-C₆)alkyl, halogenated (C₁-C₆)alkyl.

In one embodiment of the present disclosure, R₅ is halogen.

In one embodiment of the present disclosure, R₅ is fluorine.

In one embodiment of the present disclosure, when X₁ and X₂ are both N, R₉ represents halogen, (C₁-C₆) alkyl, halogenated (C₁-C₆) alkyl, (C₃-C₈) cycloalkyl, halogenated (C₃-C₈) cycloalkyl, —(C₁-C₆) alkylene CN, -halogenated (C₁-C₆) alkylene CN, —(C₃-C₈) cycloalkylene CN, -halogenated (C₃-C₈) cycloalkylene CN, —NRₐRₐ′, —(C₁-C₆) alkylene NRₐRₐ′, -halogenated (C₁-C₆) alkylene NRₐRₐ′, wherein Rₐ, Rₐ′ can form a 4-8 membered ring with the N to which they are connected.

In one embodiment of the present disclosure, when one of X₁ and X₂ is N and the other is CH, R₉ represents halogen, —(C₁-C₆)alkylene CN, -halogenated (C₁-C₆)alkylene CN, —(C₃-C₈)cycloalkylene CN, -halogenated (C₃-C₈)cycloalkylene CN, —NRₐRₐ′, —(C₁-C₆)alkylene NRₐRₐ′, -halogenated (C₁-C₆)alkylene NRₐRₐ′, wherein Rₐ, Rₐ′ can form a 4-8 membered ring with the N to which they are connected.

In one embodiment of the present disclosure, when one of X₁ and X₂ is N and the other is CH, R₉ represents (C₁-C₆) alkyl, halogenated (C₁-C₆) alkyl, (C₃-C₈) cycloalkyl, halogenated (C₃-C₈) cycloalkyl.

In another aspect of the present disclosure, there is also provided a compound having the following structure or a pharmaceutically acceptable salt, isotopic derivative, stereoisomer thereof, wherein the compound has the following structure:

| Serial | |
|---|---|
| number | Compound structure |
| 1 | 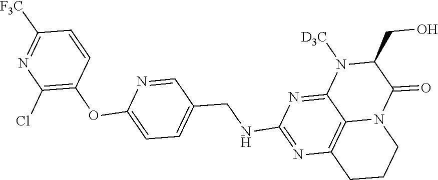 [SMILES: [2H]C([2H])([2H])N1c2nc(NCc3ccc(Oc4ccc(C(F)(F)F)nc4Cl)nc3)nc3c2N(CCC3)C(=O)[C@@H]1CO] |
| 2 | 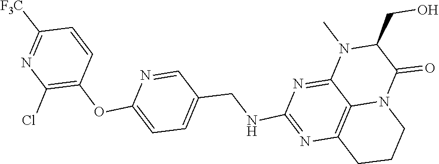 [SMILES: CN1c2nc(NCc3ccc(Oc4ccc(C(F)(F)F)nc4Cl)nc3)nc3c2N(CCC3)C(=O)[C@@H]1CO] |
| 3 | 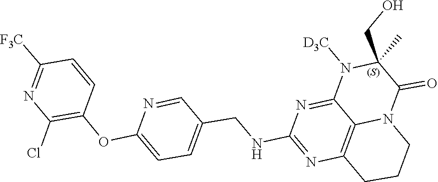 |
| 4 | 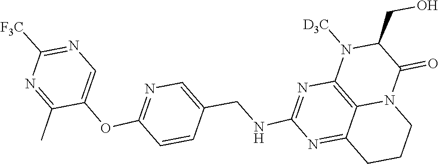 [SMILES: [2H]C([2H])([2H])N1c2nc(NCc3ccc(Oc4cnc(C(F)(F)F)nc4C)nc3)nc3c2N(CCC3)C(=O)[C@@H]1CO] |
| 5 | 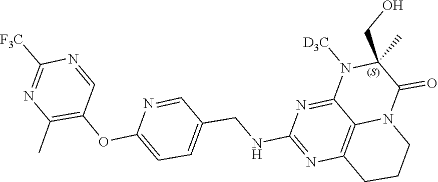 |
| 6 | 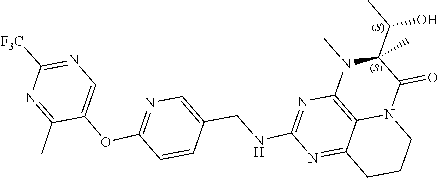 |
| 7 | 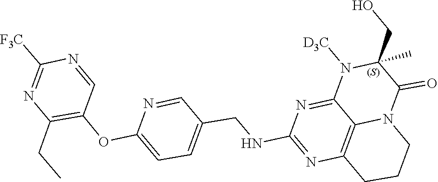 |
| 8 | 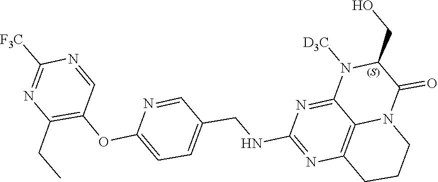 |
| 9 | 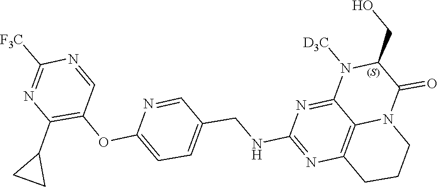 |
| 10 | 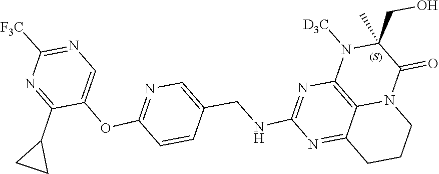 |
| 11 | 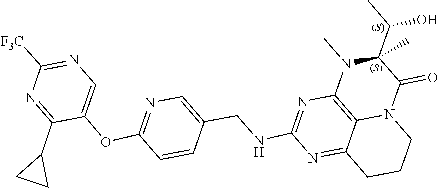 |
| 12 | 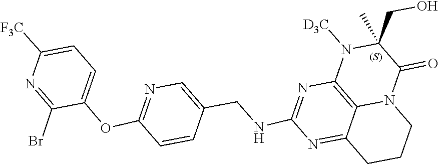 |
| 13 | 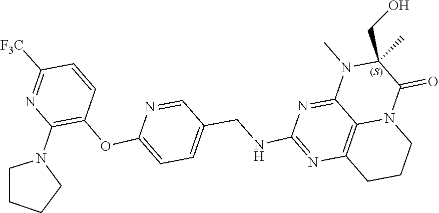 |
| 14 | 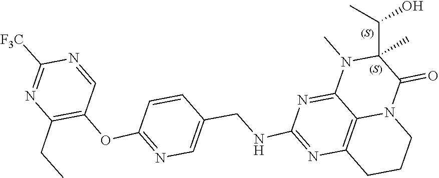 |
| 15 | 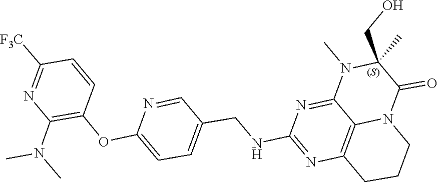 |
| 16 | 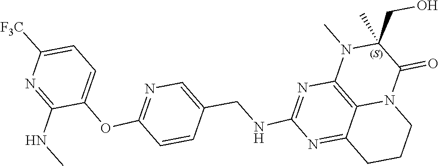 |
| 17 | 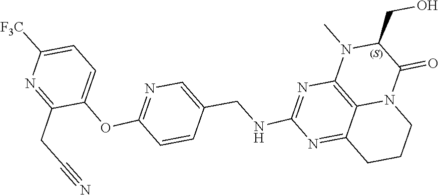 |
| 18 | 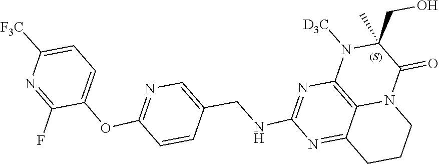 |
| 19 | 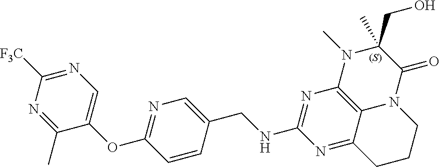 |
| 20 | 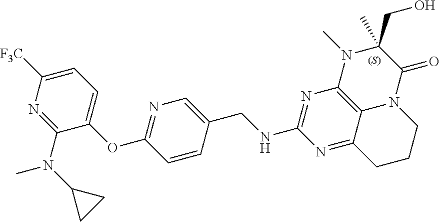 |
| 21 | 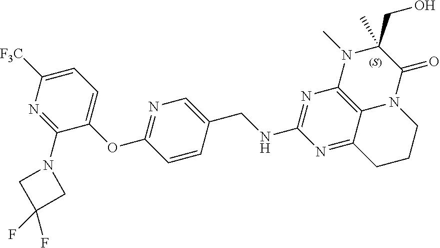 |
| 22 | 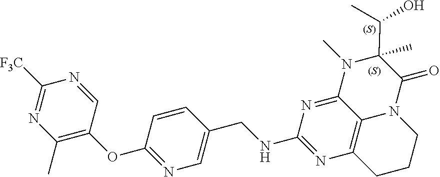 |
| 23 | 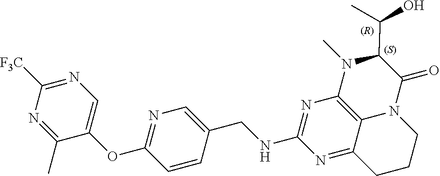 |
| 24 | 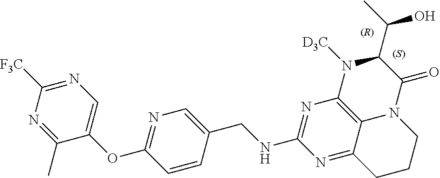 |
| 25 | 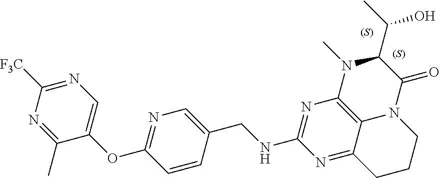 |
| 26 | 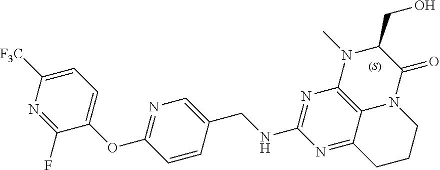 |
| 27 | 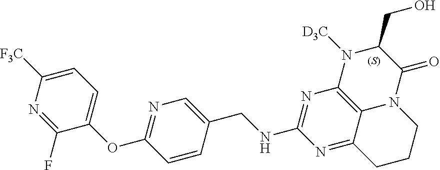 |
| 28 | 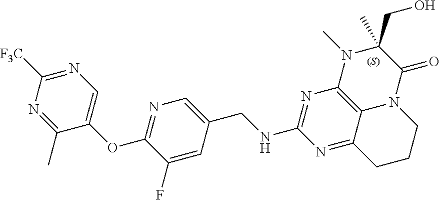 |
| 29 | 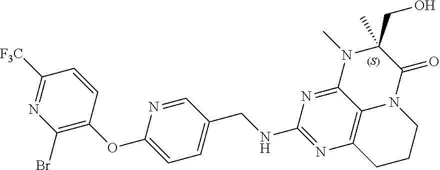 |
| 30 | 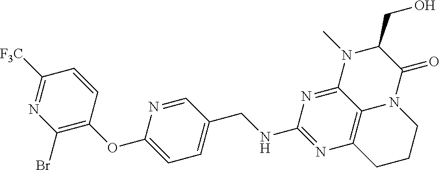 [SMILES: CN1c2nc(NCc3ccc(Oc4ccc(C(F)(F)F)nc4Br)nc3)nc3c2N(CCC3)C(=O)[C@@H]1CO] |
| 31 | 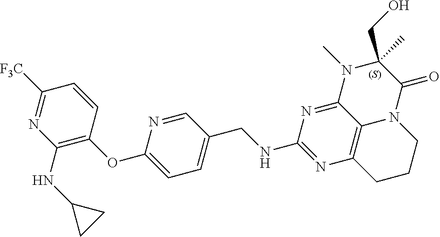 |
| 32 | 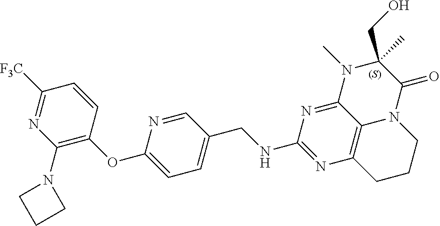 |
| 33 | 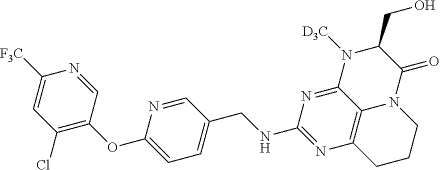 [SMILES: [2H]C([2H])([2H])N1c2nc(NCc3ccc(Oc4cnc(C(F)(F)F)cc4Cl)nc3)nc3c2N(CCC3)C(=O)[C@@H]1CO] |
| 34 | 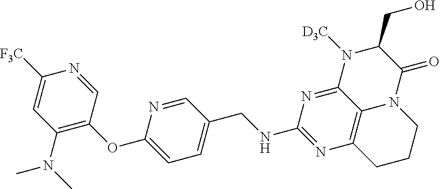 [SMILES: [2H]C([2H])([2H])N1c2nc(NCc3ccc(Oc4cnc(C(F)(F)F)cc4N(C)C)nc3)nc3c2N(CCC3)C(=O)[C@@H]1CO] |
| 35 | 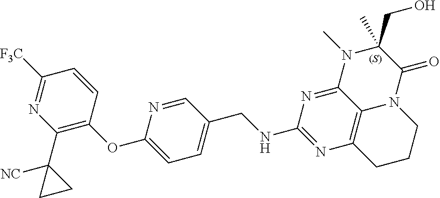 |
| 36 | 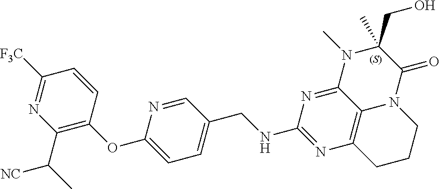 |
| 37 | 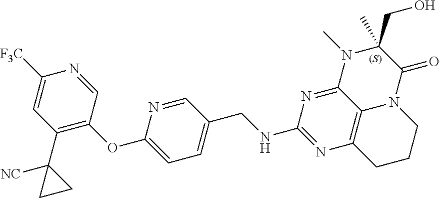 |
| 38 | 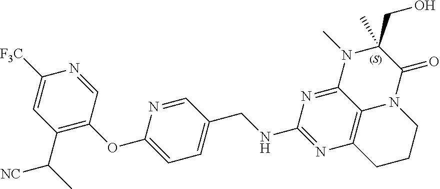 |
| 39 | 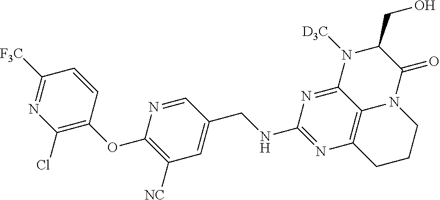 [SMILES: [2H]C([2H])([2H])N1c2nc(NCc3cnc(Oc4ccc(C(F)(F)F)nc4Cl)c(C#N)c3)nc3c2N(CCC3)C(=O)[C@@H]1CO] |
| 40 | 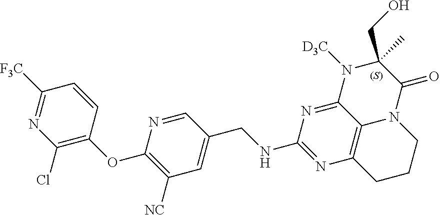 |
| 41 | 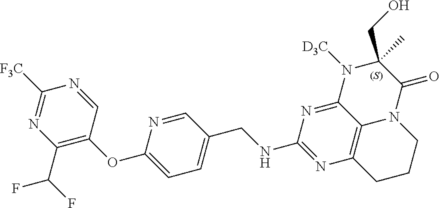 |
| 42 | 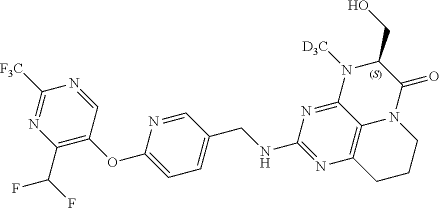 |
| 43 | 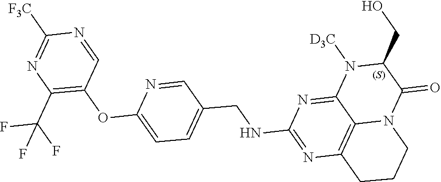 |
| 44 | 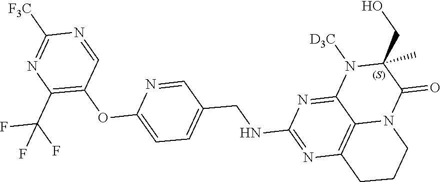 |
| 45 | 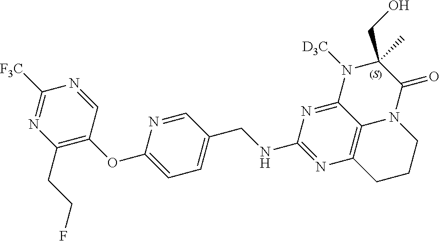 |
| 46 | 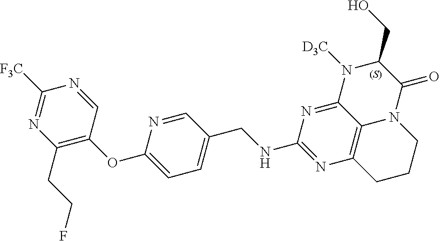 |
| 47 | 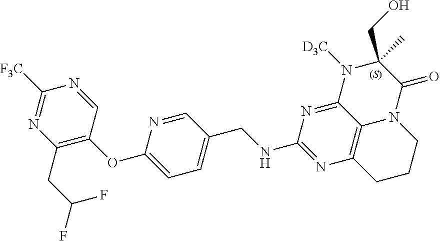 |
| 48 | 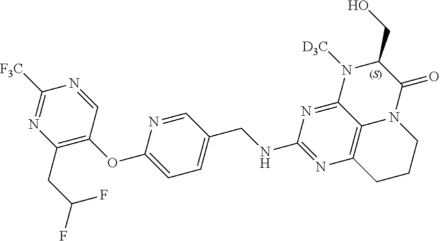 |
| 49 | 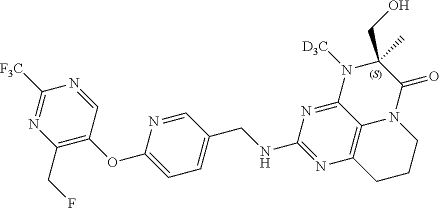 |
| 50 | 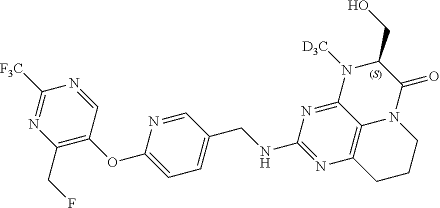 |
| 51 | 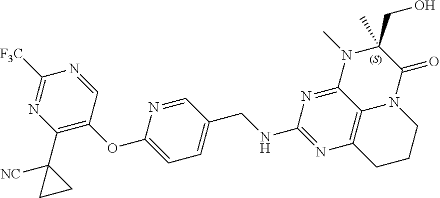 |
| 52 | 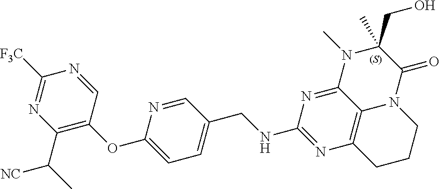 |
| 53 | 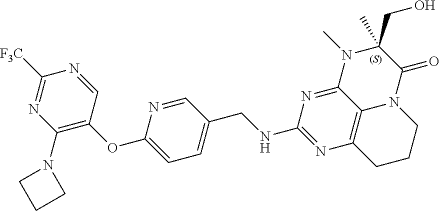 |
| 54 | 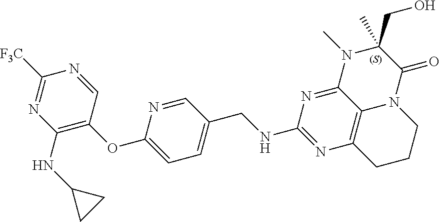 |
| 55 | 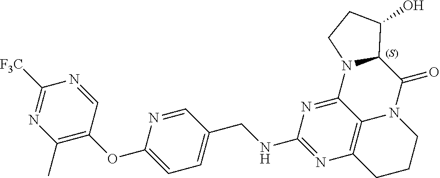 |
| 56 | 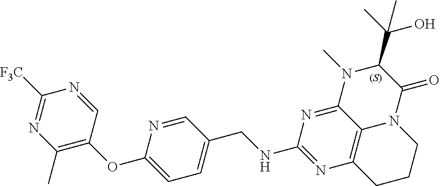 |
| 57 | 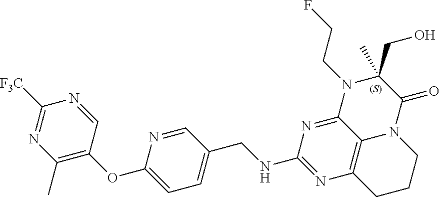 |
| 58 | 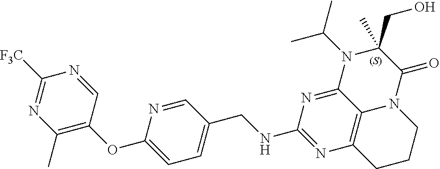 |
| 59 | 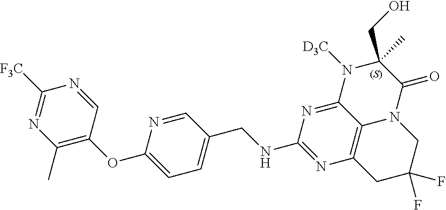 |
| 60 | 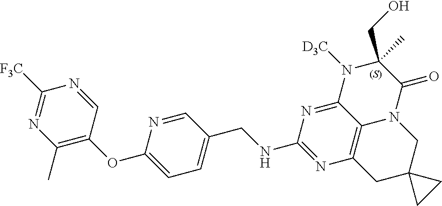 |
| 61 | 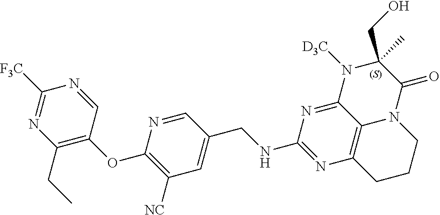 |
| 62 | 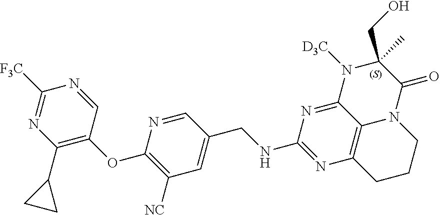 |
| 63 | 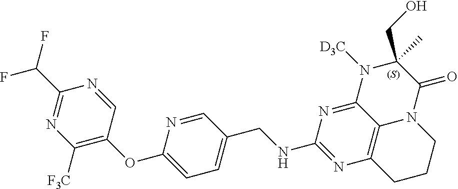 |
| 64 | 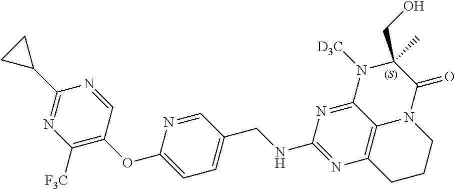 |
| 65 | 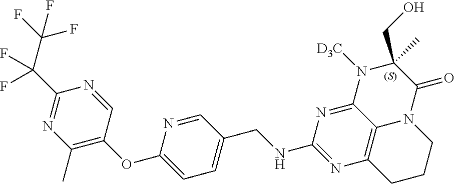 |
| 66 | 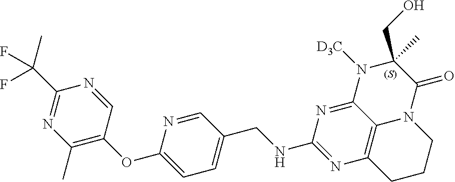 |
| 67 | 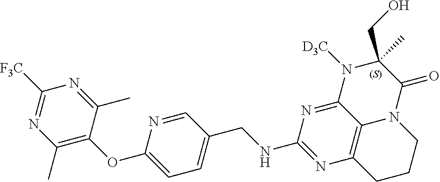 |
| 68 | 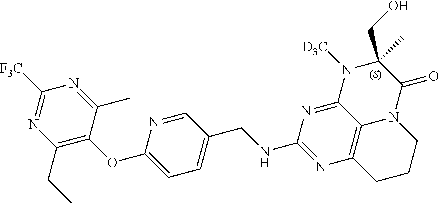 |
| 69 | 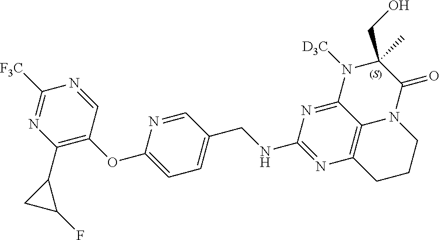 |
| 70 | 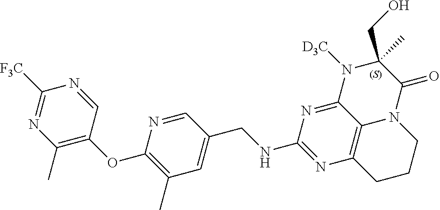 |
| 71 | 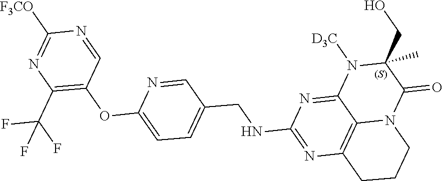 |
| 72 | 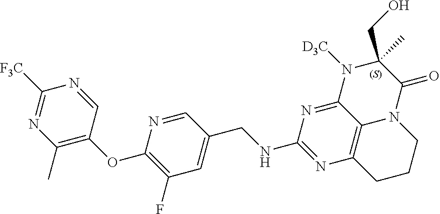 |
| 73 | 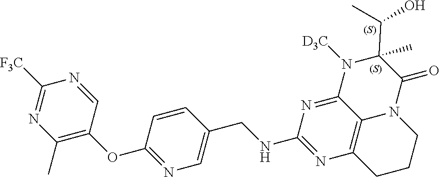 |
| 74 | 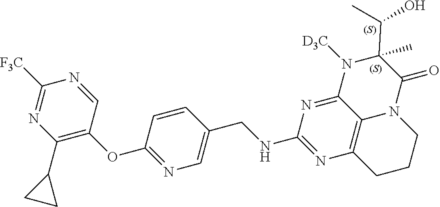 |
| 75 | 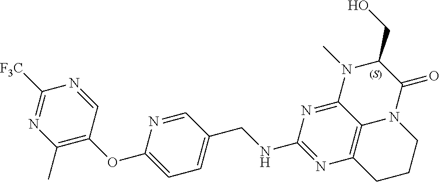 |
| 76 | 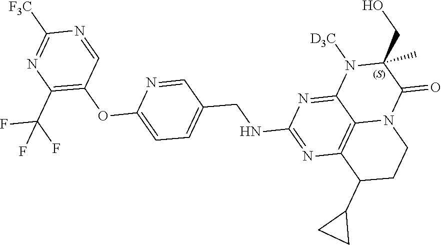 |
| 77 | 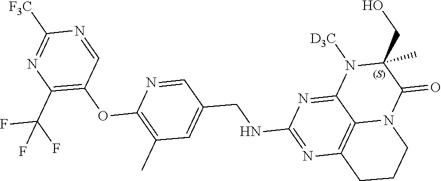 |
| 78 | 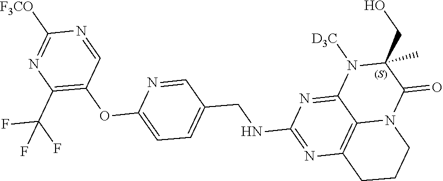 |
| 79 | 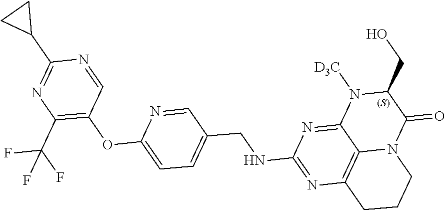 |

In another aspect of the present disclosure, a pharmaceutical composition is provided, comprising any of the aforementioned compounds or pharmaceutically acceptable salts, isotopic derivatives, stereoisomers thereof, and an optional pharmaceutically acceptable carrier.

In another aspect of the present disclosure, there is also provided the use of the aforementioned compound or its pharmaceutically acceptable salt, isotope derivative, stereoisomer or the aforementioned pharmaceutical composition in the preparation of a medicament for preventing and/or treating cancer, tumor, inflammatory disease, autoimmune disease or immune-mediated disease.

It is particularly noted that, in this article, when referring to the “compound” of formula I structure, it generally also covers its stereoisomers, diastereomers, enantiomers, racemic mixtures and isotopic derivatives.

It is well known to those skilled in the art that a salt, solvate, and hydrate of a compound are alternative forms of existence of the compound, and they can all be converted into the compound under certain conditions. Therefore, it is particularly noted that when referring to the compound of formula I structure in this article, it generally also includes its pharmaceutically acceptable salts, and further includes its solvates and hydrates.

Similarly, when referring to a compound in this article, it generally also includes its prodrugs, metabolites and nitrogen oxides.

The pharmaceutically acceptable salt of the present disclosure may be formed using the an inorganic acid or an organic acid, the “pharmaceutically acceptable salt” means a salt that is, within the scope of sound medical judgment, suitable for use in contact with the tissues of humans and lower animals without undue toxicity, irritation, and allergic response, commensurate with a reasonable benefit/risk ratio. As outlined below, the salts may be prepared in situ during the final isolation and purification of the compounds of the present disclosure, or prepared by reacting the free base or free acid with a suitable reagent separately. For example, the free base may be reacted with a suitable acid. In addition, when the compounds of the present disclosure carry an acidic moiety, suitable pharmaceutically acceptable salts thereof may include metal salts, such as alkali metal salts (e.g., sodium or potassium salts); and alkaline earth metal salts (e.g., calcium or magnesium salts). Examples of pharmaceutically acceptable non-toxic acid addition salts are salts formed by amino groups with inorganic acids (e.g., hydrochloric acid, hydrobromic acid, phosphoric acid, sulfuric acid, and perchloric acid) or organic acids (e.g., acetic acid, oxalic acid, maleic acid, tartaric acid, citric acid, succinic acid, or malonic acid), or formed by using other methods known in the prior art such as ion exchange. Other pharmaceutically acceptable salts include adipate, sodium alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfonate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectate, persulfate, 3-phenylpropionate, phosphate, picrate, pivalate, propionate, stearate, succinate, sulfate, tartrate, thiocyanate, p-toluenesulfonate, undecanoate, and valerate. Representative alkali metal or alkaline earth metal salts include salts of sodium, lithium, potassium, calcium, and magnesium. Other pharmaceutically acceptable salts include, nontoxic ammonium salts (where appropriate), quaternary ammonium salts, and ammonium cations formed with counterions, for example, halides, hydroxides, carboxylates, sulfates, phosphates, nitrates, lower alkyl sulfonates, and aryl sulfonates.

The pharmaceutically acceptable salts of the present disclosure may be prepared by a conventional method, for example, by dissolving the compound of the present disclosure in a water-miscible organic solvent (e.g., acetone, methanol, ethanol, and acetonitrile), adding an excess of an aqueous organic or inorganic acid thereto to precipitate the salt from the resulting mixture, removing the solvent and remaining free acid therefrom, and then isolating the precipitated salt.

The precursors or metabolites of the present disclosure may be those known in the art as long as the precursors or metabolites are converted into compounds by metabolism in vivo. For example, “prodrugs” refer to those of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. The term “prodrugs” refer to compounds which yield the parent compounds of the above-mentioned formulae rapidly through transformation in vivo, for example, through metabolism in vivo, or N-demethylation of a compound of the present disclosure.

“Solvate” as used herein means a physical association of a compound of the present disclosure with one or more solvent molecules, whether organic or inorganic. This physical association includes hydrogen bonding. In some cases, for example, when one or more solvent molecules are incorporated into the crystal lattice of a crystalline solid, solvates will be able to be isolated. Solvent molecules in solvates may exist in regular and/or disordered arrangements. Solvates may contain stoichiometric or non-stoichiometric amounts of solvent molecules. “Solvate” encompasses both solution-phase and isolatable solvates. Exemplary solvates include, but are not limited to, hydrates, ethanolates, methanolates, and isopropanolates. Solvation methods are well known in the art.

The “stereoisomerism” described in the present disclosure is divided into conformational isomerism and configurational isomerism, and configurational isomerism can also be divided into cis-trans isomerism and optical isomerism (that is, optical isomerism). Due to the rotation or twisting of carbon and carbon single bonds in organic molecules of a certain configuration, a stereoisomerism phenomenon in which each atom or atomic group of the molecule has a different arrangement in space, the common structures are alkanes and cycloalkanes. Such as the chair conformation and boat conformation that appear in the structure of cyclohexane. “Stereoisomer” means when a compound of the present disclosure contains one or more asymmetric centers and is thus available as racemates and racemic mixtures, single enantiomers, diastereomeric mixtures and single diastereomers. The compound of the present disclosure has an asymmetric center, and each asymmetric center can produce two optical isomers, and the scope of the present disclosure includes all possible optical isomers and diastereoisomer mixtures and pure or partially pure compounds. The compounds described herein may exist in tautomeric forms having different points of attachment of hydrogens by displacement of one or more double bonds. For example, a ketone and its enol form are keto-enol tautomers. Each tautomer and mixtures thereof are included in the compounds of the present disclosure. Enantiomers, diastereoisomers, racemates, mesoisomers, cis-trans isomers, tautomers, geometric isomers, epimers of all compounds of formula (I) Conformants and their mixtures, etc., are included in the scope of the present disclosure.

The “isotopic derivatives” of the present disclosure refer to molecules that are labeled with isotopes of the compounds in this patent. Isotopes commonly used for isotopic labeling are: hydrogen isotopes, ²H and ³H; carbon isotopes: ¹¹C, ¹³C and ¹⁴C; chlorine isotopes: ³⁵Cl and ³⁷Cl; fluorine isotopes: ¹⁸F; iodine isotopes: ¹²³I and ¹²⁵I; nitrogen isotopes: ¹³N and ¹⁵N; Oxygen isotopes: ¹⁵O, ¹⁷O and ¹⁸O and sulfur isotope ³⁵S. These isotope-labeled compounds can be used to study the distribution of pharmaceutical molecules in tissues. Especially deuterium ³H and carbon ¹³C are more widely used due to their easy labeling and convenient detection. Substitution of certain heavy isotopes, such as deuterium (²H), can enhance metabolic stability, prolong half-life and thus provide therapeutic advantages for dose reduction. Isotopically labeled compounds are generally synthesized starting from labeled starting materials and carried out in the same way as non-isotopically labeled compounds using known synthetic techniques.

The present disclosure also provides the use of the compound of the present disclosure in the preparation of a drug for preventing and/or treating cancer, tumors, inflammatory diseases, autoimmune diseases or immune-mediated diseases.

In addition, the present disclosure provides a pharmaceutical composition for preventing and/or treating cancer, tumors, inflammatory diseases, autoimmune diseases, neurodegenerative diseases, attention-related diseases or immune-mediated diseases, which comprises the compound of the present disclosure as an active ingredient. The pharmaceutical composition may optionally contain a pharmaceutically acceptable carrier.

In addition, the present disclosure provides a method for preventing and/or treating cancer, tumors, inflammatory diseases, autoimmune diseases, neurodegenerative diseases, attention-related diseases or immune-mediated diseases, which comprises administering a compound of the present disclosure of formula I structure or a pharmaceutically acceptable salt, isotopic derivative, stereoisomer or pharmaceutical composition of the present disclosure to a mammal in need thereof.

Representative examples of inflammatory, autoimmune, and immune-mediated diseases may include, but are not limited to, arthritis, rheumatoid arthritis, spondyloarthritis, gouty arthritis, osteoarthritis, juvenile arthritis, Other Arthritis Conditions, Lupus, Systemic Lupus Erythematosus (SLE), Skin Related Disorders, Psoriasis, Eczema, Dermatitis, Atopic Dermatitis, Pain, Pulmonary Disease, Lung Inflammation, Adult Respiratory Distress Syndrome (ARDS), pulmonary sarcoidosis, chronic pulmonary inflammatory disease, chronic obstructive pulmonary disease (COPD), cardiovascular disease, atherosclerosis, myocardial infarction, congestive heart failure, myocardial ischemia-reperfusion injury, inflammatory bowel disease, Crohn's disease, ulcerative colitis, irritable bowel syndrome, asthma, Sjogren's syndrome, autoimmune thyroid disease, urticaria (rubella), multiple sclerosis, scleroderma, organ transplant rejection, xenograft, idiopathic thrombocytopenic purpura (ITP), Parkinson's disease, Alzheimer's disease, diabetes-related diseases, inflammation, pelvic inflammatory disease, allergic rhinitis, allergic bronchitis, allergic sinusitis, leukemia, lymphoma, B Cell Lymphoma, T Cell Lymphoma, Myeloma, Acute Lymphocytic Leukemia (ALL), Chronic Lymphocytic Leukemia (CLL), Acute Myeloid Leukemia (AML), Chronic Myelogenous Leukemia (CML), Hairy Cell Leukemia, He Jie King's disease, non-Hodgkin's lymphoma, multiple myeloma, myelodysplastic syndrome (MDS), myeloproliferative neoplasm (MPN), diffuse large B-cell lymphoma, and follicular lymphoma.

Representative examples of cancer or tumor may include, but are not limited to, skin cancer, bladder cancer, ovarian cancer, breast cancer, stomach cancer, pancreatic cancer, prostate cancer, colon cancer, lung cancer, bone cancer, brain cancer, neuroblastoma, rectal cancer, colon cancer, familial adenomatous polyposis carcinoma, hereditary nonpolyposis colorectal cancer, esophagus cancer, lip cancer, larynx cancer, hypopharyngeal cancer, tongue cancer, salivary gland cancer, stomach cancer, adenocarcinoma, medullary thyroid cancer, Papillary thyroid cancer, renal cancer, renal parenchymal cancer, ovarian cancer, cervical cancer, uterine body cancer, endometrial cancer, choriocarcinoma, pancreatic cancer, prostate cancer, testicular cancer, urinary cancer, melanoma, brain tumors such as Glioblastoma, astrocytoma, meningioma, medulloblastoma and peripheral neuroectodermal tumor, Hodgkin lymphoma, non-Hodgkin lymphoma, Burkitt lymphoma, acute lymphoblastic leukemia (ALL), chronic lymphocytic leukemia (CLL), acute myeloid leukemia (AML), chronic myelogenous leukemia (CML), adult T-cell leukemia lymphoma, diffuse large B-cell lymphoma (DLBCL), hepatocellular carcinoma, gallbladder Carcinoma, bronchial carcinoma, small cell lung cancer, non-small cell lung cancer, multiple myeloma, basal cell tumor, teratoma, retinoblastoma, choroidal melanoma, seminoma, rhabdomyosarcoma, craniopharyngioma, osteosarcoma, chondrosarcoma, sarcoma, liposarcoma, fibrosarcoma, Ewing sarcoma, or plasmacytoma.

When the compound of the present disclosure or a pharmaceutically acceptable salt thereof is administered in combination with another anticancer agent or immune checkpoint inhibitor for the treatment of cancer or tumors, the compound of the present disclosure or a pharmaceutically acceptable salt thereof can provide enhanced anticancer effects.

Representative examples of anticancer agents useful in the treatment of cancer or tumors may include, but are not limited to, cell signal transduction inhibitors, chlorambucil, melphalan, cyclophosphamide, ifosfamide, busulfan, carbamate, Mustin, lomustine, streptozotocin, cisplatin, carboplatin, oxaliplatin, dacarbazine, temozolomide, procarbazine, methotrexate, fluorouracil, cytarabine, gemcitabine, Mercaptopurine, fludarabine, vinblastine, vincristine, vinorelbine, paclitaxel, docetaxel, topotecan, irinotecan, etoposide, trabectedin, dactinomycin, doxorubicin, epirubicin, daunorubicin, mitoxantrone, bleomycin, mitomycin C, ixabepilone, tamoxifen, flutamide, gonadorelin analogs, methadone Progesterone, prednisone, dexamethasone, methylprednisolone, thalidomide, interferon alpha, leucovorin, sirolimus, sirolimus ester, everolimus, afatinib, alisertib, amuvatinib, apatinib, axitinib, bortezomib, bosutinib, britinib, cabozantinib, cediranib, crenolanib, kezhuotinib, dabrafenib, dabrafenib, Cotinib, danucitinib, dasatinib, dovitinib, erlotinib, foretinib, ganetespib, gefitinib, ibrutinib, icotinib, imatinib, iniparib, la Patinib, lenvatinib, linifanib, linsitinib, masitinib, momelotinib, motisanib, neratinib, nilotinib, niraparib, oprozomib, olaparib, pazopanib, pictilisib, ponatinib, quizartinib, regorafenib, rigosertib, rucaparib, ruxolitinib, saracatinib, saridegib, sorafenib, sunitinib, tiratinib, tivantinib, tivozanib, tofacitinib, Trametinib, vandetanib, veliparib, vemurafenib, vimodegib, volasertib, alemtuzumab, bevacizumab, berentuzumab vedotin, catumaxumab Antibodies, cetuximab, denosumab, gemtuzumab, ipilimumab, nimotuzumab, ofatumumab, panitumumab, rituximab, tositumumab Monoclonal antibody, trastuzumab, PI3K inhibitor, CSF1R inhibitor, A2A and/or A2B receptor antagonist, IDO inhibitor, anti-PD-1 antibody, anti-PD-L1 antibody, LAG3 antibody, TIM-3 antibody and an anti-CTLA-4 antibody or any combination thereof.

When the compounds of the present disclosure or their pharmaceutically acceptable salts are administered in combination with another therapeutic agent for treating inflammatory diseases, autoimmune diseases and immune-mediated diseases, the compounds of the present disclosure, or pharmaceutically acceptable salts thereof, provide enhanced therapeutic effect.

Representative examples of therapeutic agents useful in the treatment of inflammatory, autoimmune, and immune-mediated diseases can include, but are not limited to, steroidal agents (e.g., prednisone, prednisone, prednisone, methylphenidate, cortisone, cortisone, hydroxycortisone, betamethasone, dexamethasone, etc.), methotrexate, leflunomide, anti-TNFα agents (e.g., etanercept, infliximab, adalib monoclonal antibody, etc.), calcineurin inhibitors (eg, tacrolimus, pimecrolimus, etc.), and antihistamines (eg, diphenhydramine, hydroxyzine, loratadine, ebazan Tin, ketotifen, cetirizine, levocetirizine, fexofenadine, etc.), and at least one therapeutic agent selected from them can be included in the pharmaceutical composition of the present disclosure.

The compounds of the present disclosure or their pharmaceutically acceptable salts can be administered as active ingredients. The dosage of the active ingredient can be adjusted according to a number of relevant factors, such as the condition of the subject to be treated, the type and severity of the disease, the rate of administration and the doctor's opinion. In some cases, an amount less than the above dose may be appropriate. An amount greater than the above dose may be used if no harmful side effects are caused and the amount may be administered in divided doses per day.

In addition, the present disclosure also provides a method for preventing and/or treating tumors, cancers, viral infections, organ transplant rejection, neurodegenerative diseases, attention-related diseases or autoimmune diseases, which comprises administering a compound of the present disclosure or a pharmaceutical composition of the present disclosure to a mammal in need thereof.

The pharmaceutical composition of the present disclosure can be formulated into dosage forms for oral administration or parenteral administration (including intramuscular, intravenous and subcutaneous routes, intratumoral injection) according to any of the conventional methods, such as tablets, granules, powders, capsules, syrups, emulsions, microemulsions, solutions or suspensions.

Pharmaceutical compositions of the present disclosure for oral administration can be prepared by mixing the active ingredient with carriers such as cellulose, calcium silicate, corn starch, lactose, sucrose, dextrose, calcium phosphate, stearic acid, magnesium stearate, calcium stearate, gelatin, talc, surfactant, suspending agent, emulsifier and diluent. Examples of carriers employed in the injectable compositions of the present disclosure are water, saline solution, glucose solution, glucose-like solution, alcohol, glycol, ether (e.g., polyethylene glycol 400), oil, fatty acids, fatty acid esters, glycerides, surfactants, suspending and emulsifying agents.

Other features of the disclosure will become apparent in the course of the description of exemplary embodiments of the disclosure which are given to illustrate the disclosure and are not intended to be limiting thereof, the following examples were prepared, separated and characterized using the methods disclosed in the disclosure. The compounds of the present disclosure can be prepared in a variety of ways known to those skilled in the art of organic synthesis, using the methods described below as well as synthetic methods known in the art of synthetic organic chemistry or by variations thereof known to those skilled in the art to synthesize compounds of the disclosure. Preferred methods include, but are not limited to, those described below. Reactions are performed in solvents or solvent mixtures appropriate to the kit materials used and to the transformations effected. Those skilled in the art of organic synthesis will appreciate that the functionality present on the molecule is consistent with the proposed transitions. This sometimes requires judgment to alter the order of synthetic steps or starting materials to obtain the desired compound of the disclosure.

### Embodiment

### Term

Terms used in the present disclosure, including the specification and claims, are defined as follows, unless otherwise indicated. It must be noted that, in the description and the appended claims, the singular forms “a”, “an”, and “the” include plural referents unless the context clearly dictates otherwise. If not stated otherwise, conventional methods of mass spectrometry, nuclear magnetic, HPLC, protein chemistry, biochemistry, recombinant DNA techniques and pharmacology are used. In this use, the use of “or” or “and” means “and/or” if not stated otherwise.

Throughout the specification and claims, a given chemical formula or name shall encompass all stereo and optical isomers and racemates in which such isomers exist. Unless otherwise indicated, all chiral (enantiome and diastereoisomer) and racemic forms are within the scope of the present disclosure. Many geometric isomers of C═C double bonds, C═N double bonds, and ring systems may also be present in the compounds, and all the above-mentioned stable isomers are encompassed in the present disclosure. Cis- and trans- (or E- and Z-) geometric isomers of the compounds of the present disclosure are described herein and may be isolated as mixtures of isomers or as separated isomeric forms. The compounds of the present disclosure may be isolated in optically active or racemic forms. All methods for preparing the compounds of the present disclosure and intermediates prepared therein are considered part of the present disclosure. In preparing enantiomeric or diastereomeric products, they can be isolated by conventional methods, for example, by chromatography or fractional crystallization. Depending on the process conditions, the final products of the present disclosure are obtained in free (neutral) or salt form.

Both the free forms and salts of these end products are within the scope of the present disclosure. If desired, one form of the compound may be converted to another form.

The free base or acid may be converted to a salt; the salt may be converted to the free compound or another salt; mixtures of isomeric compounds of the present disclosure may be isolated into the individual isomers. The compounds, free forms and salts thereof of the present disclosure, may exist in a variety of tautomeric forms in which hydrogen atoms are transposed onto other parts of the molecule and the chemical bonds between the atoms of the molecule are thus rearranged. It is to be understood that all tautomeric forms which may exist are included in the present disclosure.

Unless otherwise defined, the definitions of substituents of the present disclosure are each independent and not interrelated, e.g., for R^{a} (or R^{a′}) in substituents, they are each independent in the definition of different substituents. Specifically, when a definition of R^{a} (or R^{a′}) is selected in a substituent, it does not mean that Rₐ (or R^{a′}) has the same definition in other substituents. More specifically, for example (a non-exhaustive list) for NR^{a}R^{a′}, when the definition of R^{a} (or R^{a′}) is selected from hydrogen, it does not mean that in —C(O)—NR^{a}R^{a′}, Rₐ (or R^{a′}) must be hydrogen. In another aspect, when there are more than one Rₐ (or R^{a′}) in a substituent, these Rₐ (or R^{a′}) are also independent of each other. For example, in the substituent —(CR^{a}R^{a′})m-O—(CR^{a}R^{a})n-, when m+n is greater than or equal to 2, the m+n Rₐ (or R^{a′}) are independent of each other, and they may have the same or different meanings.

Unless otherwise defined, when a substituent is labeled “optionally substituted”, the substituent is selected from, for example, the following substituents consisting of alkyl, cycloalkyl, aryl, heterocyclyl, halogen, hydroxy, alkoxy, oxo, alkanoyl, aryloxy, alkanoyloxy, amino, alkylamino, arylamino, arylalkylamino, disubstituted amine group (in which two amino substituents are selected from alkyl, aryl or arylalkyl), alkanoylamino, aroylamino, aralkanoylamino, substituted alkanoylamino, substituted arylamino, substituted aralkanoylamino, thio, alkylthio, arylthio, arylalkylthio, arylthiocarbonyl, arylalkylthiocarbonyl, alkylsulfonyl, arylsulfonyl, arylalkylsulfonyl, sulfonamido such as —SO₂NH₂, substituted sulfonamido, nitro, cyano, carboxy, carbamoyl such as —CONH2, substituted carbamoyl such as —CONH alkyl, —CONH aryl, —CONH arylalkyl or the case where there are two substituents selected from alkyl, aryl or arylalkyl on the nitrogen, alkoxycarbonyl, aryl, substituted aryl, guanidino, heterocyclyl such as indolyl, imidazolyl, furanyl, thienyl, thiazolyl, pyrrolidinyl, pyridyl, pyrimidinyl, pyrrolidinyl, piperidinyl, morpholinyl, piperazinyl, and homopiperazinyl, and substituted heterocyclyl.

As used herein, the term “alkyl” or “alkylene” is intended to include both branched and straight chain saturated aliphatic hydrocarbon groups having the specified number of carbon atoms. For example, “C₁-C₆ alkyl” denotes an alkyl group having 1 to 6 carbon atoms. Examples of alkyl groups include, but are not limited to, methyl (Me), ethyl (Et), n-propyl, isopropyl, n-butyl, isobutyl, tert-butyl, n-pentyl, isopentyl, and neopentyl. As used herein, the alkyl is preferably an alkyl having 1 to 6, 1 to 4, and more preferably 1 to 3 carbon atoms.

The term “alkenyl” denotes a straight or branched chain hydrocarbon group containing one or more double bonds and typically 2 to 20 carbon atoms in length.

For example, “C2-C6 alkenyl” contains 2 to 6 carbon atoms. Alkenyl groups include, but are not limited to, for example, ethenyl, propenyl, butenyl, and 1-methyl-2-buten-1-yl.

The term “alkynyl” denotes a straight or branched chain hydrocarbon group containing one or more triple bonds and typically 2 to 20 carbon atoms in length. For example, “C2-C6 alkynyl” contains 2 to 6 carbon atoms. Representative alkynyl groups include, but are not limited to, for example, ethynyl, 1-propynyl, and 1-butynyl.

The term “alkoxy” or “alkyloxy” refers to —O-alkyl. “C1-C6 alkoxy” (or alkyloxy) is intended to include C1, C2, C3, C4, C5, and C6 alkoxy. Examples of alkoxy groups include, but are not limited to, methoxy, ethoxy, propoxy (e.g., n-propoxy and isopropoxy), and t-butoxy. Similarly, “alkylthio” or “thioalkoxy” means an alkyl group, as defined above, with the specified number of carbon atoms linked via a sulfur bridge; for example, methyl-S— and ethyl-S—.

The term “carbonyl” refers to an organic functional group (C═O) composed of two carbon and oxygen atoms linked by a double bond.

The term “aryl”, alone or as part of a larger moiety such as “aralkyl”, “aralkoxy”, or “aryloxyalkyl”, refers to a monocyclic, bicyclic, or tricyclic ring system having a total of 5 to 12 ring members, where at least one ring in the system is aromatic and where each ring in the system contains 3 to 7 ring members. In certain embodiments of the present disclosure, “aryl” refers to an aromatic ring system including, but not limited to, phenyl, biphenyl, indanyl, 1-naphthyl, 2-naphthyl, and tetrahydronaphthyl. The term “aralkyl” or “arylalkyl” refers to an alkyl residue attached to an aryl ring. Non-limiting examples include benzyl, and phenethyl. The fused aryl group may be attached to another group at a suitable position on the cycloalkyl ring or the aromatic ring. For example, a dashed line drawn from a ring system indicates that the bond may be attached to any suitable ring atom.

The term “cycloalkyl” refers to a monocyclic or bicyclic alkyl group. Monocyclic alkyl refers to C₃-C₈ cyclic alkyl including, but not limited to, cyclopropyl, cyclobutyl, cyclopentyl, cyclohexyl, and norbornyl. Branched cycloalkyl such as 1-methylcyclopropyl and 2-methylcyclopropyl are included in the definition of “cycloalkyl”. Bicyclic alkyl includes bridged, spiro, or fused cycloalkyl.

The term “cycloalkenyl” refers to a monocyclic or bicyclic alkenyl group. Monocyclic alkenyl refers to C₃-C₈ cyclic alkenyl including, but not limited to, cyclopropenyl, cyclobutenyl, cyclopentenyl, cyclohexenyl, and norbomenyl. Branched cycloalkenyl such as 1-methylcyclopropenyl and 2-methylcyclopropenyl are included in the definition of “cycloalkenyl”. Bicyclic alkenyl includes bridged, spiro or fused cyclic alkenyl. “Halogenated” or “halogen” includes fluoro, chloro, bromo and iodo. “Haloalkyl” is intended to include both branched and straight-chain saturated aliphatic hydrocarbon groups having the specified number of carbon atoms and substituted with one or more halogens. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl, and heptachloropropyl. Examples of haloalkyl also include “fluoroalkyl” groups intended to include branched and straight chain saturated aliphatic hydrocarbon groups having the specified number of carbon atoms and substituted with one or more fluorine atoms.

“Haloalkoxy” or “haloalkyloxy” denotes a haloalkyl group, as defined above, having the indicated number of carbon atoms linked via an oxygen bridge. For example, “C1-C6 haloalkoxy” is intended to include C1, C2, C3, C4, C5, and C6 haloalkoxy.

Examples of haloalkoxy include, but are not limited to, trifluoromethoxy, 2,2,2-trifluoroethoxy, and pentafluoroethoxy. Similarly, “haloalkylthio” or “thiohaloalkoxy” denotes a haloalkyl group, as defined above, having the indicated number of carbon atoms linked via a sulfur bridge; for example, trifluoromethyl-S- and pentafluoroethyl-S—.

The -halogenated (C1-C6) alkylene CN, -halo (C3-C8) cycloalkylene CN, -halo (C1-C6) alkylene NR Rₐ Rₐ′, etc. described herein mean —(C1-C6) alkylene CN, —(C3-C8) cycloalkylene CN, —(C1-C6) alkylene NR Rₐ Rₐ′, etc., which are optionally halogenated.

In the present disclosure, the expression Cx1-Cx2 is used when referring to some substituent groups, which means that the number of carbon atoms in the substituent group may be x1 to x2. For example, C0-C8 means that the group contains 0, 1, 2, 3, 4, 5, 6, 7 or 8 carbon atoms, C1-C8 means that the group contains 1, 2, 3, 4, 5, 6, 7 or 8 carbon atoms, C2-C8 means that the group contains 2, 3, 4, 5, 6, 7 or 8 carbon atoms, C3-C8 means that the group contains 3, 4, 5, 6, 7 or 8 carbon atoms, C4-C8 means that the group contains 4, 5, 6, 7 or 8 carbon atoms, C0-C6 means that the group contains 0, 1, 2, 3, 4, 5 or 6 carbon atoms, C1-C6 means that the group contains 1, 2, 3, 4, 5 or 6 carbon atoms, C2-C6 means that the group contains 2, 3, 4, 5 or 6 carbon atoms, and C₃-C₆ means that the group contains 3, 4, 5 or 6 carbon atoms.

In the present disclosure, the expression “x1-x2 membered ring” is used when referring to cyclic groups such as aryl, heteroaryl, cycloalkyl and heterocycloalkyl, which means that the number of ring atoms of the group may be x1 to x2. For example, the 3- to 12-membered cyclic group may be a 3, 4, 5, 6, 7, 8, 9, 10, 11, or 12 membered ring, the number of ring atoms of which may be 3, 4, 5, 6, 7, 8, 9, 10, 11, or 12; the 3- to 6-membered ring means that the cyclic group may be a 3, 4, 5 or 6 membered ring, the number of ring atoms of which may be 3, 4, 5 or 6; the 3- to 8-membered ring means that the cyclic group may be a 3, 4, 5, 6, 7 or 8 membered ring, the number of ring atoms of which may be 3, 4, 5, 6, 7 or 8; the 3- to 9-membered ring means that the cyclic group may be a 3, 4, 5, 6, 7, 8 or 9 membered ring, the number of ring atoms of which may be 3, 4, 5, 6, 7, 8 or 9; the 4- to 7-membered ring means that the cyclic group may be a 4, 5, 6 or 7 membered ring, the number of ring atoms of which may be 4, 5, 6 or 7; the 5- to 8-membered ring means that the cyclic group may be a 5, 6, 7 or 8 membered ring, the number of ring atoms of which may be 5, 6, 7 or 8; the 5- to 12-membered ring means that the cyclic group may be a 5, 6, 7, 8, 9, 10, 11 or 12 membered ring, the number of ring atoms of which may be 5, 6, 7, 8, 9, 10, 11 or 12; and the 6- to 12-membered ring means that the cyclic group may be a 6, 7, 8, 9, 10, 11 or 12 membered ring, the number of ring atoms of which may be 6, 7, 8, 9, 10, 11 or 12. The ring atom may be a carbon atom or a heteroatom, for example, a heteroatom selected from N, O and S. When the ring is a heterocycle, the heterocycle may contain 1, 2, 3, 4, 5, 6, 7, 8, 9, 10 or more ring heteroatoms, for example, a heteroatom selected from N, O and S.

In the present invention, one or more halogens may each independently be selected from fluorine, chlorine, bromine, and iodine.

The term “heteroaryl” means a stable 3-, 4-, 5-, 6-, or 7-membered aromatic monocyclic or aromatic bicyclic or 7-, 8-, 9-, 10-, 11-, 12-membered aromatic polycyclic heterocycle, which is fully unsaturated, partially unsaturated, and contains carbon atoms and 1, 2, 3 or 4 heteroatoms independently selected from N, O and S; and includes any polycyclic group in which any heterocycle defined above is fused to a benzene ring. The nitrogen and sulfur heteroatoms may optionally be oxidized. The nitrogen atom is substituted or unsubstituted (i.e., N or NR, where R is H or another substituent if defined). The heterocycle may be attached to its pendant group at any heteroatom or carbon atom that results in a stable structure. If the resulting compound is stable, the heterocyclyl groups described herein may be substituted on a carbon or nitrogen atom. The nitrogen in the heterocycle may be optionally quaternized.

Preferably, when the total number of S and O atoms in the heterocycle exceeds 1, then these heteroatoms are not adjacent to each other. Preferably, the total number of S and O atoms in the heterocycle is not greater than 1. When the term “heterocycle” is used, it is intended to include heteroaryl. Examples of heteroaryls include, but are not limited to, acridinyl, azetidinyl, azocinyl, benzimidazolyl, benzofuranyl, benzothiofuranyl, benzothienyl, benzoxazolyl, benzoxazolinyl, benzothiazolyl, benzotriazolyl, benzotetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuranyl, furanyl, furazanyl, imidazolidinyl, imidazolinyl, imidazolyl, 1H-indazolyl, imidazopyridinyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isothiazolopyridinyl, isoxazolyl, isoxazolopyridinyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridinyl, oxazolidinyl, perimidinyl, oxindolyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolopyridinyl, pyrazolyl, pyridazinyl, pyridooxazolyl, pyridoimidazolyl, pyridothiazolyl, pyridinyl, pyrimidinyl, pyrrolidinyl, pyrrolinyl, 2-pyrrolidonyl, 2H-pyrrolyl, pyrrolyl, quinazolinyl, quinolinyl, 4H-quinolizinyl, quinoxalinyl, quinuclidinyl, tetrazolyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thiazolopyridyl, thienothiazolyl, thienooxazolyl, thienoimidazolyl, thienyl, triazinyl, 1,2,3-triazolyl, 1,2,4-triazolyl, 1,2,5-triazolyl, 1,3,4-triazolyl and xanthenyl, quinolinyl, isoquinolinyl, phthalazinyl, quinazolinyl, indolyl, isoindolyl, indolinyl, 1H-indazolyl, benzimidazolyl, 1,2,3,4-tetrahydroquinolinyl, 1,2,3,4-tetrahydroisoquinolinyl, 5,6,7,8-tetrahydro-quinolinyl, 2,3-dihydro-benzofuranyl, chromanyl, 1,2,3,4-tetrahydro-quinoxalinyl and 1,2,3,4-tetrahydro-quinazolinyl. The term “heteroaryl” may also include biaryl structures formed from “aryl” and monocyclic “heteroaryl” as defined above, for example, but not limited to “-phenylbipyridyl-”, “-phenylbipyrimidinyl”, “-pyridylbiphenyl”, “-pyridylbipyrimidinyl-” “-pyrimidinylbiphenyl-”; where the present disclosure also includes fused and spiro compounds containing, for example, the above-mentioned heterocycles.

As used herein, the term “heterocycloalkyl” refers to a monocyclic heterocycloalkyl system, or a bicyclic heterocycloalkyl system, and also includes spiroheterocycles or bridged heterocycloalkyl groups. The monocyclic heterocycloalkyl refers to a saturated or unsaturated but not aromatic 3- to 8-membered cyclic alkyl system containing at least one heteroatom selected from O, N, S, or P. The bicyclic heterocycloalkyl system refers to a heterocycloalkyl fused with a phenyl, or a cycloalkyl, or a cycloalkenyl, or a heterocycloalkyl, or a heteroaryl.

As used herein, the term “bridged cycloalkyl” refers to polycyclic compounds that share two or more carbon atoms, including bicyclic bridged cyclic hydrocarbons and polycyclic bridged cyclic hydrocarbons. The former are composed of two alicyclic rings sharing more than two carbon atoms; the latter are a bridged cyclic hydrocarbons consisting of more than three rings.

As used herein, the term “spirocycloalkyl” refers to polycyclic hydrocarbons that share one carbon atom (referred to as a spiro atom) between single rings.

As used herein, the term “bridged cycloheteryl” refers to polycyclic compounds that share two or more carbon atoms, and contain at least one atom selected from O, N, or S. including bicyclic bridged heterocycles and polycyclic bridged heterocycles.

As used herein, the term “heterospirocyclyl” refers to polycyclic hydrocarbons that share one carbon atom (referred to as a spiro atom) between single rings, and contain at least one heteroatom selected from O, N, or S.

As used herein, the term “substituted” means that at least one hydrogen atom is replaced with a non-hydrogen group, provided that normal valency is maintained and that the substitution results in a stable compound. As used herein, the ring double bond is a double bond (e.g., C═C, C═N, or N═N) formed between two adjacent ring atoms.

In the case where nitrogen atoms (e.g., amines) are present on the compounds of the present disclosure, these nitrogen atoms may be converted to N-oxides by treatment with an oxidizing agent (e.g., mCPBA and/or hydrogen peroxide) to obtain other compounds of the present disclosure. Thus, the nitrogen atoms shown and claimed are considered to encompass both the nitrogen shown and its N-oxides to obtain the derivatives of the present disclosure.

When any variable occurs more than once in any composition or formula of a compound, its definition at each occurrence is independent of its definition at every other occurrence. Thus, for example, if a group is shown to be substituted with 0-3 R, the group may be optionally substituted with up to three R groups, and at each occurrence R is independently selected from the definition of R. Furthermore, combinations of substituents and/or variables are permissible only if such combinations result in stable compounds.

As used herein, the term “patient” refers to an organism treated by the methods of the present disclosure. Such organisms preferably include, but are not limited to, mammals (e.g., murine, ape/monkey, equine, bovine, swine, canine, feline, etc.) and most preferably refer to humans.

As used herein, the term “effective amount” means an amount of a drug or pharmaceutical agent (i.e., a compound of the present disclosure) that will elicit the biological or medical response of a tissue, system, animal or human that is being sought, for example, by a researcher or clinician. Furthermore, the term “therapeutically effective amount” means an amount results in an improved treatment, cure, prevention or alleviation of a disease, condition or side effect, or a reduction in the rate of progression of a disease or condition, as compared to a corresponding subject not receiving such an amount. An effective amount can be administered in one or more dosing, administrations, or dosages and is not intended to be limited by the particular formulation or route of administration. The term also includes an amount effective that enhances normal physiological function within its scope.

The term “treatment” as used herein includes any effect leading to improvement of a condition, disease, disorder, etc., such as alleviation, reduction, regulation, improvement or elimination, or improvement of its symptoms.

The term “pharmaceutical” or “pharmaceutically acceptable” is used herein to refer to those compounds, materials, compositions, and/or dosage forms as follows: within the scope of sound medical judgment, suitable for use in contact with the tissues of humans and animals without undue toxicity, irritation, allergic response, and/or other problems or complications, commensurate with a reasonable benefit/risk ratio. As used herein, the phrase “pharmaceutically acceptable carrier” means a pharmaceutical material, composition or vehicle, such as a liquid or solid filler, diluent, excipient, manufacturing adjuvant (e.g., lubricant, talc, magnesium stearate, calcium stearate or zinc stearate or stearic acid), or solvent encapsulating material, which refers to carrying or transporting the subject compound from one organ or portion of the body to another organ or portion of the body. Each carrier must be “acceptable” in the sense of being compatible with the other ingredients of the formulation and not deleterious to the patient.

The term “pharmaceutical composition” means a composition including a compound of the present disclosure and optionally other pharmaceutically acceptable carrier. “Pharmaceutically acceptable carrier” means a medium generally accepted in the art for the delivery of a biologically active agent to an animal, particularly a mammal, and includes, i.e., adjuvants, excipients, or vehicles such as diluents, preservatives, fillers, flow regulating agents, disintegrating agents, wetting agents, emulsifying agents, suspending agents, sweetening agents, flavoring agents, perfuming agents, antibacterial agents, antifungal agents, lubricating agents, and dispersing agents. This depends on the mode of administration and the nature of the dosage form.

### Certain Pharmaceutical and Medical Terms

The term “acceptable”, as used herein, means that a formulation ingredient or active ingredient does not have an undue adverse effect on health for the general purpose of treatment.

The term “cancer”, as used herein, refers to an abnormal growth of cells that cannot be controlled and, under certain conditions, is capable of metastasizing (spreading). Cancers of this type include, but are not limited to, solid tumors (e.g., bladder, bowel, brain, chest, uterus, heart, kidney, lung, lymphoid tissue (lymphoma), ovary, pancreas or other endocrine organs (eg, thyroid), prostate, skin (melanoma), or blood cancer (such as non-leukemic leukemia).

The term “administration in combination” or similar terms, as used herein, refers to the administration of several selected therapeutic agents to a patient, in the same or different modes of administration at the same or different times.

The term “enhancing” or “capable of enhancing”, as used herein, means that the desired result can be increased or prolonged, either in potency or duration. Thus, in relation to enhancing the therapeutic effect of a drug, the term “capable of potentiating” refers to the ability of the drug to increase or prolong its potency or duration in the system. As used herein, “potency value” refers to the ability to maximize the enhancement of another therapeutic drug in an ideal system.

The term “immune disease” refers to a disease or condition of an adverse or deleterious reaction to an endogenous or exogenous antigen. The result is usually dysfunction of the cells, or destruction thereof and dysfunction, or destruction of organs or tissues that may produce immune symptoms.

The terms “kit” and “product packaging” are synonymous.

The term “subject” or “patient” includes mammals and non-mammals. Mammals include, but are not limited to, mammals: humans, non-human primates such as orangutans, apes, and monkeys; agricultural animals such as cattle, horses, goats, sheep, and pigs; domestic animals such as rabbits and dogs; experimental animals include rodents, such as rats, mice and guinea pigs. Non-mammalian animals include, but are not limited to, birds, fish, and the like. In a preferred embodiment, the selected mammal is a human.

The term “treatment”, “course of treatment” or “therapy” as used herein includes alleviating, suppressing or improving the symptoms or conditions of a disease; inhibiting the development of complications; improving or preventing the underlying metabolic syndrome; inhibiting the development of diseases or symptoms, Such as controlling the development of a disease or condition; alleviating a disease or a symptom; causing a disease or a symptom to regress; alleviating a complication caused by a disease or a symptom, or preventing and/or treating a sign caused by a disease or a symptom.

As used herein, a certain compound or pharmaceutical composition, after administration, can improve a certain disease, symptom or situation, especially improve its severity, delay the onset, slow down the progression of the disease, or reduce the duration of the disease. Circumstances that may be attributable to or related to the administration, whether fixed or episodic, continuous or intermittent.

### Route of Administration

Suitable routes of administration include, but are not limited to, oral, intravenous, rectal, aerosol, parenteral, ocular, pulmonary, transdermal, vaginal, ear canal, nasal administration and topical administration. In addition, by way of example only, parenteral administration includes intramuscular injection, subcutaneous injection, intravenous injection, intramedullary injection, intraventricular injection, intraperitoneal injection, intralymphatic injection, and intranasal injection.

In one aspect, the compounds described herein are administered locally rather than systemically. In certain embodiments, the depot formulation is administered by implantation (e.g., subcutaneously or intramuscularly) or by intramuscular injection. Additionally, in another embodiment, the drug is administered via a targeted drug delivery system. For example, liposomes coated with organ-specific antibodies. In such embodiments, the liposomes are selectively directed to and taken up by specific organs.

### EXAMPLE

### General Process

When the preparation route is not included, the raw materials and reagents used in the present disclosure are known products, which can be synthesized according to methods known in the art, or can be obtained by purchasing commercially available products. All commercially available reagents were used without further purification. Room temperature means 20-30° C.

If here is no special description in the reaction examples, the reactions are all carried out under a nitrogen atmosphere. The nitrogen atmosphere refers to a nitrogen balloon of about 1 L connected to the reaction flask.

The hydrogenation reaction is usually vacuumized and filled with hydrogen, and the operation is repeated 3 times. The hydrogen atmosphere means that the reaction bottle is connected with a hydrogen balloon of about 1 L.

Microwave reactions use the Biotage® Initiator⁺ Microwave Reactor.

The structures of the compounds of the present disclosure were determined by nuclear magnetic resonance (NMR) and mass spectroscopy (MS). NMR shifts (δ) are given in units of 10⁻⁶ (ppm). The determination of NMR is to use (Bruker Ascend™ 500 type) nuclear magnetic analyzer, the measurement solvent is deuterated dimethyl sulfoxide (DMSO-d6), deuterated chloroform (CDCl3), deuterated methanol (CD30D), and the internal standard is tetramethylsilane (TMS). The following abbreviations are used for the multiplicity of NMR signals: s=singlet, brs=broad, d=doublet, t=triplet, m=multiplet. Coupling constants are listed as J values, measured in Hz.

For LC-MS determination, a Thermo liquid mass spectrometer (UltiMate 3000+MSQ PLUS) was used. For HPLC measurement, a Thermo high pressure liquid chromatograph (UltiMate 3000) was used. For Reverse-Phase Preparative Chromatography a Thermo (UltiMate 3000) reverse-phase preparative chromatograph was used. The flash column chromatography uses Agela (FS-9200T) automatic column passing machine, and the silica gel prepacked column uses Santai SEPAFLASH® prepacked column. Yantai Huanghai HSGF254 or Qingdao GF254 silica gel plates are used for thin-layer chromatography silica gel plates, and the specifications of thin-layer chromatography separation and purification products are 0.4 mm to 0.5 mm.

The synthetic methods of some intermediates in the present disclosure are as follows:

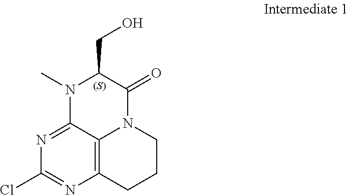

Intermediate 1 was prepared by the following steps:

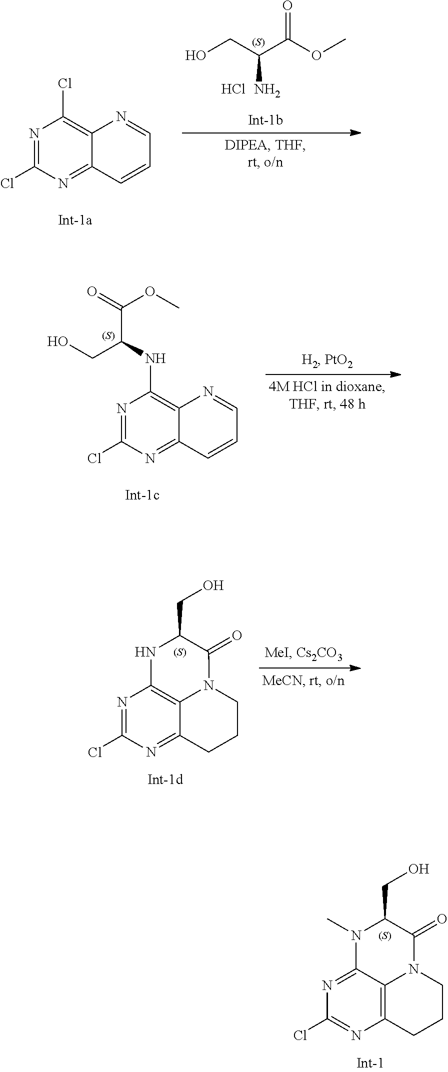

Step 1: Dissolve compound Int-1a (5.0 g, 25.00 mmol), Int-1b (3.87 g, 32.50 mmol) and N, N-diisopropylethylamine (9.69 g, 74.99 mmol) in tetrahydrofuran (50 mL) and react at room temperature overnight. After the reaction was completed as monitored by LCMS, water (100 mL) was added, the mixture was filtered, the filter cake was washed with water, and then dried to obtain a white solid Int-1c (5.5 g, yield 77%). ESI-MS (m/z): 283.2 [M+H]⁺.

Step 2: Dissolve compound Int-1c (5.5 g, 19.46 mmol), platinum dioxide (441 mg, 1.95 mmol) and hydrochloric acid-1,4-dioxane solution (4.86 mL, 19.46 mmol, 4M) in tetrahydrofuran (50 mL), replace with hydrogen balloon three times and react at room temperature for 48 hours. After the reaction was completed as monitored by LCMS, methanol (100 mL) was added for dilution, the mixture was filtered, the filter cake was washed with methanol, the filtrate was alkalized with ammonia methanol (7M) (pH=9-10), then filtered, the filter cake was washed with water, and then dried to obtain a gray solid Int-1d (3.0 g, yield 60%). ESI-MS (m/z): 255.2 [M+H]⁺.

Step 3: Add compound Int-1d (1.0 g, 3.93 mmol) and cesium carbonate (2.56 g, 7.85 mmol) into a 100 mL single-necked flask, add acetonitrile (20 mL), then add iodomethane (585 mg, 4.12 mmol) dropwise, and react at room temperature overnight.

After the reaction was completed as monitored by LCMS, the reaction solution was diluted with ethyl acetate (50 mL), filtered, and the filter cake was washed with ethyl acetate. The filtrate was concentrated and purified by silica gel column chromatography (dichloromethane:methanol=20:1) to obtain a yellow solid Int-1 (800 mg, yield 75%). ESI-MS (m/z): 269.2 [M+H]⁺.

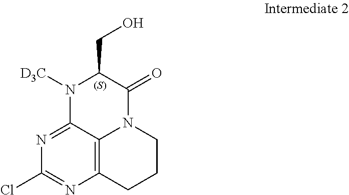

Intermediate 2 was prepared by the following steps:

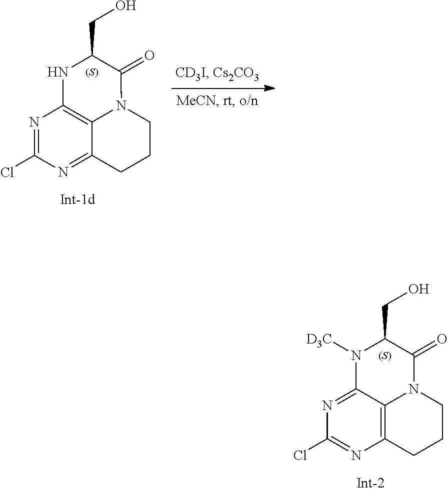

Step 1: Add compound Int-1d (2.0 g, 7.85 mmol) and cesium carbonate (5.12 g, 15.71 mmol) into a 100 mL single-necked flask, add acetonitrile (30 mL), then add deuterated iodomethane (3.42 g, 23.56 mmol) dropwise, and react at room temperature overnight. After the reaction was completed as monitored by LCMS, the reaction solution was diluted with ethyl acetate (50 mL), filtered, and the filter cake was washed with ethyl acetate. The filtrate was concentrated and purified by silica gel column chromatography (dichloromethane:methanol=20:1) to obtain a yellow solid Int-2 (1.3 g, yield 60%). ESI-MS (m/z): 272.2 [M+H]⁺.

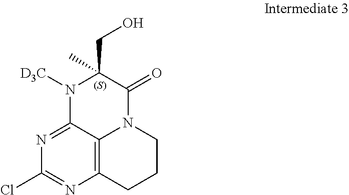

Intermediate 3 was prepared by the following steps:

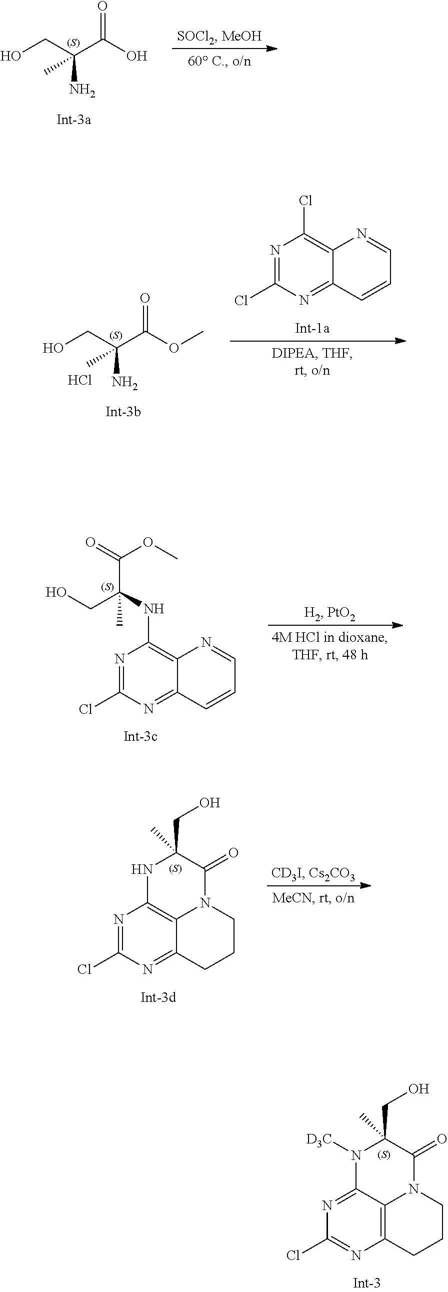

Step 1: Dissolve Int-3a (5.0 g, 41.97 mmol) in methanol (50 mL), place in an ice-water bath, and slowly add thionyl chloride (14.99 g, 125.92 mmol, 9.14 mL). After the addition was complete, warm to room temperature and then to 60° C. for reaction overnight. After the reaction was completed as monitored by LCMS, the reaction solution was concentrated to obtain a white solid Int-3b (6.5 g, yield 91%). ESI-MS (m/z): 170.2 [M+H]⁺.

Step 2: Dissolve compound Int-1a (7.0 g, 35.00 mmol), Int-3b (6.5 g, 38.50 mmol) and N, N-diisopropylethylamine (13.57 g, 104.99 mmol) in tetrahydrofuran (70 mL) and react at room temperature overnight. After the reaction was completed as monitored by LCMS, water (150 mL) was added, the mixture was filtered, the filter cake was washed with water, and then dried to obtain a white solid Int-3c (9.0 g, yield 86%). ESI-MS (m/z): 297.2 [M+H]⁺.

Step 3: Dissolve compound Int-3c (9.0 g, 30.33 mmol), platinum dioxide (688 mg, 3.03 mmol) and hydrochloric acid-1,4-dioxane solution (7.58 mL, 30.33 mmol, 4M) in tetrahydrofuran (100 mL), replace with hydrogen balloon three times and react at room temperature for 48 hours. After the reaction was completed as monitored by LCMS, methanol (150 mL) was added for dilution, the mixture was filtered, the filter cake was washed with methanol, the filtrate was alkalized with ammonia methanol (7M) (pH=9-10), then filtered, the filter cake was washed with water, and then dried to obtain a gray solid Int-3d (5.5 g, yield 67%). ESI-MS (m/z): 269.3 [M+H]⁺.

Step 3: Add compound Int-3d (2.0 g, 7.44 mmol) and cesium carbonate (4.85 g, 14.89 mmol) into a 100 mL single-necked flask, add acetonitrile (30 mL), and then add deuterated iodomethane (1.62 g, 11.16 mmol) dropwise, and react at room temperature overnight. After the reaction was completed as monitored by LCMS, the reaction solution was diluted with ethyl acetate (100 mL), filtered, and the filter cake was washed with ethyl acetate. The filtrate was concentrated and purified by silica gel column chromatography (dichloromethane:methanol=20:1) to obtain a yellow solid Int-3 (1.5 g, yield 70%). ESI-MS (m/z): 286.3 [M+H]⁺.

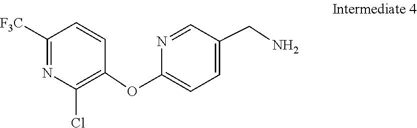 [SMILES: NCc1ccc(Oc2ccc(C(F)(F)F)nc2Cl)nc1]

Intermediate 4 was prepared by the following steps:

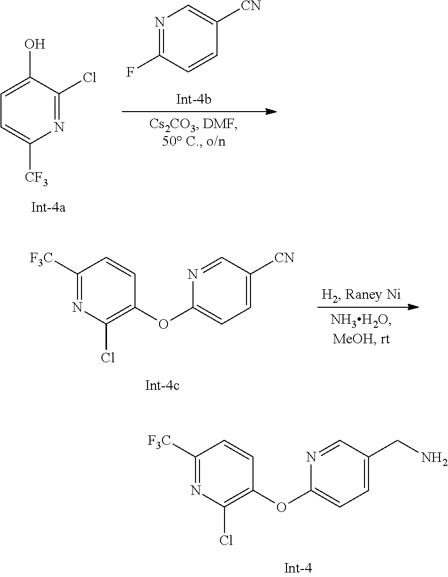

Step 1: Add compound Int-4a (700 mg, 3.54 mmol), Int-4b (433 mg, 3.54 mmol) and cesium carbonate (2.31 g, 7.09 mmol) to N, N-dimethylformamide (10 mL) and react at 50° C. for 16 hours. The reaction was completed as monitored by LCMS. N, N-dimethylformamide was distilled off under reduced pressure, and water and ethyl acetate were added for extraction. The organic phases were combined and washed with saturated brine, dried over anhydrous sodium sulfate, and the organic phase was filtered and concentrated to obtain a white solid Int-4c (711 mg, yield 67%).

Step 2: Compound Int-4c (355 mg, 1.18 mmol) was dissolved in methanol (35 mL).

Ammonia water (3.5 mL) and Raney nickel (3.5 mL, aqueous suspension) were added to the reaction system in sequence. The hydrogen was replaced by a hydrogen balloon and the mixture was reacted under a hydrogen atmosphere at room temperature for 3 hours. The reaction was completed as monitored by LCMS. The reaction solution was diluted with methanol and filtered. The filtrate was concentrated and purified by silica gel column chromatography (dichloromethane:methanol=9:1) to obtain brown oily liquid Int-4 (199 mg, yield 55%). ESI-MS (m/z): 304.2 [M+H]⁺.

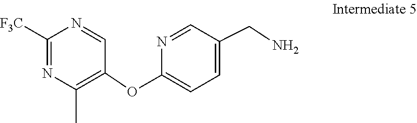 [SMILES: Cc1nc(C(F)(F)F)ncc1Oc1ccc(CN)cn1]

Intermediate 5 was prepared by the following steps:

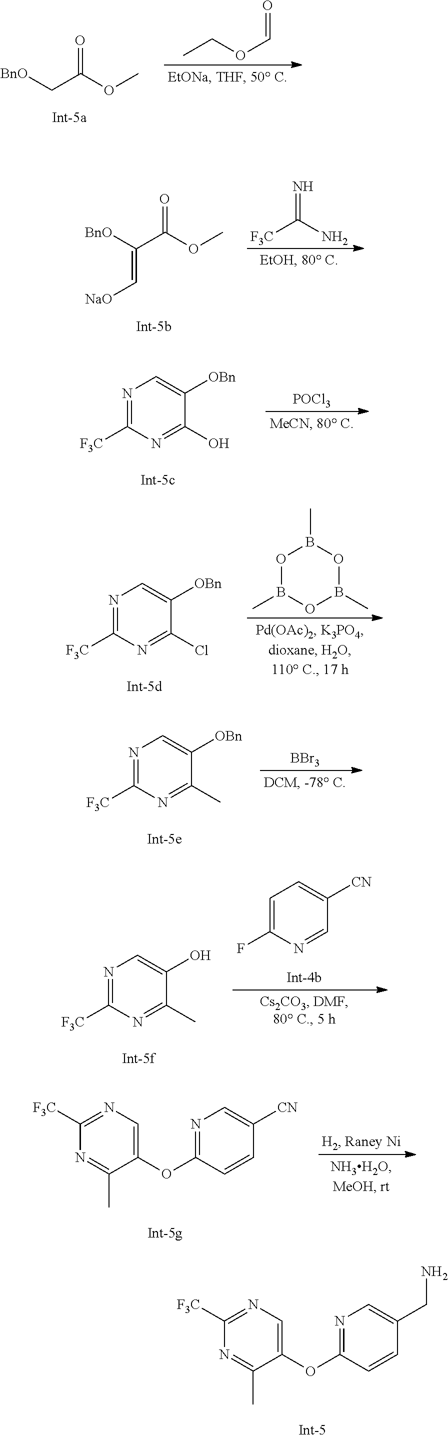

Step 1: Ethyl formate (3.17 g, 42.73 mmol) and solid sodium ethoxide (3.49 g, 50.50 mmol) were added to tetrahydrofuran (140 mL) in sequence. Compound Int-5a (7.0 g, 38.85 mmol) was added at 5-10° C., and the temperature was raised to 50° C. and stirred for 2 hours. The starting material disappeared as monitored by HPLC. THF was evaporated under reduced pressure to obtain a yellow oily substance Int-5b, which was used directly in the next step.

Step 2: Add 150 mL of anhydrous ethanol to the oily substance Int-5b obtained in the previous step, stir at room temperature to dissolve, add trifluoroacetamidine (4.35 g, 33.01 mmol, purity 85%) dropwise, keep stirring at 30° C. for 5 hours, then raise the temperature to 80° C. and continue stirring for 2 hours. HPLC monitoring shows that the raw material disappeared, cool down, evaporate about 100 mL of anhydrous ethanol, add the remaining residue to 300 mL of ice water, adjust the pH to 3 with concentrated hydrochloric acid, stir for 0.5 hour, filter with suction, and dry the filter cake to obtain a yellow solid compound Int-5c (4.37 g, two-step reaction yield 41%, purity 99%). ESI-MS (m/z): 271.4 [M+H]⁺.

Step 3: Add compound Int-5c (4.0 g, 14.80 mmol) to 60 mL of acetonitrile, add phosphorus oxychloride (6.81 g, 44.41 mmol) dropwise, stir for 10 minutes after the addition was complete, heat to 80° C., keep stirring for 2 hours, and monitor the complete conversion of the raw material by HPLC. The acetonitrile was removed under reduced pressure, and the residual liquid was added into 200 mL of ice water, stirred for 0.5 h, and filtered to obtain a yellow solid Int-5d (3.9 g, yield 86%, purity 95%).

Step 4: Compound Int-5d (2.0 g, 6.93 mmol), trimethylcyclotriboroxine (2.61 g, 20.79 mmol), palladium acetate (155 mg, 0.69 mol), and potassium phosphate (2.94 g, 13.86 mmol) were added to 1,4-dioxane (150 mL) in sequence, and water (15 mL) was added. Under nitrogen protection, the mixture was stirred at 90° C. for 17 hours. The conversion of the raw materials was complete as monitored by HPLC, and the filtrate was concentrated. The residue was purified by silica gel column chromatography (petroleum ether/ethyl acetate=1/9) to obtain Int-5e (1.22 g, yield 65%, purity 99%) as a white solid. ESI-MS (m/z): 269.1 [M+H]⁺.

Step 5: Add compound Int-5e (1.0 g, 3.73 mmol) to 20 ml methanol, add palladium carbon (10%, 100 mg), replace hydrogen in the reaction system and stir overnight at room temperature, filter the reaction solution through diatomaceous earth, and concentrate the filtrate to obtain compound Int-5f (630 mg, yield 92%, purity 97%). ESI-MS (m/z): 177.1 [M−H]⁻.

Step 6: Compound Int-5f (0.5 g, 2.81 mmol), Cs₂CO₃ (1.83 g, 5.61 mmol), and Int-4b (514 mg, 4.21 mmol) were added to N, N-dimethylformamide (10 mL) in sequence, and stirred at 20° C. overnight. The starting material disappeared as monitored by HPLC. The reaction solution was added to 50 ml of ice water, stirred for 0.5 h, and filtered to obtain a yellow solid Int-5g (510 mg, yield 64%, purity 99%). ESI-MS (m/z): 281.3 [M+H]⁺.

Step 7: Dissolve compound Int-5g (100 mg, 0.36 mmol) in methanol (5 mL), add ammonia water (0.2 mL), add Raney Nickel (0.5 mL, aqueous suspension), replace hydrogen in the reaction system and stir at room temperature for 2 hours. The reaction solution was filtered through celite, and the filtrate was concentrated. Compound Int-5 (95 mg, yield 93%, purity 90%) was obtained, ESI-MS (m/z): 284.9 [M+H]⁺.

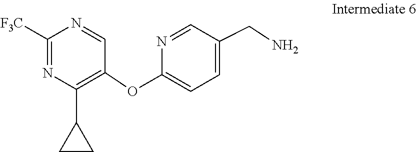 [SMILES: NCc1ccc(Oc2cnc(C(F)(F)F)nc2C2CC2)nc1]

Intermediate 6 was prepared by the following steps:

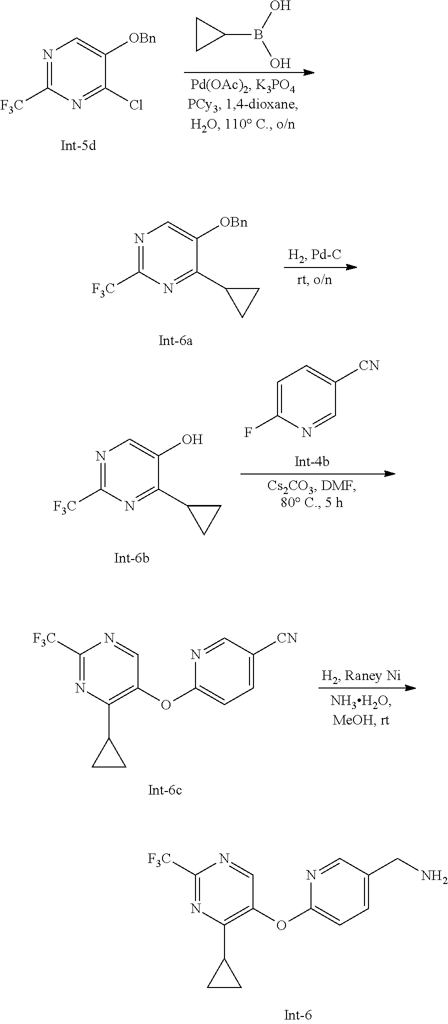

Step 1: Add compound Int-5d (550 mg, 1.91 mmol), cyclopropylboronic acid (818 mg, 9.53 mmol), palladium acetate (42 mg, 0.19 mmol), tricyclohexylphosphine (106 mg, 0.38 mmol) and potassium phosphate (1.21 g, 5.72 mmol) to a mixed solution of 1,4-dioxane (50 mL) and water (5 mL). After replacing nitrogen in the reaction system, react at 110° C. under nitrogen atmosphere for 16 hours. The reaction was completed as monitored by LCMS. The solvent was distilled off under reduced pressure, and water and ethyl acetate were added for extraction. The organic phases were combined and washed with saturated brine and dried over anhydrous sodium sulfate. The organic phase was concentrated and purified by silica gel column chromatography (petroleum ether:ethyl acetate=5:1) to obtain a white solid Int-6a (530 mg, yield 94%). ESI-MS (m/z): 295.3 [M+H]⁺.

Step 2: Compound Int-6a (530 mg, 1.80 mmol) was dissolved in methanol (10 mL), palladium carbon (10%, 21 mg) was added to the reaction system, hydrogen was replaced by a hydrogen balloon and the reaction was carried out under a hydrogen atmosphere at room temperature for 16 hours. The reaction was completed as monitored by LCMS. The reaction solution was diluted with methanol and filtered, and the filtrate was concentrated to obtain brown oily liquid Int-6b (330 mg, yield 89%). ESI-MS (m/z): 203.3 [M−H]⁻.

Step 3: Add compound Int-6b (330 mg, 1.62 mmol), Int-4b (236 mg, 1.94 mmol) and cesium carbonate (1.05 g, 3.23 mmol) to acetonitrile (10 mL) and react at room temperature for 16 hours. The reaction was completed as monitored by LCMS. The acetonitrile was distilled off under reduced pressure, and water and ethyl acetate were added for extraction. The organic phases were combined and washed with saturated brine and dried over anhydrous sodium sulfate. The organic phase was concentrated and purified by silica gel column chromatography (petroleum ether:ethyl acetate=3:1) to obtain a white solid Int-6c (450 mg, yield 90%). ESI-MS (m/z): 307.1 [M+H]⁺.

Step 4: Dissolve compound Int-6c (350 mg, 1.14 mmol) in methanol (20 mL), add aqueous ammonia (3.5 mL) and Raney nickel (3.5 mL, aqueous suspension) to the reaction system in sequence, replace hydrogen with a hydrogen balloon and react in a hydrogen atmosphere at room temperature for 16 hours. The reaction was completed as monitored by LCMS. The reaction solution was diluted with methanol and filtered through diatomaceous earth. The filtrate was concentrated and purified by silica gel column chromatography (dichloromethane:methanol=9:1) to obtain yellow oily liquid Int-6 (300 mg, yield 84%). ESI-MS (m/z): 311.2 [M+H]⁺.

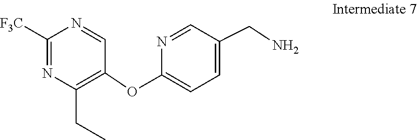 [SMILES: CCc1nc(C(F)(F)F)ncc1Oc1ccc(CN)cn1]

Intermediate 7 was prepared by the following steps:

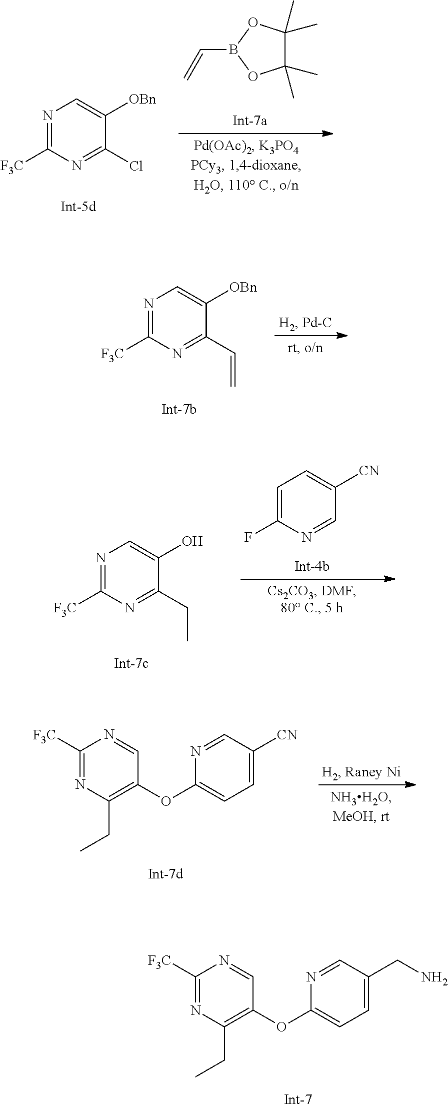

Step 1: Add compound Int-5d (2.0 g, 6.93 mmol), Int-7a (3.20 g, 20.79 mmol), palladium acetate (155 mg, 0.69 mol), and potassium phosphate (2.94 g, 13.86 mmol) to 1,4-dioxane (150 mL) in sequence, add water (15 mL), and stir at 90° C. under nitrogen protection for 17 hours. HPLC monitored that the raw material conversion was complete, and the filtrate was concentrated. The residue was purified by silica gel column chromatography (petroleum ether/ethyl acetate=1/9) to obtain Int-7b (1.18 g, yield 60%, purity 61%) as a white solid. ESI-MS (m/z): 281.1 [M+H]⁺.

Step 2: Add compound Int-7b (1.0 g, 3.73 mmol) to 20 ml methanol, add palladium carbon (10%, 100 mg), replace hydrogen in the reaction system and stir overnight at room temperature, filter the reaction solution with diatomaceous earth, and concentrate the filtrate to obtain compound Int-7c (677 mg, yield 94%, purity 97%). ESI-MS (m/z): 191.3 [M−H]⁻.

Step 3: Compound Int-7c (0.5 g, 2.81 mmol), Cs₂CO₃ (1.83 g, 5.61 mmol), and Int-4b (514 mg, 4.21 mmol) were added to N, N-dimethylformamide (10 mL) in sequence, stirred at 20° C. overnight, and the disappearance of the starting material was monitored by HPLC. The reaction solution was added to 50 ml of ice water, stirred for 0.5 h, and filtered to obtain a yellow solid Int-7d (530 mg, yield 64%, purity 99%). ESI-MS (m/z): 294.3 [M+H]⁺.

Step 4: Dissolve compound Int-7d (100 mg, 0.36 mmol) in methanol (5 mL), add ammonia water (0.2 mL), add Raney nickel (0.5 mL, aqueous suspension), replace hydrogen in the reaction system and stir at room temperature for 2 hours. The reaction solution was filtered through celite and the filtrate was concentrated to obtain compound Int-7 (97 mg, yield 93%, purity 90%), ESI-MS (m/z): 298.5 [M+H]⁺.

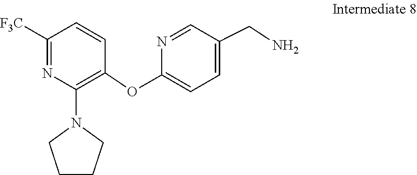 [SMILES: NCc1ccc(Oc2ccc(C(F)(F)F)nc2N2CCCC2)nc1]

Intermediate 8 was prepared by the following steps:

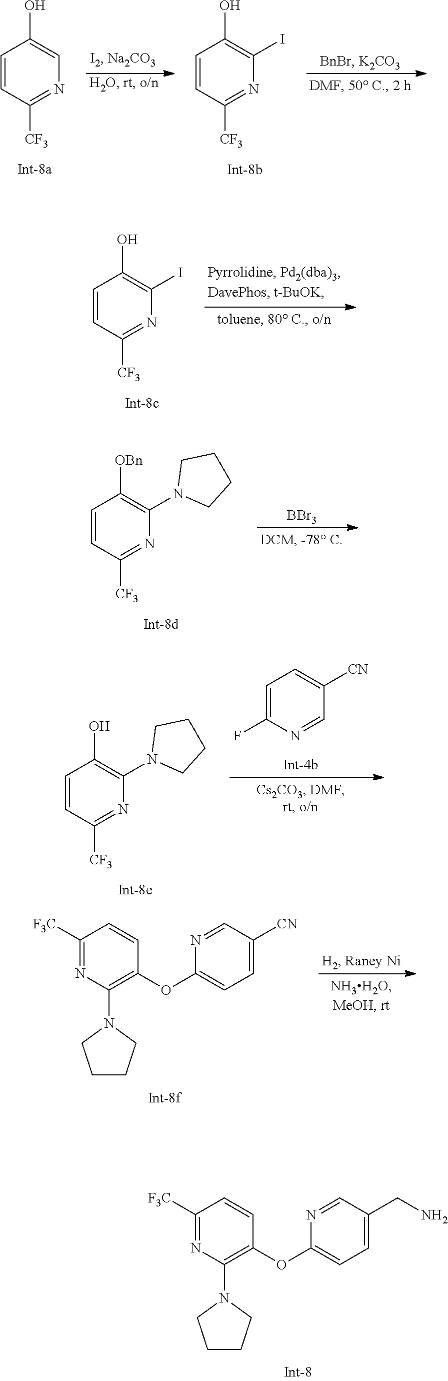

Step 1: Dissolve compound Int-8a (4.06 g, 24.89 mmol) and sodium carbonate (5.28 g, 49.79 mmol) in water (80 mL), then add iodine (6.32 g, 24.89 mmol), stir at room temperature for 16 hours, and add saturated sodium thiosulfate (40 mL) and ethyl acetate (40 mL) to the reaction solution. The pH value of the reaction solution was adjusted to 6 by adding concentrated hydrochloric acid, and then extracted with ethyl acetate. The organic phase was washed with saturated aqueous sodium chloride solution, dried over anhydrous sodium sulfate, filtered and concentrated, and the residue was purified by silica gel column chromatography (petroleum ether/ethyl acetate=4/1) to obtain a white solid Int-8b (3.1 g, yield 43%). ESI-MS (m/z): 290.2 [M+H]⁺.

Step 2: Compound Int-8b (2.33 g, 6.85 mmol) and potassium carbonate (1.76 g, 10.28 mmol) were dissolved in N, N-dimethylformamide (15 mL), followed by the addition of benzyl bromide (1.42 g, 10.28 mmol). The mixture was reacted at 50° C. for 2 h, and the reaction was completed as monitored by LCMS. Water (20 mL) was then added, followed by extraction with ethyl acetate. The organic phase was washed with saturated aqueous sodium chloride solution, dried over anhydrous sodium sulfate, filtered and concentrated. The residue was purified by silica gel column chromatography (petroleum ether/ethyl acetate=10/1) to give a white solid Int-8c (2.1 g, yield 69%) ESI-MS (m/z): 380.2 [M+H]⁺.

Step 3: Dissolve compound Int-8c (450 mg, 1.18 mmol), tetrahydropyrrole (126 mg, 1.78 mmol), tri(dibenzylideneacetone)dipalladium (108 mg, 0.12 mmol), potassium tert-butoxide (199 mg, 1.78 mmol), and 2-dicyclohexylphosphino-2′-(N, N-dimethylamino)-biphenyl (139 mg, 0.36 mmol) in toluene. After replacing nitrogen in the reaction system, react at 80° C. under nitrogen atmosphere for 16 hours. The reaction was completed as monitored by LCMS. The reaction solution was filtered through celite to remove insoluble matter and washed with ethyl acetate. The filtrate was concentrated and the residue was purified by silica gel column chromatography (petroleum ether/ethyl acetate=10/1) to obtain yellow oil Int-8d (206 mg, yield 54%). ESI-MS (m/z): 323.3 [M+H]⁺.

Step 4: Dissolve compound Int-8d (206 mg, 0.64 mmol) in dichloromethane (5 mL), lower the temperature of the reaction solution to −78° C., then add boron tribromide (801 mg, 3.20 mmol), react at −78° C. for 2 hours, and monitor the completion of the reaction by LCMS. Water (5 mL) was added, and the reaction mixture was heated to room temperature. The pH value of the reaction mixture was adjusted to 6 with 4M sodium hydroxide solution, and extracted with dichloromethane. The organic phase was washed with saturated aqueous sodium chloride solution, dried over anhydrous sodium sulfate, filtered and concentrated, and the residue was purified by silica gel column chromatography (petroleum ether/ethyl acetate=1/1) to obtain yellow oily liquid Int-8e (120 mg, yield 81%). ESI-MS (m/z): 233.1 [M+H]⁺.

Step 5: Dissolve compound Int-8e (100 mg, 0.43 mmol), compound In-4b (63 mg, 0.51 mmol) and cesium carbonate (280 mg, 0.86 mmol) in N, N-dimethylformamide (5 mL), stir at room temperature for 16 hours, and monitor the completion of the reaction by LCMS. Water (50 mL) was added and filtered under reduced pressure to obtain a light yellow solid Int-8f (103 mg, yield 72%). ESI-MS (m/z): 335.6 [M+H]⁺.

Step 6: Dissolve compound Int-8f (64 mg, 0.19 mmol) in methanol (10 mL), add ammonia water (1 mL) and Raney nickel (3 mL, aqueous suspension) to the reaction system in sequence, replace hydrogen with a hydrogen balloon and react in a hydrogen atmosphere at room temperature for 16 hours. The reaction was completed as monitored by LCMS. The reaction solution was filtered through celite, and the filtrate was concentrated and purified by silica gel column chromatography (dichloromethane:methanol=10/1) to obtain brown oily liquid Int-8 (53 mg, yield 84%). ESI-MS (m/z): 339.2 [M+H]⁺.

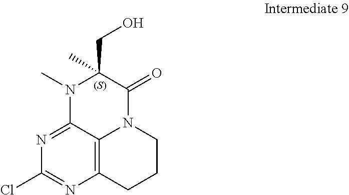

Intermediate 9 was prepared by the following steps:

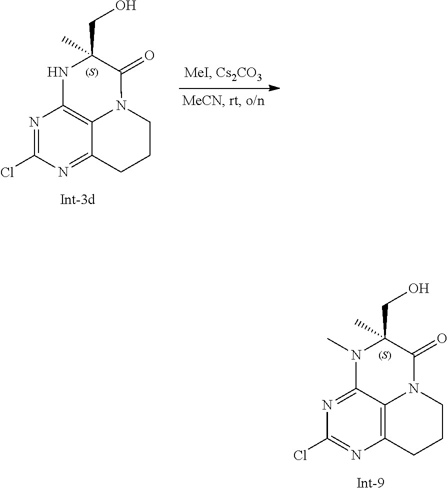

Step 1: Add compound Int-3d (2.0 g, 7.44 mmol) and cesium carbonate (4.85 g, 14.89 mmol) into a 100 mL single-necked flask, add acetonitrile (30 mL), then add iodomethane (2.11 g, 14.89 mmol) dropwise, and react at room temperature overnight. After the reaction was completed as monitored by LCMS, the reaction solution was diluted with ethyl acetate (100 mL), filtered, and the filter cake was washed with ethyl acetate. The filtrate was concentrated and purified by silica gel column chromatography (dichloromethane:methanol=20:1) to obtain a yellow solid Int-9 (1.7 g, yield 80%). ESI-MS (m/z): 283.3 [M+H]⁺.

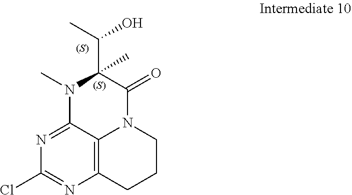

Intermediate 10 was prepared by the following steps:

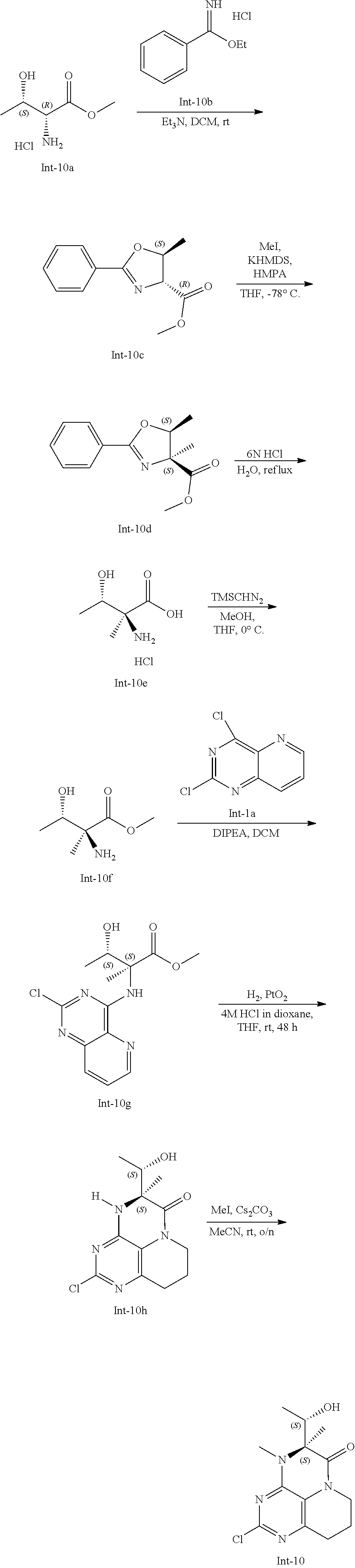

Step 1: To a solution of Int-10a (1.0 g, 5.9 mmol) and Int-10b (1.1 g, 5.9 mmol) in dichloromethane (15 mL) was added triethylamine (2.1 mL, 14.7 mmol) dropwise at room temperature. The reaction solution was stirred at room temperature for 24 hours, and the reaction was completed as monitored by LCMS. The reaction solution was diluted with dichloromethane, washed with saturated sodium bicarbonate aqueous solution and brine in sequence, and dried over anhydrous sodium sulfate. The organic phase was concentrated and purified by silica gel column chromatography (petroleum ether:ethyl acetate=4:1) to obtain colorless oily liquid Int-10c (642 mg, yield 50%). ESI-MS (m/z): 220.3 [M+H]⁺.

Step 2: Under nitrogen atmosphere and −78° C., potassium bis(trimethylsilyl)amide (2.8 mL, 12.2 mmol) and hexamethylphosphoric triamide (9.6 mL, 54.9 mmol) were added dropwise to a solution of compound Int-10c (2.23 g, 10.2 mmol) in tetrahydrofuran (20 mL). The reaction solution was stirred at −78° C. for 30 minutes, and then iodomethane (1.3 mL, 20.4 mmol) was added dropwise, and stirring was continued for 4 hours. The reaction was completed as monitored by LCMS. Saturated aqueous ammonium chloride solution was added to quench the reaction. The product was extracted with ethyl acetate. The organic phases were combined and washed with saturated brine and dried over anhydrous sodium sulfate. The organic phase was concentrated and purified by silica gel column chromatography (petroleum ether: ethyl acetate=4:1) to obtain a colorless oily liquid Int-10d (1.7 g, yield 72%). ESI-MS (m/z): 234.3 [M+H]⁺.

Step 3: Compound Int-10d (2.11 g, 9.0 mmol) was added to aqueous hydrochloric acid solution (35 mL, 6M), and the reaction was refluxed for 5 hours. The reaction was completed as monitored by LCMS. The reaction solution was concentrated by distillation under reduced pressure to obtain a crude product Int-10e.

Step 4: The crude product of Int-10e was dissolved in a mixed solvent of methanol (16 mL) and tetrahydrofuran (48 mL). Trimethylsilyldiazomethane (22.6 mL, 45.2 mmol, 2M in hexanes) was added dropwise under nitrogen atmosphere at 0° C. After reacting at room temperature for 16 hours, the reaction solution was concentrated by distillation under reduced pressure to obtain a crude product Int-10f. ESI-MS (m/z): 147.9 [M+H]⁺.

Step 5: The crude product of Int-10f, compound Int-1a (1.09 g, 5.45 mmol) and N, N-diisopropylethylamine (4.75 mL, 27.27 mmol) were dissolved in tetrahydrofuran (15 mL) and reacted at room temperature overnight. After the reaction was completed, tetrahydrofuran was distilled off under reduced pressure, extracted with ethyl acetate, and the organic phases were combined and washed with saturated brine and dried over anhydrous sodium sulfate. The organic phase was concentrated and purified by silica gel column chromatography (petroleum ether:ethyl acetate=3:2) to obtain a slightly yellow solid Int-10g (433 mg, yield 26%). ESI-MS (m/z): 311.1 [M+H]⁺.

Step 6: Add compound Int-10g (433 mg, 1.39 mmol), platinum dioxide (32 mg, 0.14 mmol) and a 1,4-dioxane solution of hydrochloric acid (0.70 mL, 2.79 mmol, 4M) to tetrahydrofuran (10 mL), replace with a hydrogen balloon three times and react at room temperature for 48 hours. After the reaction was completed as monitored by LCMS, methanol was added to dilute the reaction solution, and the reaction mixture was filtered. The filter cake was washed with methanol, and the filtrate was alkalized with aqueous sodium hydroxide solution (1 M) (pH=9-10), extracted with ethyl acetate, and the organic phases were combined and washed with saturated brine and dried over anhydrous sodium sulfate. After the organic phase was concentrated, it was purified by silica gel column chromatography (dichloromethane:methanol=20:1) to obtain a slightly yellow solid Int-10h (338 mg, yield 86%). ESI-MS (m/z): 283.3 [M+H]⁺.

Step 7: Compound Int-10h (159 mg, 0.56 mmol) and cesium carbonate (366 mg, 1.12 mmol) were added to acetonitrile (5 mL), and then iodomethane (120 mg, 0.84 mmol) was added dropwise thereto and reacted at room temperature overnight. After the reaction was completed as monitored by LCMS, acetonitrile was distilled off under reduced pressure, and the mixture was extracted with ethyl acetate. The organic phases were combined and washed with saturated brine, dried over anhydrous sodium sulfate, and the organic phase was concentrated to obtain a yellow solid Int-10. ESI-MS (m/z): 297.3 [M+H]⁺.

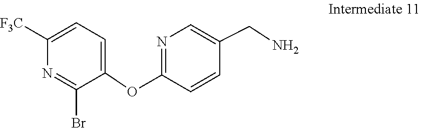 [SMILES: NCc1ccc(Oc2ccc(C(F)(F)F)nc2Br)nc1]

Intermediate 11 is prepared by the following steps:

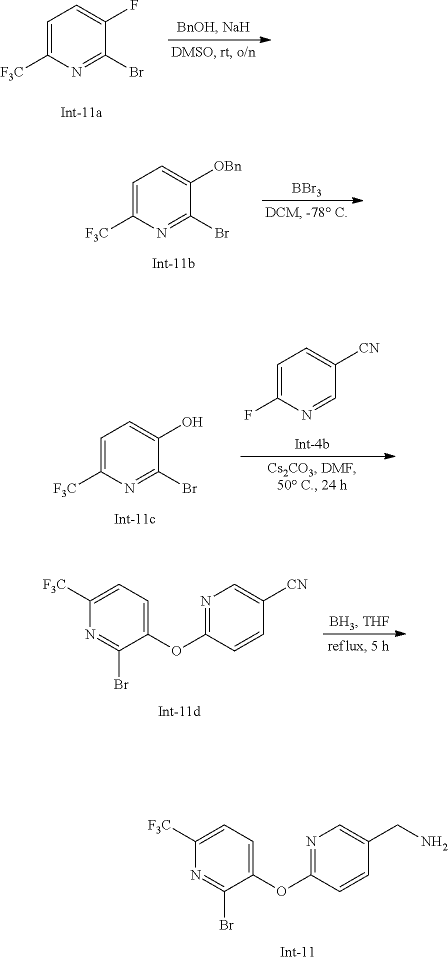

Step 1: Add a solution of benzyl alcohol (222 mg, 2.05 mmol) in dimethyl sulfoxide (1 mL) dropwise to a suspension of compound Int-11a (500 mg, 2.05 mmol) and sodium hydride (123 mg, 3.07 mmol, 60% in oil) in dimethyl sulfoxide (10 mL) and react at room temperature overnight. After the reaction was completed as monitored by LCMS, water was added to quench the reaction, and the product was extracted with ethyl acetate. The organic phases were combined and washed with saturated brine and dried over anhydrous sodium sulfate. The organic phase was concentrated and purified by silica gel column chromatography (petroleum ether:ethyl acetate=9:1) to obtain a white solid Int-11b (487 mg, yield 72%). ESI-MS (m/z): 332.1 [M+H]⁺.

Step 2: Add boron tribromide (0.42 mL, 4.40 mmol) dropwise to a solution of compound Int-11b (487 mg, 1.47 mmol) in dichloromethane (15 mL) at −78° C. and react overnight. After the reaction was completed as monitored by LCMS, methanol was added to quench the reaction, and the mixture was extracted with dichloromethane. The organic phases were combined and washed with saturated brine, dried over anhydrous sodium sulfate, and the organic phase was concentrated to obtain a white solid Int-11c. ESI-MS (m/z): 242.3[M+H]⁺.

Step 3: Add compound Int-tic (355 mg, 1.47 mmol), Int-4b (197 mg, 1.61 mmol) and cesium carbonate (956 mg, 2.93 mmol) to N, N-dimethylformamide (10 mL) and react at 50° C. for 24 hours. The raw material Int-tic still remained. Water was added to quench the reaction, and the product was extracted with ethyl acetate. The organic phases were combined and washed with saturated brine and dried over anhydrous sodium sulfate. The organic phase was concentrated and purified by silica gel column chromatography (petroleum ether:ethyl acetate=5:1) to obtain a white solid Int-11d (150 mg, yield 30%). ESI-MS (m/z): 345.1 [M+H]⁺.

Step 4: Under nitrogen atmosphere, borane (1.53 mL, 1.53 mmol, 1 N in THF) was added dropwise to a solution of compound Int-11d (150 mg, 0.44 mmol) in tetrahydrofuran (6 mL), and the reaction was refluxed for 5 h. After the reaction was completed as monitored by LCMS, methanol was added to quench the reaction. The reaction solution was concentrated and purified by silica gel column chromatography (dichloromethane:methanol=10:1) to obtain yellow transparent oily liquid Int-11 (74 mg, yield 49%). ESI-MS (m/z): 348.2 [M+H]⁺.

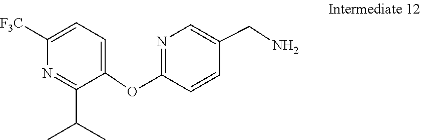 [SMILES: CC(C)c1nc(C(F)(F)F)ccc1Oc1ccc(CN)cn1]

Intermediate 12 is prepared by the following steps:

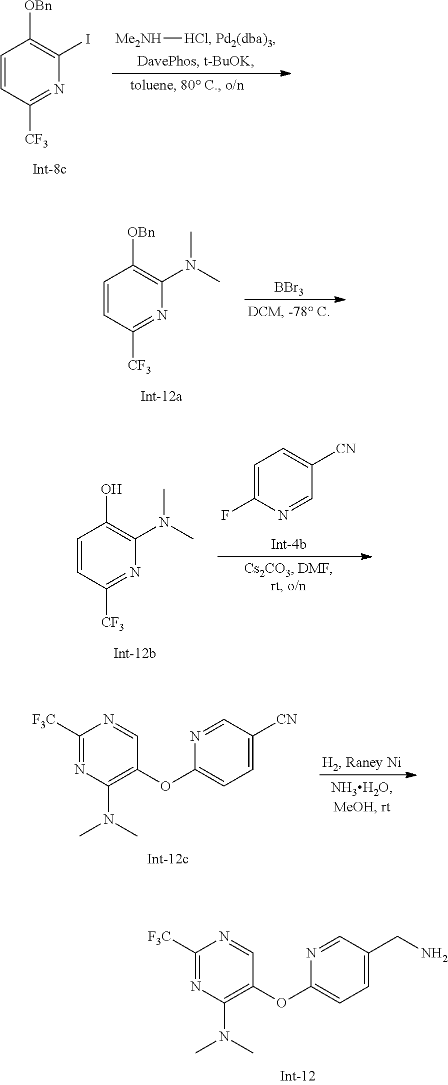

Step 1: Dissolve Int-8c (450 mg, 1.18 mmol), dimethylamine hydrochloride (145 mg, 1.78 mmol), tri(dibenzylideneacetone)dipalladium (108 mg, 0.12 mmol), potassium tert-butoxide (400 mg, 3.56 mmol), and 2-dicyclohexylphosphino-2′-(N,N-dimethylamino)-biphenyl (139 mg, 0.36 mmol) in toluene. After replacing nitrogen in the reaction system, react at 80° C. under nitrogen atmosphere for 16 hours. The reaction was completed as monitored by LCMS. The insoluble matter was filtered through celite, rinsed with ethyl acetate, and the filtrate was concentrated. The residue was purified by silica gel column chromatography (petroleum ether/ethyl acetate=10/1) to obtain Int-12a (224 mg, yield 54%) as a yellow oily liquid. MS (m/z): 297.2 [M+H]⁺.

Step 2: Dissolve compound Int-12a (151 mg, 0.51 mmol) in dichloromethane (10 mL), lower the temperature of the reaction solution to −78° C., then add boron tribromide (640 mg, 2.55 mmol), react at −78° C. for 2 hours, and monitor the completion of the reaction by LCMS. Water (10 mL) was added, and the reaction mixture was heated to room temperature. The pH value of the reaction mixture was adjusted to 6 with 4 M sodium hydroxide solution, and extracted with dichloromethane. The organic phase was washed with saturated aqueous sodium chloride solution, dried over anhydrous sodium sulfate, filtered and concentrated, and the residue was purified by silica gel column chromatography (petroleum ether/ethyl acetate=1/1) to obtain yellow oily liquid Int-12b (98 mg, yield 93%). MS (m/z): 207.4 [M+H]⁺.

Step 3: Dissolve Int-12b (98 mg, 0.48 mmol), Int-4b (63 mg, 0.51 mmol) and cesium carbonate (280 mg, 0.86 mmol) in N, N-dimethylformamide (5 mL) and stir at room temperature for 16 h. The reaction was completed as monitored by LCMS. Water (50 mL) was added and filtered under reduced pressure to obtain a light yellow solid Int-12c (102 mg, yield 70%). MS (m/z): 310.2 [M+H]⁺.

Step 4: Dissolve compound Int-12c (102 mg, 0.36 mmol) in methanol (10 mL), add ammonia water (1 mL) and Raney nickel (3 mL, aqueous suspension) to the reaction system in sequence, replace hydrogen with a hydrogen balloon and react under hydrogen atmosphere at room temperature for 16 hours. The reaction was completed as monitored by LCMS. The reaction solution was filtered through celite, and the filtrate was concentrated and purified by silica gel column chromatography (dichloromethane:methanol=10/1) to obtain brown oily liquid Int-12 (106 mg, yield 99%). MS (m/z): 314.3 [M+H]⁺.

The synthesis method of the embodiment compounds of the present disclosure is as follows:

### Example 1

### (S)-2-(((6-((2-chloro-6-(trifluoromethyl)pyridin-3-yl)oxy)pyridin-3-yl)methyl)amino)-5-(hydroxymethyl)-4-(methyl-d3)-4,5,9,10-tetrahydro-6H,8H-pyrido[3,2,1-de]pteridin-6-one

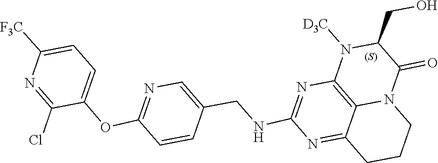

Example 1 was prepared by the following steps:

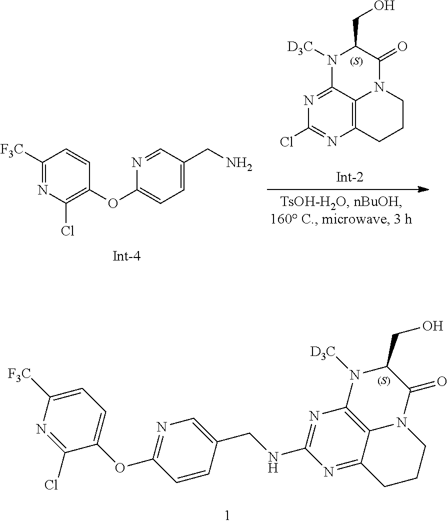

Step 1: Int-2 (42 mg, 0.15 mmol), Int-4 (47 mg, 0.15 mmol) and p-toluenesulfonic acid monohydrate (3 mg, 0.02 mmol) were dissolved in n-butanol (2 mL) and reacted at 160° C. in a microwave for 3 h. The reaction was complete as monitored by LCMS. The reaction solution was directly purified by reverse phase preparative HPLC to obtain a white solid 1 (41 mg, yield 50%). ESI-MS (m/z): 539.3[M+H]⁺; ¹H NMR (500 MHz, DMSO-d₆) δ 8.12-8.03 (m, 3H), 7.92 (dd, J=8.4, 2.4 Hz, 1H), 7.23 (d, J=8.4 Hz, 1H), 6.96 (s, 1H), 4.99 (t, J=5.6 Hz, 1H), 4.45-4.29 (m, 2H), 4.10-3.98 (m, 2H), 3.77-3.64 (m, 2H), 3.34-3.28 (m, 1H), 2.47 (t, J=4.3 Hz, 2H), 1.93-1.85 (m, 1H), 1.81-1.70 (m, 1H).

### Example 2

### (S)-2-(((6-((2-chloro-6-(trifluoromethyl)pyridin-3-yl)oxy)pyridin-3-yl)methyl)amino)-5-(hydroxymethyl)-4-methyl-4,5,9,10-tetrahydro-6H,8H-pyrido[3,2,1-de]pteridin-6-one

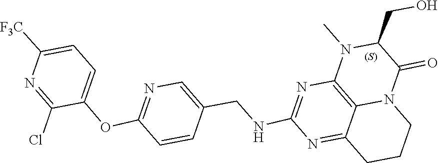

Example 2 was prepared by the following steps:

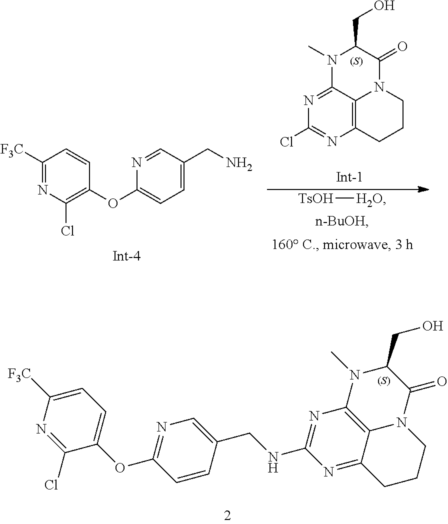

Step 1: Int-1 (41 mg, 0.15 mmol), Int-4 (47 mg, 0.15 mmol) and p-toluenesulfonic acid monohydrate (3 mg, 0.02 mmol) were dissolved in n-butanol (2 mL) and reacted at 160° C. in a microwave for 3 h. The reaction was complete as monitored by LCMS. The reaction solution was purified by reverse phase preparative HPLC to obtain a white solid 1 (45 mg, yield 55%). ESI-MS (m/z): 536.2[M+H]⁺; ¹H NMR (500 MHz, DMSO-d₆) δ 8.13-8.02 (m, 3H), 7.92 (dd, J=8.4, 2.4 Hz, 1H), 7.23 (d, J=8.4 Hz, 1H), 6.97 (t, 1H), 4.99 (t, J=5.6 Hz, 1H), 4.44-4.29 (m, 2H), 4.05 (t, J=2.6 Hz, 1H), 4.04-3.98 (m, 1H), 3.77-3.65 (m, 2H), 3.34-3.28 (m, 1H), 2.95 (s, 3H), 2.49-2.45 (m, 2H), 1.93-1.84 (m, 1H), 1.82-1.70 (m, 1H).

### Example 3

### (S)-2-(((6-((2-chloro-6-(trifluoromethyl)pyridin-3-yl)oxy)pyridin-3-yl)methyl)amino)-5-(hydroxymethyl)-5-methyl-4-(methyl-d3)-4,5,9,10-tetrahydro-6H,8H-pyrido[3,2,1-de]pteridin-6-one

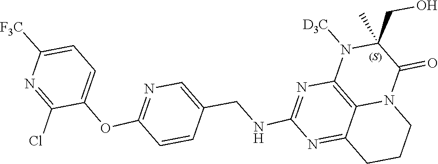

Example 3 was prepared by the following steps:

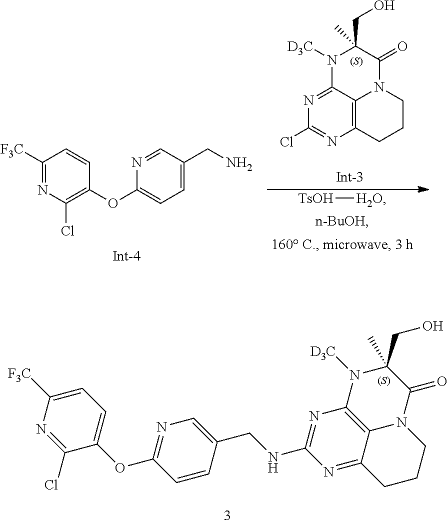

Step 1: Int-3 (44 mg, 0.15 mmol), Int-4 (47 mg, 0.15 mmol) and p-toluenesulfonic acid monohydrate (3 mg, 0.02 mmol) were dissolved in n-butanol (2 mL) and reacted at 160° C. in a microwave for 3 h. The reaction was complete as monitored by LCMS. The reaction solution was purified by reverse phase preparative HPLC to obtain a white solid 1 (34 mg, yield 40%). ESI-MS (m/z): 553.3[M+H]⁺; ¹H NMR (500 MHz, DMSO-d₆) δ 8.14-8.03 (m, 3H), 7.92 (dd, J=8.5, 2.4 Hz, 1H), 7.23 (d, J=8.4 Hz, 1H), 6.95 (s, 1H), 5.08 (t, J=5.5 Hz, 1H), 4.44-4.31 (m, 2H), 3.76-3.59 (m, 3H), 3.54 (dd, J=11.3, 5.4 Hz, 1H), 2.49-2.45 (m, 2H), 1.89-1.76 (m, 2H), 1.32 (s, 3H).

### Example 4

### (S)-5-(Hydroxymethyl)-4-(methyl-d3)-2-(((6-((4-methyl-2-(trifluoromethyl)pyrimidin-5-yl)oxy)pyridin-3-yl)methyl)amino)-4,5,9,10-tetrahydro-6H,8H-pyrido[3,2, 1-de]pteridin-6-one

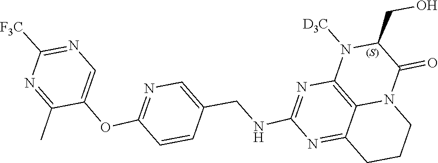

Example 4 was prepared by the following steps:

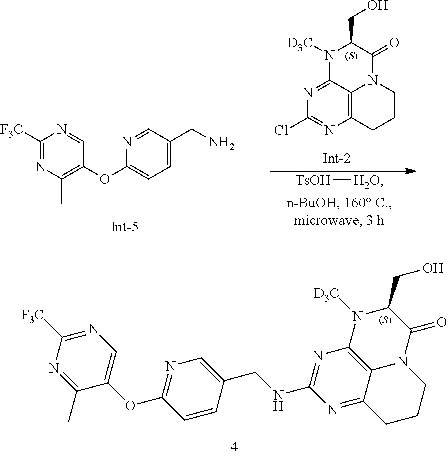

Step 1: Dissolve compound Int-5 (95 mg, 0.33 mmol) in n-butanol (3 mL), add compound Int-2 (89 mg, 0.33 mmol) and p-toluenesulfonic acid monohydrate (3 mg, 0.02 mmol), and stir the reaction solution at 160° C. for 3 hours under microwave conditions. The reaction solution was cooled to room temperature and concentrated under reduced pressure. The residue was purified by reverse phase preparative HPLC to obtain white solid 4 (31 mg, yield 18%, purity 99%). ESI-MS (m/z): 517.1 [M+H]⁺; ¹H NMR (500 MHz, DMSO-d₆) δ 8.87 (s, 1H), 8.08 (s, 1H), 7.97-7.88 (m, 1H), 7.24 (d, J=8.8, 3.2 Hz, 1H), 6.95 (s, 1H), 5.03-4.93 (m, 1H), 4.46-4.28 (m, 2H), 4.10-3.96 (m, 2H), 3.80-3.63 (m, 2H), 2.96 (s, 3H), 2.43 (s, 3H), 1.95-1.71 (m, 2H).

### Example 5

(S)-5-(Hydroxymethyl)-5-methyl-4-(methyl-d3)-2-(((6-((4-methyl-2-(trifluoromethyl) pyrimidin-5-yl)oxy)pyridin-3-yl)methyl)amino)-4,5,9,10-tetrahydro-6H,8H-pyrido[3,2,1-de]pteridin-6-one

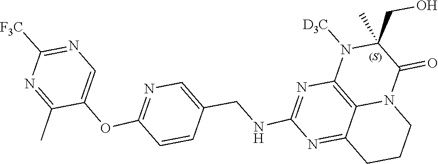

Example 5 was prepared by the following steps:

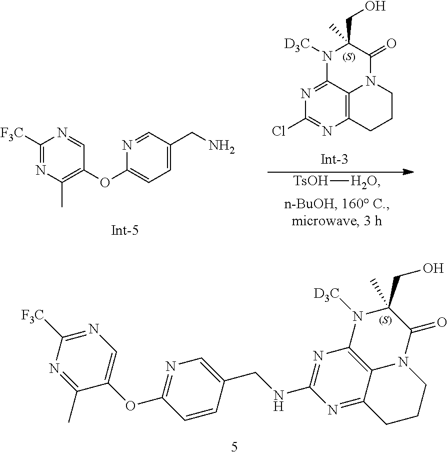

Step 1: Dissolve compound Int-5 (95 mg, 0.33 mmol) in n-butanol (3 mL), add compound Int-3 (94 mg, 0.33 mmol) and p-toluenesulfonic acid monohydrate (3 mg, 0.02 mmol), and stir the reaction solution at 160° C. for 3 hours under microwave conditions. The reaction solution was cooled to room temperature and concentrated under reduced pressure. The residue was purified by reverse phase preparative HPLC to obtain white solid 5 (28 mg, yield 15%, purity 99%). ESI-MS (m/z): 533.4 [M+H]⁺; ¹H NMR (500 MHz, DMSO-d₆) δ 8.87 (s, 1H), 8.08 (d, J=2.4 Hz, 1H), 7.92 (dd, J=8.5, 2.4 Hz, 1H), 7.23 (d, J=8.4 Hz, 1H), 6.93 (s, 1H), 5.06 (t, J=5.5 Hz, 1H), 4.45-4.30 (m, 3H), 3.80-3.66 (m, 2H), 3.66-3.58 (m, 1H), 3.59-3.49 (m, 1H), 2.50 (d, J=1.8 Hz, 2H), 2.43 (s, 3H), 1.89-1.77 (m, 2H), 1.33 (s, 3H).

### Example 6

### (S)-5-((S)-1-hydroxyethyl)-4,5-dimethyl-2-(((6-((4-methyl-2-(trifluoromethyl)pyrimidin-5-yl)oxy)pyridin-3-yl)methyl)amino)-4,5,9,10-tetrahydro-6H,8H-pyrido[3, 2,1-de]pteridin-6-one

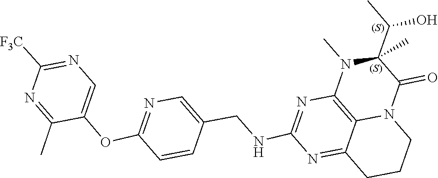

Example 6 was prepared by the following steps:

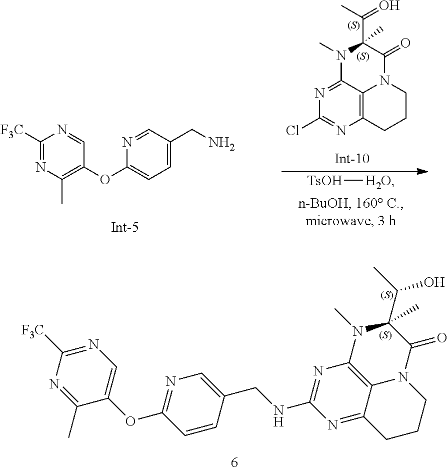

Step 1: Dissolve compound Int-5 (95 mg, 0.33 mmol) in n-butanol (3 mL), add compound Int-10 (97 mg, 0.33 mmol) and p-toluenesulfonic acid monohydrate (3 mg, 0.02 mmol), and stir the reaction solution at 160° C. for 3 hours under microwave conditions. The reaction solution was cooled to room temperature and concentrated under reduced pressure. The residue was purified by reverse phase preparative HPLC to obtain white solid 6 (37 mg, yield 20%, purity 99%). ESI-MS (m/z): 545.2 [M+H]⁺; ¹H NMR (500 MHz, DMSO-d₆) δ 8.86 (s, 1H), 8.08 (d, J=2.3 Hz, 1H), 7.92 (dd, J=8.5, 2.5 Hz, 1H), 7.23 (d, J=8.4 Hz, 1H), 7.00 (s, 1H), 4.85 (d, J=6.4 Hz, 1H), 4.46-4.39 (m, 1H), 4.37-4.30 (m, 1H), 4.06-3.99 (m, 1H), 3.81-3.71 (m, 1H), 3.31-3.22 (m, 2H), 3.05 (s, 3H), 2.43 (s, 3H), 1.91 (dd, J=6.8, 3.6 Hz, 1H), 1.77 (d, J=3.4 Hz, 1H), 0.95 (d, J=6.5 Hz, 3H).

### Example 7

(S)-2-(((6-((4-ethyl-2-(trifluoromethyl)pyrimidin-5-yl)oxy)pyridin-3-yl)methyl)amino)-5-(hydroxymethyl)-5-methyl-4-(methyl-d3)-4,5,9,10-tetrahydro-6H,8H-pyrido[3,2,1-de]pteridin-6-one

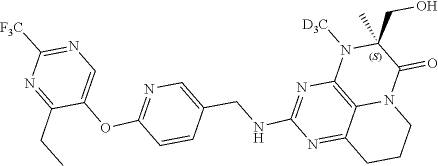

Example 7 was prepared by the following steps:

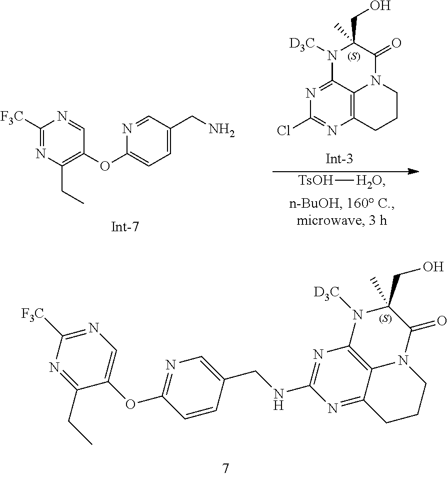

Step 1: Dissolve compound Int-7 (50 mg, 0.17 mmol) in n-butanol (3 mL), add compound Int-3 (47 mg, 0.17 mmol) and p-toluenesulfonic acid monohydrate (3 mg, 0.02 mmol), and stir the reaction solution at 160° C. for 3 hours under microwave conditions. The reaction solution was cooled to room temperature and concentrated under reduced pressure. The residue was purified by reverse phase preparative HPLC to obtain white solid 7 (12 mg, yield 13%, purity 99%). ESI-MS (m/z): 548.2 [M+H]⁺; ¹H NMR (500 MHz, DMSO-d₆) δ 8.87 (s, 1H), 8.08 (d, J=2.4 Hz, 1H), 7.92 (dd, J=8.4, 2.4 Hz, 1H), 7.24 (d, J=8.4 Hz, 1H), 6.93 (s, 1H), 5.06 (t, J=5.5 Hz, 1H), 4.41-4.30 (m, 2H), 3.78-3.66 (m, 2H), 3.65-3.60 (m, 1H), 3.58-3.50 (m, 1H), 2.81-2.71 (m, 2H), 2.49-2.43 (m, 2H), 1.90-1.76 (m, 2H), 1.32 (s, 3H), 1.17 (t, J=7.5 Hz, 3H).

### Example 8

### (S)-2-(((6-((4-ethyl-2-(trifluoromethyl)pyrimidin-5-yl)oxy)pyridin-3-yl)methyl)amino)-5-(hydroxymethyl)-4-(methyl-d3)-4,5,9,10-tetrahydro-6H,8H-pyrido[3,2,1-de]pteridin-6-one

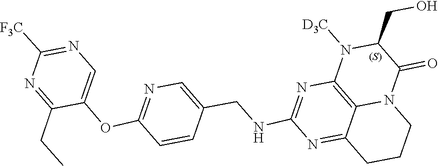

Example 8 was prepared by the following steps:

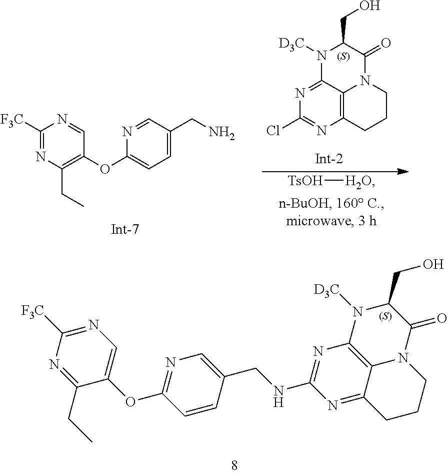

Step 1: Dissolve compound Int-7 (50 mg, 0.17 mmol) in n-butanol (3 mL), add compound Int-2 (45 mg, 0.17 umol) and p-toluenesulfonic acid monohydrate (3 mg, 0.02 mmol), and stir the reaction solution at 160° C. for 3 hours under microwave conditions. The reaction solution was cooled to room temperature and concentrated under reduced pressure. The residue was purified by reverse phase preparative HPLC to obtain white solid 8 (20 mg, yield 22%, purity 99%). ESI-MS (m/z): 533.4 [M+H]⁺; ¹H NMR (500 MHz, DMSO-d₆) δ 8.87 (s, 1H), 8.08 (d, J=2.4 Hz, 1H), 7.92 (dd, J=8.4, 2.5 Hz, 1H), 7.24 (d, J=8.4 Hz, 1H), 6.94 (s, 2H), 4.97 (t, J=5.5 Hz, 1H), 4.44-4.28 (m, 2H), 4.11-3.97 (m, 2H), 3.79-3.65 (m, 2H), 2.76 (q, J=7.5 Hz, 2H), 2.49-2.45 (m, 2H), 1.96-1.83 (m, 1H), 1.82-1.71 (m, 1H), 1.17 (t, J=7.5 Hz, 3H).

### Example 9

### (S)-2-(((6-((4-cyclopropyl-2-(trifluoromethyl)pyrimidin-5-yl)oxy)pyridin-3-yl)methyl) amino)-5-(hydroxymethyl)-4-(methyl-d3)-4,5,9,10-tetrahydro-6H,8H-pyrido[3,2,1-de]pteridin-6-one

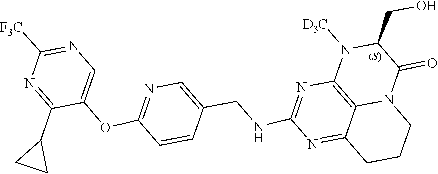

Example 9 was prepared by the following steps:

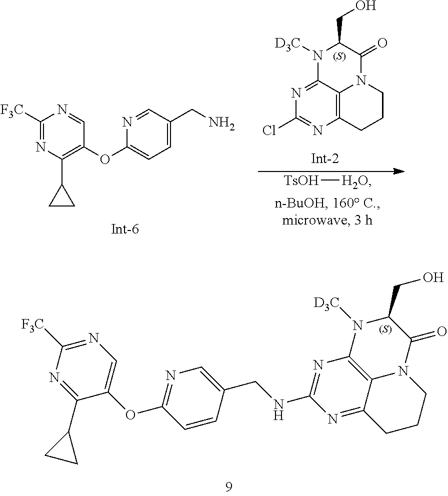

Step 1: Int-2 (50 mg, 0.18 mmol), Int-6 (57 mg, 0.18 mmol) and p-toluenesulfonic acid monohydrate (3 mg, 0.02 mmol) were dissolved in n-butanol (2 mL) and reacted at 160° C. in a microwave for 3 h. The reaction was complete as monitored by LCMS. The reaction solution was purified by reverse phase preparative HPLC to obtain a white solid 9 (65 mg, yield 60%). ESI-MS (m/z): 546.3[M+H]⁺; ¹H NMR (500 MHz, DMSO-d6) δ 8.78 (s, 1H), 8.09 (d, J=2.3 Hz, 1H), 7.92 (dd, J=8.5, 2.4 Hz, 1H), 7.25 (d, J=8.4 Hz, 1H), 6.94 (t, J=6.5 Hz, 1H), 4.97 (s, 1H), 4.46-4.30 (m, 2H), 4.10-3.99 (m, 2H), 3.71 (q, J=12.0, 11.4 Hz, 2H), 2.47 (t, J=6.6 Hz, 2H), 2.29-2.17 (m, 1H), 1.94-1.85 (m, 1H), 1.81-1.70 (m, 1H), 1.19-1.07 (m, 4H).

### Example 10

### (S)-2-(((6-((4-cyclopropyl-2-(trifluoromethyl)pyrimidin-5-yl)oxy)pyridin-3-yl)methyl) amino)-5-(hydroxymethyl)-5-methyl-4-(methyl-d3)-4,5,9,10-tetrahydro-6H,8H-pyrido[3,2,1-de]pteridin-6-one

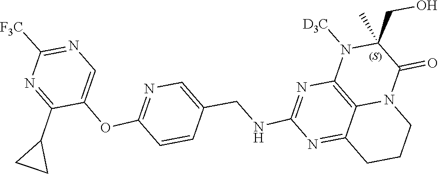

Example 10 was prepared by the following steps:

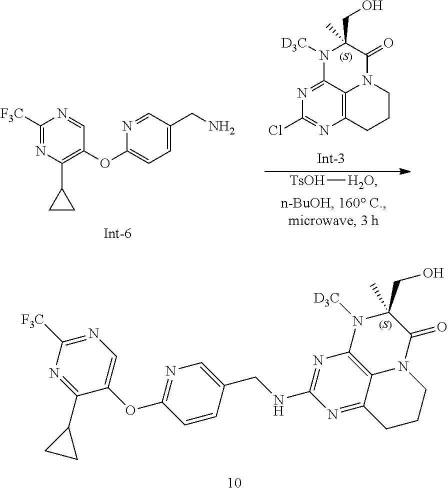

Step 1: Int-3 (50 mg, 0.17 mmol), Int-6 (54 mg, 0.17 mmol) and p-toluenesulfonic acid monohydrate (3 mg, 0.02 mmol) were dissolved in n-butanol (2 mL) and reacted at 160° C. in a microwave for 3 h. The reaction was complete as monitored by LCMS. The reaction solution was purified by reverse phase preparative HPLC to obtain a white solid 10 (43 mg, yield 44%). ESI-MS (m/z): 560.3[M+H]⁺; ¹H NMR (500 MHz, DMSO-d6) δ 8.79 (d, J=2.1 Hz, 1H), 8.09 (d, J=2.6 Hz, 1H), 7.92 (dt, J=8.4, 2.9 Hz, 1H), 7.26 (dd, J=8.4, 2.3 Hz, 1H), 6.95 (s, 1H), 5.15 (s, 1H), 4.50-4.28 (m, 2H), 3.76-3.52 (m, 4H), 2.50-2.44 (m, 2H), 2.30-2.18 (m, 1H), 1.91-1.77 (m, 2H), 1.33 (s, 1H), 1.18-1.07 (m, 4H).

### Example 11

### (S)-2-(((6-((4-cyclopropyl-2-(trifluoromethyl)pyrimidin-5-yl)oxy)pyridin-3-yl)methyl) amino)-5-((S)-1-hydroxyethyl)-5-methyl-4-(methyl-d3)-4,5,9,10-tetrahydro-6H,8H-pyrido[3,2,1-de]pteridin-6-one

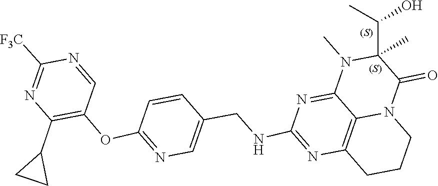

Example 11 was prepared by the following steps:

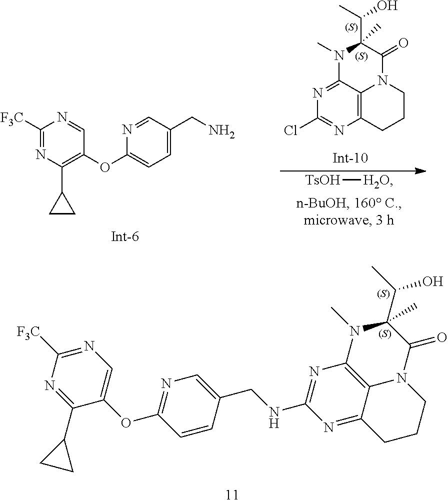

Step 1: Int-10 (50 mg, 0.17 mmol), Int-6 (52 mg, 0.17 mmol) and p-toluenesulfonic acid monohydrate (3 mg, 0.02 mmol) were dissolved in n-butanol (2 mL) and reacted at 160° C. in a microwave for 3 h. The reaction was complete as monitored by LCMS. The reaction solution was purified by reverse phase preparative HPLC to obtain a white solid 11 (11 mg, yield 12%). ESI-MS (m/z): 571.3[M+H]⁺; ¹H NMR (500 MHz, DMSO-d6) δ 8.78 (s, 1H), 8.09 (d, J=2.3 Hz, 1H), 7.93 (dd, J=8.5, 2.4 Hz, 1H), 7.27 (d, J=8.4 Hz, 1H), 7.13 (s, 1H), 4.93 (s, 1H), 4.48-4.32 (m, 2H), 4.10-3.99 (m, 1H), 3.77 (q, J=6.4 Hz, 1H), 3.29-3.22 (m, 1H), 3.06 (s, 3H), 2.53 (s, 2H), 2.26-2.19 (m, 1H), 1.98-1.90 (m, 1H), 1.82-1.73 (m, 1H), 1.53 (s, 3H), 1.15-1.08 (m, 4H), 0.96 (d, J=6.5 Hz, 3H).

### Example 12

### (S)-2-(((6-((2-bromo-6-(trifluoromethyl)pyridin-3-yl)oxy)pyridin-3-yl)methyl)amino)-5-(hydroxymethyl)-5-methyl-4-(methyl-d3)-4,5,9,10-tetrahydro-6H,8H-pyrid o[3,2,1-de]pteridin-6-one

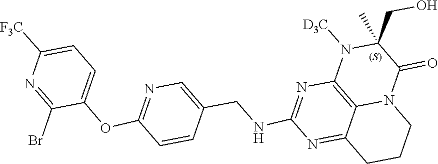

Example 12 was prepared by the following steps:

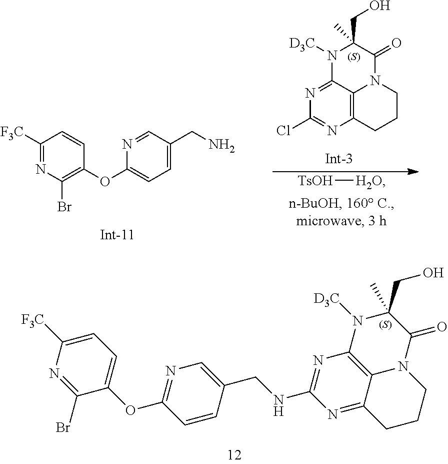

Step 1: Compound Int-3 (30 mg, 0.10 mmol), Int-11 (40 mg, 0.12 mmol) and p-toluenesulfonic acid monohydrate (2 mg, 0.01 mmol) were dissolved in n-butanol (2 mL) and reacted at 160° C. in a microwave for 3 h. The reaction was complete as monitored by LCMS. The reaction solution was purified by reverse phase preparative HPLC to obtain a white solid 12 (11 mg, yield 18%). ESI-MS (m/z): 553.3 [M+H]⁺; ¹H NMR (500 MHz, DMSO-d₆) δ 8.12-7.98 (m, 3H), 7.92 (dd, J=8.4, 2.4 Hz, 1H), 7.24 (d, J=8.4 Hz, 1H), 5.13 (t, J=5.3 Hz, 1H), 4.39 (t, J=6.5 Hz, 2H), 3.77-3.71 (m, 1H), 3.69 (dd, J=11.3, 5.7 Hz, 1H), 3.65-3.59 (m, 1H), 3.55 (dd, J=11.4, 5.3 Hz, 1H), 2.50-2.43 (m, 2H), 1.89-1.77 (m, 2H), 1.34 (s, 3H).

### Example 13

### (S)-5-(Hydroxymethyl)-4,5-dimethyl-2-(((6-((2-(pyrrolidin-1-yl)-6-(trifluoromethyl) pyridin-3-yl)oxy)pyridin-3-yl)methyl)amino)-4,5,9,10-tetrahydro-6H,8H-pyrido[3,2,1-de]pteridin-6-one

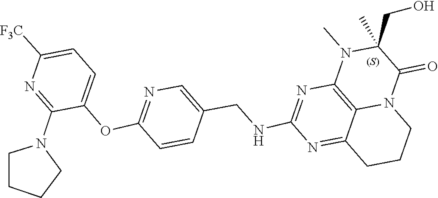

Example 13 was prepared by the following steps:

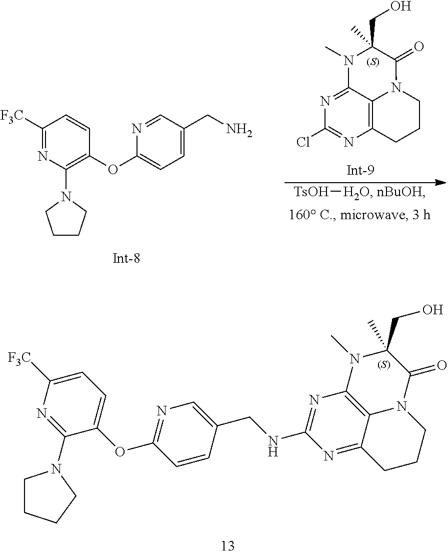

Step 1: Compound Int-8 (77 mg, 0.22 mmol), Int-9 (50 mg, 0.17 mmol) and p-toluenesulfonic acid monohydrate (3 mg, 17 umol) were dissolved in n-butanol (2 mL) and reacted at 160° C. in a microwave for 3 h. The reaction was complete as monitored by LCMS. The reaction solution was purified by reverse phase preparative HPLC to obtain a white solid 13 (44 mg, yield 43%). ESI-MS (m/z): 585.3 [M+H]⁺; ¹H NMR (500 MHz, DMSO-d6) δ 8.08 (d, J=2.3 Hz, 1H), 7.84 (dd, J=8.4, 2.4 Hz, 1H), 7.39 (d, J=7.8 Hz, 1H), 7.05 (d, J=8.0 Hz, 1H), 7.02 (d, J=8.1 Hz, 1H), 6.89 (br s, 1H), 5.06 (t, J=5.5 Hz, 1H), 4.43-4.28 (m, 2H), 3.81-3.67 (m, 2H), 3.67-3.59 (m, 1H), 3.57-3.53 (m, 1H), 3.50-3.40 (m, 4H), 2.94 (s, 3H), 2.55-2.45 (m, 2H), 1.87-1.70 (m, 6H), 1.33 (s, 3H).

### Example 14

### (S)-2-(((6-((4-ethyl-2-(trifluoromethyl)pyrimidin-5-yl)oxy)pyridin-3-yl)methyl)amino)-5-((S)-1-hydroxyethyl)-4,5-dimethyl-4,5,9,10-tetrahydro-6H,8H-pyrido[3,2,1-de]pteridin-6-one

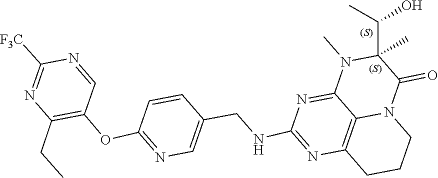

Example 14 was prepared by the following steps:

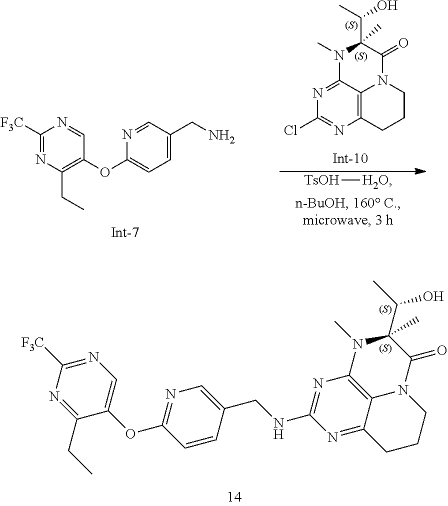

Step 1: Dissolve compound Int-7 (50 mg, 0.17 mmol) in n-butanol (3 mL), add compound Int-10 (50 mg, 0.17 mmol) and p-toluenesulfonic acid monohydrate (3 mg, 0.02 mmol), and stir the reaction solution at 160° C. for 3 hours under microwave conditions. The reaction solution was cooled to room temperature and concentrated under reduced pressure. The residue was purified by reverse phase preparative HPLC to obtain white solid 14 (22 mg, yield 23%, purity 99%). ESI-MS (m/z): 559.3[M+H]⁺; ¹H NMR (500 MHz, DMSO-d₆) δ 8.87 (s, 1H), 8.07 (s, 1H), 7.92 (d, J=8.1 Hz, 1H), 7.25 (d, J=8.5 Hz, 1H), 7.06 (s, 1H), 4.99-4.84 (m, 1H), 4.47-4.28 (m, 2H), 4.03 (d, J=13.0 Hz, 1H), 3.82-3.70 (m, 1H), 3.24 (t, J=11.8 Hz, 1H), 3.04 (s, 3H), 2.82-2.69 (m, 2H), 2.55-2.48 (m, 2H), 2.01-1.86 (m, 1H), 1.84-1.70 (m, 1H), 1.51 (s, 3H), 1.16 (t, J=7.6 Hz, 3H), 0.95 (d, J=6.4 Hz, 3H).

### Example 15

### (S)-2-(((6-((2-(dimethylamino)-6-(trifluoromethyl)pyridin-3-yl)oxy)pyridin-3-yl) methyl)amino)-5-(hydroxymethyl)-4,5-dimethyl-4,5,9,10-tetrahydro-6H,8H-pyrido[3,2,1-de]pteridin-6-one

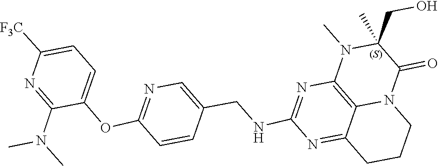

Example 15 was prepared by the following steps:

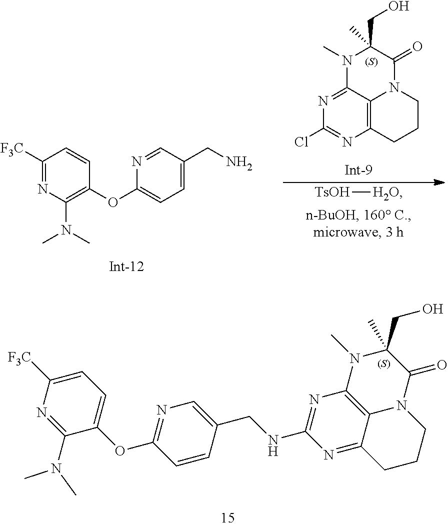

Step 1: Dissolve compound Int-12 (99 mg, 0.31 mmol) in n-butanol (2 mL), add compound Int-9 (50 mg, 0.17 mmol) and p-toluenesulfonic acid monohydrate (6 mg, 35 umol), and stir the reaction solution at 160° C. for 3 hours under microwave conditions. The reaction solution was cooled to room temperature and concentrated under reduced pressure. The residue was purified by reverse phase preparative HPLC to obtain white solid 15 (28 mg, yield 28%). ESI-MS (m/z): 559.3 [M+H]⁺; ¹H NMR (500 MHz, DMSO-d6) δ 8.07 (d, J=2.3 Hz, 1H), 7.84 (dd, J=8.5, 2.4 Hz, 1H), 7.46 (d, J=7.9 Hz, 1H), 7.17 (d, J=8.0 Hz, 1H), 7.07 (d, J=8.4 Hz, 1H), 6.89 (br s, 1H), 5.06 (t, J=5.4 Hz, 1H), 4.43-4.31 (m, 2H), 3.78-3.67 (m, 2H), 3.65-3.60 (m, 1H), 3.58-3.52 (m, 1H), 3.35-3.25 (m, 2H), 3.00-2.90 (m, 9H), 2.55-2.45 (m, 2H), 1.90-1.75 (m, 2H), 1.33 (s, 3H).

### Example 16

### (S)-5-(Hydroxymethyl)-4,5-dimethyl-2-(((6-((2-(methylamino)-6-(trifluoromethyl) pyridin-3-yl)oxy)pyridin-3-yl)methyl)amino)-4,5,9,10-tetrahydro-6H,8H-pyrido[3,2,1-de]pteridin-6-one

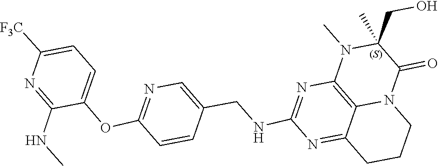

Example 16 was prepared by the following steps:

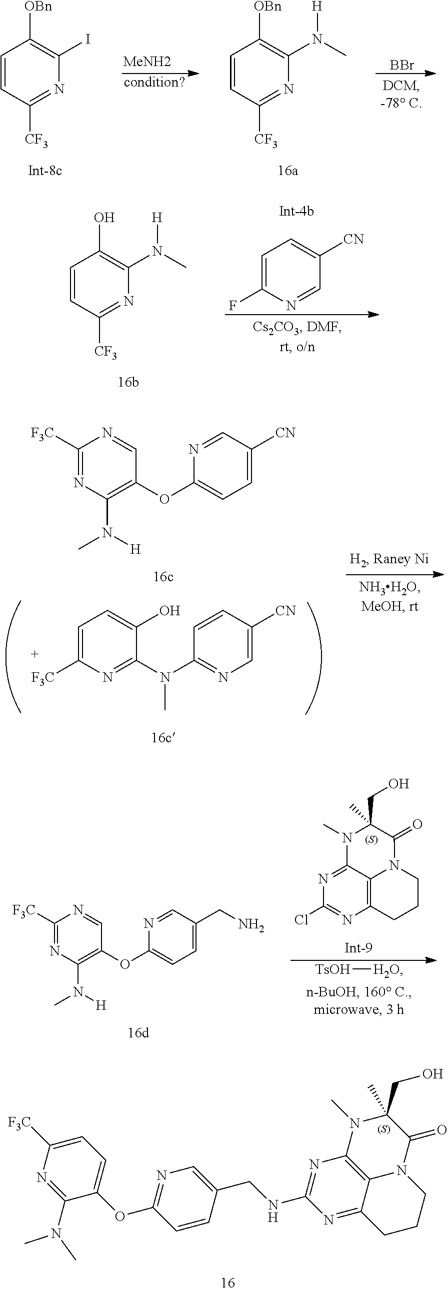

Step 1: Dissolve Int-8c (500 mg, 1.18 mmol), methylamine hydrochloride (133 mg, 1.98 mmol), tri(dibenzylideneacetone)dipalladium (120 mg, 0.13 mmol), potassium tert-butoxide (591 mg, 5.28 mmol), and 2-dicyclohexylphosphino-2′-(N,N-dimethylamino)-biphenyl (155 mg, 0.39 mmol) in toluene. After replacing nitrogen in the reaction system, react at 80° C. under nitrogen atmosphere for 16 hours. The reaction was completed as monitored by LCMS. The insoluble matter was filtered through celite, rinsed with ethyl acetate, and the filtrate was concentrated. The residue was purified by silica gel column chromatography (petroleum ether/ethyl acetate=10/1) to obtain yellow oily liquid 16a (278 mg, yield 73%). MS (m/z): 283.3 [M+H]⁺.

Step 2: Compound 16a (152 mg, 0.54 mmol) was dissolved in dichloromethane (3 mL), and the temperature of the reaction solution was lowered to −78° C. Then, boron tribromide (674 mg, 269 mmol) was added and the reaction was carried out at −78° C. for 2 hours. The reaction was completed as monitored by LCMS. Water (10 mL) was added, and the reaction mixture was heated to room temperature. The pH value of the reaction mixture was adjusted to 6 with 4 M sodium hydroxide solution, and extracted with dichloromethane. The organic phase was washed with saturated aqueous sodium chloride solution, dried over anhydrous sodium sulfate, filtered and concentrated. The residue was purified by silica gel column chromatography (petroleum ether/ethyl acetate=1/1) to obtain yellow oily liquid 16b (81 mg, yield 78%). MS (m/z): 193.4 [M+H]⁺.

Step 3: 16b (81 mg, 0.42 mmol), Int-4b (26 mg, 0.21 mmol) and cesium carbonate (137 mg, 0.42 mmol) were dissolved in N, N-dimethylformamide (2 mL) and stirred at room temperature for 16 h. The reaction was completed as monitored by LCMS. Water (10 mL) was added and the product was filtered off under reduced pressure to obtain a light yellow solid 16c (47 mg, yield 38%). MS (m/z): 295.2 [M+H]+.

Step 4: Compound 16c (47 mg, 0.16 mmol) was dissolved in methanol (10 mL). Ammonia water (1 mL) and Raney nickel (3 mL, aqueous suspension) were added to the reaction system in sequence. The hydrogen was replaced by a hydrogen balloon and the reaction was carried out under a hydrogen atmosphere at room temperature for 16 hours. The reaction was completed as monitored by LCMS. The reaction solution was filtered through celite, and the filtrate was concentrated and purified by silica gel column chromatography (dichloromethane:methanol=10/1) to obtain brown oily liquid 16d (45 mg, yield 94%). MS (m/z): 299.2 [M+H]+.

Step 5: Compound 16d (45 mg, 0.15 mmol) was dissolved in n-butanol (2 mL), and compound Int-9 (42 mg, 0.15 mmol) and p-toluenesulfonic acid monohydrate (3 mg, 0.02 mmol) were added. The reaction solution was stirred at 160° C. for 3 hours under microwave conditions. The reaction solution was cooled to room temperature and concentrated under reduced pressure. The residue was purified by reverse phase preparative HPLC to obtain white solid 16 (9 mg, yield 13%). ESI-MS (m/z): 545.4 [M+H]+; ¹H NMR (500 MHz, DMSO-d₆) δ8.08 (d, J=2.4 Hz, 1H), 7.85 (dd, J=8.4, 2.5 Hz, 1H), 7.31 (d, J=7.8 Hz, 1H), 7.08 (d, J=8.4 Hz, 1H), 6.97-6.91 (m, 2H), 6.88 (t, J=4.8 Hz, 1H), 5.10 (s, 1H), 4.36 (q, J=7.1, 5.5 Hz, 2H), 3.80-3.67 (m, 2H), 3.62 (td, J=8.6, 8.0, 3.9 Hz, 1H), 3.54 (d, J=11.4 Hz, 1H), 2.95 (s, 3H), 2.79 (d, J=4.6 Hz, 3H), 1.92-1.74 (m, 2H), 1.33 (s, 3H).

### Example 17

### (S)-2-(3-((5-(((5-(Hydroxymethyl)-4-methyl-6-carbonyl-5,6,9,10-tetrahydro-4H,8H-pyrido[3,2,1-de]pteridin-2-yl)amino)methyl)pyridin-2-yl)oxy)-6-(trifluoromethyl) pyridin-2-yl)acetonitrile

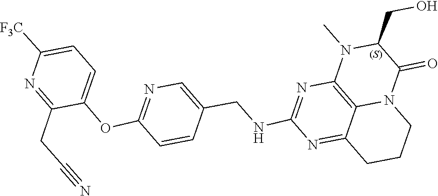

Example 17 was prepared by the following steps:

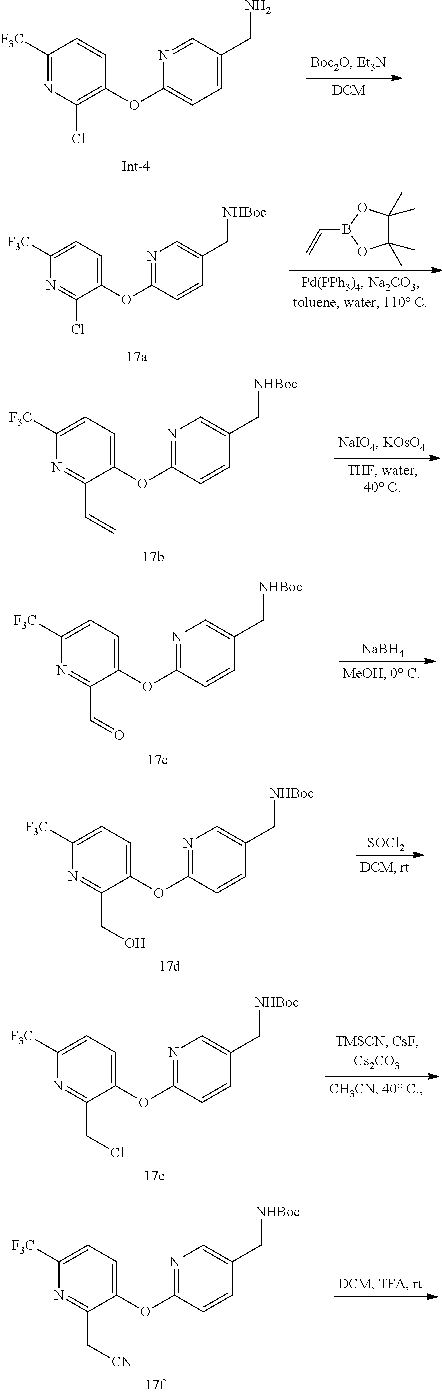

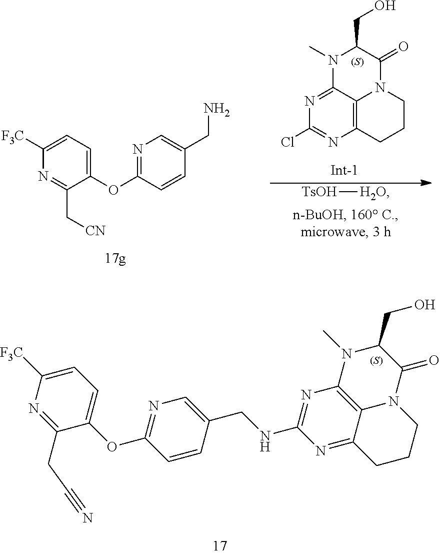

Step 1: Add di-tert-butyl carbonic anhydride (1.75 g, 8.02 mmol) dropwise to a solution of Int-4 (2.03 g, 6.68 mmol) and triethylamine (1.39 mL, 10.02 mmol) in dichloromethane (40 mL) under ice-water bath conditions. The reaction mixture was stirred at room temperature for 16 hours, and then water was added to quench the reaction. The product was extracted with dichloromethane, and the organic phases were combined and washed with saturated brine, dried over anhydrous sodium sulfate, and the organic phase was concentrated and purified by silica gel column chromatography (petroleum ether:ethyl acetate=3:1) to obtain a white solid 17a (1.95 g, yield 72%). ESI-MS (m/z): 403.9 [M+H]⁺.

Step 2: Compound 17a (675 mg, 1.67 mmol), vinylboronic acid pinacol ester (1.29 g, 8.36 mmol), tetrakistriphenylphosphine palladium (194 mg, 0.17 mol), and sodium carbonate (354 mg, 3.34 mmol) were added to a mixed solution of water (3 mL) and 1,4-dioxane (17 mL). After nitrogen was replaced in the reaction system, the reaction was stirred at 120° C. under nitrogen protection for 16 hours, and the reaction was completed as monitored by LCMS. The solvent was distilled off under reduced pressure, and water and ethyl acetate were added for extraction. The organic phases were combined and washed with saturated brine and dried over anhydrous sodium sulfate. The organic phase was concentrated and purified by silica gel column chromatography (petroleum ether:ethyl acetate=3:1) to obtain a white solid 17b (609 mg, yield 92%). ESI-MS (m/z): 396.4 [M+H]⁺.

Step 3: Sodium periodate (907 mg, 4.24 mmol) was added to a mixed solution of 17b (559 mg, 1.41 mmol) in tetrahydrofuran (12 mL) and water (3 mL). The reaction solution was stirred at room temperature for one minute, and then potassium osmate (41 mg, 0.14 mmol) was added thereto. The reaction solution was stirred at 40° C. for 2 h, and the reaction was completed as monitored by LCMS. Water and ethyl acetate were added for extraction, and the organic phases were combined and washed with aqueous sodium thiosulfate solution and saturated brine in sequence, and dried over anhydrous sodium sulfate. The organic phase was concentrated to obtain a crude product 17c. ESI-MS (m/z): 398.3 [M+H]⁺.

Step 4: Sodium borohydride (80 mg, 2.12 mmol) was added to a solution of the crude product 17c in methanol (15 mL) under ice-water bath conditions. The reaction solution was stirred at this temperature for two hours. After the reaction was completed as monitored by LCMS, water and ethyl acetate were added for extraction. The organic phases were combined and washed with saturated brine and dried over anhydrous sodium sulfate. The organic phase was concentrated and purified by silica gel column chromatography (petroleum ether:ethyl acetate=2:1) to obtain a white solid 17d (359 mg, two-step yield 64%). ESI-MS (m/z): 400.3 [M+H]⁺.

Step 5: Thionyl chloride (0.13 mL, 1.80 mmol) was added dropwise to a solution of 17d (359 mg, 0.90 mmol) in dichloromethane (14 mL). The reaction solution was stirred at room temperature for 2 hours, and the reaction was completed as monitored by LCMS. The reaction solution was concentrated by distillation under reduced pressure to obtain a crude product 17e. ESI-MS (m/z): 418.2 [M+H]⁺.

Step 6: Trimethylsilyl cyanide (178 mg, 1.80 mmol) was added dropwise to a solution of cesium fluoride (119 mg, 1.80 mmol) in acetonitrile (8 mL) at 10° C. After the reaction solution was stirred for 30 minutes, cesium carbonate (586 mg, 1.80 mmol) and the crude product 17e were added thereto, and the reaction solution was heated to 40° C. and stirred at this temperature for 16 hours. After the reaction was completed as monitored by LCMS, water and ethyl acetate were added for extraction. The organic phases were combined and washed with saturated br ine, dried over anhydrous sodium sulfate, and the organic phase was concentrated to obtain a crude product 17f. ESI-MS (m/z): 409.3 [M+H]⁺.

Step 7: Trifluoroacetic acid (2 mL) was added dropwise to a solution of the above crude product 17f in dichloromethane (8 mL). The reaction solution was stirred at room temperature for 1.5 hours, and the reaction was completed as monitored by LCMS. The reaction solution was concentrated by distillation under reduced pressure, and aqueous sodium hydroxide solution was added to pH 8. The mixture was extracted with ethyl acetate. The organic phases were combined and washed with saturated brine, dried over anhydrous sodium sulfate, and the organic phase was concentrated to obtain a crude product 17 g. ESI-MS (m/z): 309.3 [M+H]⁺.

Step 8: Dissolve compound Int-1 (50 mg, 0.19 mmol) in n-butanol (2 mL), add compound Int-17g (69 mg, crude product) and p-toluenesulfonic acid monohydrate (7 mg, 37 umol), and stir the reaction solution at 160° C. for 3 hours under microwave conditions. The reaction was complete as monitored by LCMS. The reaction solution was purified by reverse phase preparative HPLC to obtain white solid 17 (20 mg, four-step reaction yield 4%). ESI-MS (m/z): 541.2 [M+H]f; ¹H NMR (500 MHz, DMSO-d₆) δ 8.15 (d, J=16.8 Hz, 1H), 8.01-7.87 (m, 3H), 7.20 (d, J=8.4 Hz, 1H), 6.97 (t, J=6.4 Hz, 1H), 4.99 (s, 1H), 4.42 (dd, J=15.1, 6.5 Hz, 1H), 4.34 (dd, J=15.2, 6.2 Hz, 1H), 4.29 (s, 2H), 4.11-3.98 (m, 2H), 3.77-3.65 (m, 2H), 3.32-3.27 (m, OH), 2.96 (s, 3H), 2.50-2.44 (m, 2H), 1.97-1.84 (m, 1H), 1.83-1.68 (m, 1H).

### Example 18

### (S)-2-(((6-((2-fluoro-6-(trifluoromethyl)pyridin-3-yl)oxy)pyridin-3-yl)methyl)amino)-5-(hydroxymethyl)-5-methyl-4-(methyl-d3)-4,5,9,10-tetrahydro-6H,8H-pyrido[3,2,1-de]pteridin-6-one

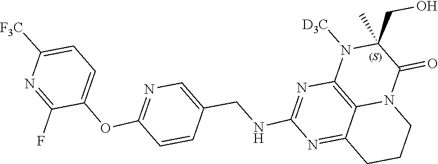

Example 18 was prepared by the following steps:

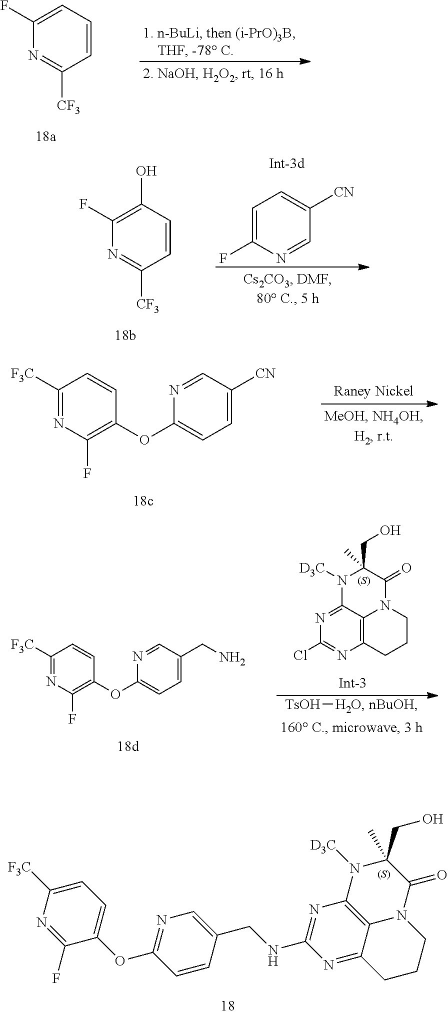

Step 1: Dissolve compound 18a (300 mg, 1.82 mmol) in anhydrous tetrahydrofuran (5 mL). Under nitrogen protection, cool to −78° C. and slowly add n-butyl lithium (1.19 mL, 1.91 mmol). After one hour, start adding triisopropyl borate (0.63 mL, 2.73 mmol). After two hours, the reaction was completed as monitored by LCMS. Water was slowly added dropwise to quench the reaction. When the system was heated to 0° C., sodium hydroxide aqueous solution (4 M, 1.36 mL, 5.45 mmol) and hydrogen peroxide (1 mL) were added dropwise and the reaction was continued at room temperature for 16 h. The reaction solution was acidified with hydrochloric acid (2 M), extracted three times with dichloromethane and concentrated. The residue was purified by silica gel column chromatography (dichloromethane:methanol=10:1) to give a yellow solid 18b (280 mg, yield 85%). MS (m/z): 180.5 [M−H]⁻.

Step 2: Compound 18b (280 mg, 1.55 mmol), Int-3d (226 mg, 1.86 mmol) and cesium carbonate (1.01 g, 3.09 mmol) were added to acetonitrile (10 mL) and reacted at room temperature for 16 hours. The reaction was completed as monitored by LCMS. Acetonitrile was distilled off under reduced pressure, and water and ethyl acetate were added for extraction. The organic phases were combined and washed with saturated brine and dried over anhydrous sodium sulfate. The organic phase was concentrated and purified by silica gel column chromatography (petroleum ether:ethyl acetate=5:1) to give a yellow oily liquid 18c (270 mg, yield 61%). ESI-MS (m/z): 284.4 [M+H]⁺.

Step 3: Compound 18c (270 mg, 0.95 mmol) was dissolved in methanol (30 mL). Ammonia water (3.5 mL) and Raney nickel (3.5 mL, aqueous suspension) were added to the reaction system in sequence. The hydrogen was replaced by a hydrogen balloon and the mixture was reacted under a hydrogen atmosphere at room temperature for 3 hours. The reaction was completed as monitored by LCMS. The reaction solution was diluted with methanol, filtered, and the filtrate was concentrated to obtain brown oily liquid 18d (270 mg, yield 98%). ESI-MS (m/z): 271.5 [M+H]⁺.

Step 4: Compound 18d (65.33 mg, 0.23 mmol) was dissolved in n-butanol (2 mL), and compound Int-3 (50 mg, 0.17 mmol) and p-toluenesulfonic acid monohydrate (3.32 mg, 17 umol) were added. The reaction solution was stirred at 160° C. for 3 hours under microwave conditions. The reaction solution was cooled to room temperature and concentrated under reduced pressure. The residue was purified by reverse phase preparative HPLC to obtain a white solid 18 (5.27 mg, yield 5%). ESI-MS (m/z): 536.5 [M+H]⁺; ¹H NMR (500 MHz, DMSO-d6) δ 8.20 (t, J=8.6 Hz, 1H), 8.09 (d, J=2.1 Hz, 1H), 8.00 (d, J=8.0 Hz, 1H), 7.92 (dd, J=8.5, 2.1 Hz, 1H), 7.23 (d, J=8.5 Hz, 1H), 6.98-6.89 (m, 1H), 5.07 (t, J=5.7 Hz, 1H), 4.43-4.33 (m, 2H), 3.79-3.68 (m, 2H), 3.66-3.60 (m, 1H), 3.59-3.52 (m, 1H), 2.50-2.46 (m, 2H), 1.89-1.75 (m, 2H), 1.33 (s, 3H).

According to the synthetic routes and the synthetic methods of the intermediates described in the above examples, the following examples can be obtained.

| Ex- | | |
|---|---|---|
| ample | structure | analyze data |
| 19 | 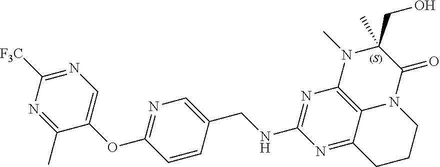 | ESI-MS (m/z): 531.5 [M + H]⁺; ¹H NMR (500 MHz, DMSO-d6) δ 8.88 (s, 1H), 8.09 (d, J = 2.4 Hz, 1H), 7.93 (dd, J = 8.5, 2.4 Hz, 1H), 7.24 (d, J = 8.4 Hz, 1H), 6.96-6.90 (m, 1H), 5.10-5.04 (m, 1H), 4.40-4.35 (m, 2H), 3.77-3.69 (m, 2H), 3.65-3.60 (m, 1H), 3.58-3.53 (m, 1H), 2.96 (s, 3H), 2.49-2.47 (m, 2H), 2.44 (s, 3H), 1.86-1.79 (m, 2H), 1.34 (s, 3H). |
| 20 | 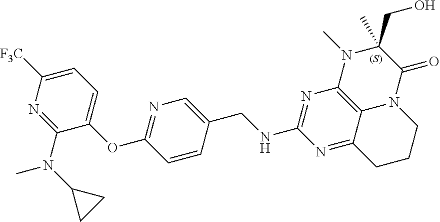 | ESI-MS (m/z): 585.5 [M + H]⁺; ¹H NMR (500 MHz, DMSO-d6) δ 8.05 (d, J = 2.3 Hz, 1H), 7.82 (dd, J = 8.5, 2.4 Hz, 1H), 7.52 (d, J = 7.9 Hz, 1H), 7.23 (d, J = 8.0 Hz, 1H), 7.04 (d, J = 8.3 Hz, 1H), 6.88 (t, J = 6.3 Hz, 1H), 5.06 (s, 1H), 4.51-4.23 (m, 2H), 3.76-3.66 (m, 2H), 3.62 (ddd, J = 12.6, 7.4, 4.3 Hz, 1H), 3.54 (d, J = 11.3 Hz, 1H), 2.95 (s, 3H), 2.91 (s, 3H), 2.64 (dp, J = 10.3, 3.3 Hz, 1H), 2.47 (d, J = 6.3 Hz, 2H), 1.91-1.74 (m, 2H), 1.33 (s, 3H), 0.63-0.52 (m, 2H), 0.42-0.26 (m, 2H). |
| 21 | 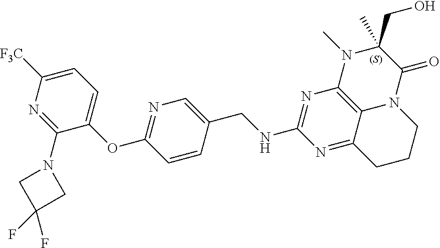 | ESI-MS (m/z): 606.4 [M + H]⁺; ¹H NMR (500 MHz, DMSO-d6) δ 8.08 (d, J = 2.3 Hz, 1H), 7.86 (dd, J = 8.5, 2.4 Hz, 1H), 7.61 (d, J = 8.0 Hz, 1H), 7.31 (d, J = 8.0 Hz, 1H), 7.12 (d, J = 8.4 Hz, 1H), 6.91 (s, 1H), 5.08 (t, J = 5.5 Hz, 1H), 4.45-4.40 (m, 4H), 4.38-4.33 (m, 2H), 3.75-3.65 (m, 2H), 3.63-3.57 (m, 1H), 3.55-3.50 (m, 1H), 2.93 (s, 3H), 2.50-2.42 (m, 2H), 1.85-1.75 (m, 2H), 1.31 (s, 3H). |
| 22 | 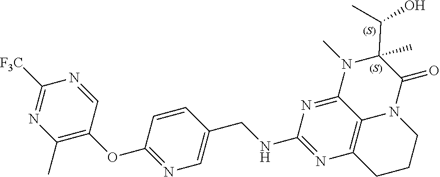 | ESI-MS (m/z): 544.3 [M + H]⁺; ¹H NMR (500 MHz, DMSO-d6) δ 8.86 (s, 1H), 8.06 (d, J = 2.2 Hz, 1H), 7.93-7.89 (m, 1H), 7.23 (d, J = 8.4 Hz, 1H), 7.01 (s, 1H), 4.86 (d, J = 5.6 Hz, 1H), 4.44-4.29 (m, 2H), 4.01 (d, J = 12.6 Hz, 1H), 3.80-3.70 (m, 1H), 3.31-3.21 (m, 2H), 3.03 (s, 3H), 2.41 (s, 3H), 1.87-1.80 (m, 2H), 1.51 (s, 3H), 0.94 (d, J = 6.5 Hz, 3H). |
| 23 | 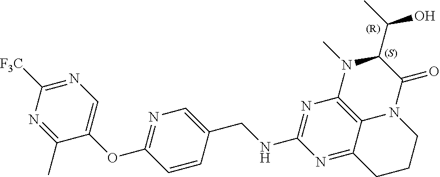 | ESI-MS (m/z): 531.5 [M + H]⁺; ¹H NMR (500 MHz, DMSO-d6) δ 8.87 (s, 1H), 8.08 (d, J = 2.3 Hz, 1H), 7.93 (dd, J = 8.4, 2.3 Hz, 1H), 7.23 (d, J = 8.4 Hz, 1H), 7.07-6.96 (m, 1H), 4.92 (d, J = 5.5 Hz, 1H), 4.46-4.39 (m, 1H), 4.36-4.29 (m, 1H), 4.16-4.08 (m, 1H), 3.92-3.84 (m, 2H), 3.17-3.11 (m, 1H), 3.07 (s, 3H), 2.51-2.45 (m, 2H), 2.43 (s, 3H), 1.97-1.90 (m, 1H), 1.80-1.69 (m, 1H), 1.09 (d, J = 6.2 Hz, 3H). |
| 24 | 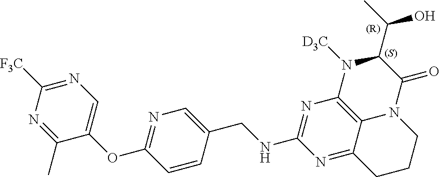 | ESI-MS (m/z): 534.5 [M + H]⁺; ¹H NMR (500 MHz, DMSO-d6) δ 8.86 (s, 1H), 8.07 (d, J = 2.2 Hz, 1H), 7.92 (dd, J = 8.4, 2.2 Hz, 1H), 7.22 (d, J = 8.5 Hz, 1H), 7.10-6.94 (m, 1H), 4.91 (d, J = 5.5 Hz, 1H), 4.45-4.38 (m, 1H), 4.35-4.27 (m, 1H), 4.14-4.06 (m, 1H), 3.95- 3.83 (m, 2H), 3.17-3.08 (m, 1H), 2.51-2.45 (m, 2H), 2.42 (s, 3H), 1.96-1.88 (m, 1H), 1.79-1.68 (m, 1H), 1.08 (d, J = 6.3 Hz, 3H). |
| 25 | 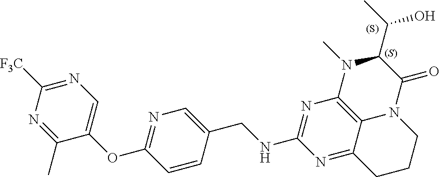 | ESI-MS (m/z): 531.5 [M + H]⁺; ¹H NMR (500 MHz, DMSO-d6) δ 8.86 (s, 1H), 8.07 (s, 1H), 7.95-7.85 (m, 1H), 7.22 (d, J = 8.4 Hz, 1H), 6.96 (s, 1H), 5.01 (d, J = 4.7 Hz, 1H), 4.44-4.31 (m, 2H), 4.19-4.03 (m, 2H), 4.02 (s, 1H), 3.30 (s, 2H), 3.00 (s, 3H), 2.47-2.44 (m, 2H), 2.42 (s, 3H), 1.85-1.76 (m, 2H), 0.99 (d, J = 6.4 Hz, 3H). |
| 26 | 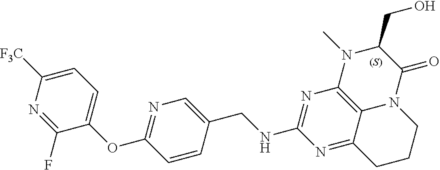 | ESI-MS (m/z): 520.4 [M + H]⁺; ¹H NMR (500 MHz, DMSO-d6) δ 8.18 (t, J = 8.6 Hz, 1H), 8.07 (d, J = 2.3 Hz, 1H), 7.98 (d, J = 8.0 Hz, 1H), 7.90 (dd, J = 8.4, 2.3 Hz, 1H), 7.21 (d, J = 8.5 Hz, 1H), 6.93 (t, J = 6.5 Hz, 1H), 4.97 (t, J = 5.6 Hz, 1H), 4.45-4.28 (m, 2H), 4.11-3.93 (m, 2H), 3.77-3.64 (m, 2H), 3.31-3.27 (m, 1H), 2.95 (s, 3H), 2.47-2.43 (m, 2H), 1.94-1.83 (m, 1H), 1.82-1.71 (m, 1H). |
| 27 | 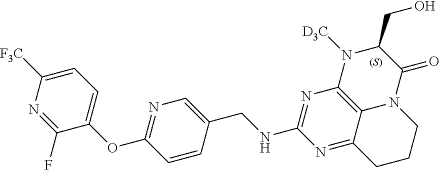 | ESI-MS (m/z): 523.5 [M + H]⁺; ¹H NMR (500 MHz, DMSO-d6) δ 8.24 (s, 1H), 8.18 (t, J = 8.6 Hz, 1H), 8.07 (d, J = 2.2 Hz, 1H), 7.98 (d, J = 8.1 Hz, 1H), 7.94-7.87 (m, 1H), 7.21 (d, J = 8.4 Hz, 1H), 6.92 (t, J = 6.1 Hz, 1H), 4.96 (s, 1H), 4.48-4.28 (m, 2H), 4.08-3.94 (m, 2H), 3.75-3.65 (m, 2H), 2.46-2.40 (m, 2H), 1.95-1.84 (m, 1H), 1.80-1.71 (m, 1H). |
| 28 | 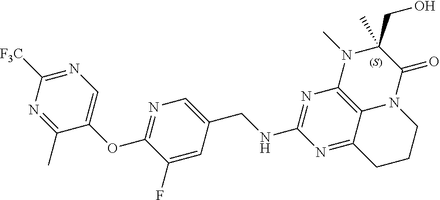 | ESI-MS (m/z): 549.5 [M + H]⁺; ¹H NMR (500 MHz, DMSO-d6) δ 8.98 (s, 1H), 8.01-7.84 (m, 2H), 6.93 (s, 1H), 5.05 (t, J = 5.7 Hz, 1H), 4.52-4.34 (m, 2H), 3.79-3.67 (m, 2H), 3.66-3.59 (m, 1H), 3.60-3.53 (m, 1H), 2.96 (s, 3H), 2.55-2.45 (m, 5H), 1.90-1.77 (m, 2H), 1.34 (s, 3H). |

### Biological Screening and Results of Wnt Pathway Inhibitors

### Test Example 1: Construction of Colo205-LUC-TCF/LEF-M1 Reporter Cell Line

Colo205 cell line (Cell Bank of Chinese Academy of Sciences, Cat #TCHul02) was purchased from the Cell Bank of Chinese Academy of Sciences. After expansion and subculture, during the exponential growth phase, luciferase reporter plasmid (Promega) driven by TCF/LEF transcription factor was transfected by lipo3000 liposome transfection. The plasmid carries a resistance gene for resistance screening. Transfection was carried out in 10 cm culture dishes using conventional complete medium without resistance. After 2 days, the medium with resistance was replaced, and the culture was continued. After that, the medium with resistance was replaced every 2 days, and the suspended cells were discarded. The original medium was centrifuged to remove cells and debris and retained as an adaptive medium. When the cells covered the culture dish, the cells were digested, counted, and passaged in a 96-well plate, so that the average number of cells contained in each well was 1.5/well, and the adaptive medium was used for passage. The rest of the cells were frozen. After passage, culture for 4 hours to allow the cells to adhere to the wall, and then observe the number of cells in each well under a microscope. Wells with only 1 cell per well were labeled as monoclonal wells. Afterwards, normal culture was performed, and the culture medium was replaced every 2 days, and observed. There are wells where the monoclonal cells continue to grow in the early stage, and they are labeled twice, and can be replaced with normal medium with resistance. When a monoclonal well was overgrown with a 96-well plate, it is digested and passaged to a 24-well culture plate. After the 24-well plate is overgrown, it is passaged to a 96-well plate and a 6-well plate. The cells in a 96-well plate are at least 6 wells, of which 3 wells were added with known Wnt inhibitors, and the other 3 wells were not treated. After 24 hours, the cells in the 96-well plate were added with a fluorescence detection reagent to detect the fluorescence intensity. Cell lines with fluorescent expression when not treated and decreased fluorescent light after inhibition were selected and further cultured. The Colo205-LUC-TCF/LEF-M1 cell line is one of the cell lines screened above. Its growth curve, cell shape, and cell growth state are similar to those of the original Colo205 cells, and the ratio of the fluorescent signals of the inhibitor-treated and untreated cells is the largest among all cell lines, and the ratio can reach 4-5 times when inhibited at 4 hours, which was completely suitable for the screening of Wnt inhibitors in the later stage.

### Test Example 2: Detection of Compound's Inhibitory Ability on Colo205-LUC-TCF/LEF M1 Reporter Cell Line

The Colo205-LUC-TCF/LEF M1 cell line is a reporter tool cell stably transfected with the pGL4.49-LUC2-TCF/LEF vector. The β-catenin Wnt pathway is continuously activated. After adding the inhibitor, the Wnt pathway is inhibited. The expression of firefly luciferase regulated by the TCF/LEF cis-element decreased, and after adding the detection substrate, the detected light signal decreased accordingly, so as to detect the inhibitory effect of the compound.

To a 96-well cell culture plate, add 100 uL of the compound with a maximum concentration of 20 uM to each well, and make a 3-fold serial dilution of the compound concentration. Then 10,000 colo205 cells stably transfected with the reporter gene and 100 uL medium were inoculated into each well, and corresponding positive and negative control wells were made at the same time. Put the cells in a 5% CO2 incubator and incubate at 37° C. for 4 hours. After 4 hours, remove the culture medium, add 100 μL of reagent (Promega) containing the corresponding firefly luciferase substrate to each well, and measure the activity of luciferase reporter gene. Luminescence intensity was read with SpectraMax in full wavelength mode. The light signal intensity of cells treated only with DMSO was used as a positive control, and the light signal intensity of wells without cells was used as a negative control, and the concentration of IC50 of each compound was calculated. Colo 205 reporter gene detection data are summarized in Table 1.

TABLE 1
IC₅₀ values of compounds for inhibition
of Colo205-LUC-TCF/LEF reporter gene

| Com- | Colo205-LUC-TCF/LEF | Com- | Colo205-LUC-TCF/LEF |
|---|---|---|---|
| pound | reporter IC₅₀ (nM) | pound | reporter IC₅₀ (nM) |
| 1 | 15.54 | 2 | 22.47 |
| 3 | 15.64 | 4 | 66.31 |
| 5 | 50.91 | 6 | 65.63 |
| 7 | 10.66 | 8 | 32.04 |
| 9 | 13.75 | 10 | 3.89 |
| 11 | 14.00 | 12 | 9.09 |
| 13 | 6.86 | 14 | 35.23 |
| 15 | <0.1 | 16 | 21.52 |
| 17 | 263.98 | 18 | 29.90 |
| 19 | 34.77 | 20 | 1.46 |
| 21 | 4.94 | 22 | 90.04 |
| 23 | 64.65 | 24 | 47.97 |
| 25 | 69.43 | 26 | 43.84 |
| 27 | 30.65 | 28 | 27.40 |

### Test Example 3: Proliferation Inhibition Test of Compounds on Wnt Mutant Cell Lines (Colo205, H929, HepG2 and DU4475) and Non-Wnt Mutant Cell Lines (RKO)

The cell lines used in the experiment are Colo205, H929, HepG2, and DU4475, which have sustained activation of the Wnt pathway and whose proliferation is Wnt pathway-dependent. On the other hand, RKO cell line, in which normally the Wnt pathway is not activated and whose proliferation is not Wnt pathway-dependent, was used as a control cell line. This was done to determine whether the inhibitory effect of the compound of the present disclosure on Wnt-dependent proliferation is due to other nonspecific toxicity.

Colo205, H929, DU4475, HepG2 and RKO cell lines cultured in their respective culture media were treated in the logarithmic growth phase, and the cells were collected and prepared into a uniform cell suspension of known concentration, and then added to a 96-well cell culture plate so that each well contained 1000-4000 cells. Place in a 5% CO2 cell culture incubator and culture at 37° C. for 20-24 h. On the second day, add the completely dissolved, 3-fold gradient dilution compound to each cell culture well to make the final maximum concentration in the cell culture well 10 uM, and continue to culture for 96 h. This test uses Promega's cell activity detection test for detection. The more the cells proliferate, the stronger the final signal intensity. The detection instrument is SpectraMax, full wavelength mode. The wells with only DMSO added were used as positive control wells, and the wells without cells inoculated were used as negative control wells. The IC50 values of each compound for the inhibition of proliferation of cells with continuous Wnt activation or proliferation dependence, as well as the IC50 values for the inhibition of proliferation of cells with inactive Wnt or proliferation-independence were calculated to evaluate the inhibitory effect of the compound on the Wnt pathway and the toxic effect on normal cells (Table 2).

TABLE 2
IC₅₀ values of compounds for inhibition
of proliferation of Wnt mutant cell lines

| | Antiproliferation IC50 (nM) | | | | |
|---|---|---|---|---|---|
| Compound | Colo205 | DU4475 | H929 | HepG2 | RKO |
| 1 | 7.81 | 3.17 | 40.31 | 1.45 | >10000 |
| 2 | 8.67 | 5.05 | 47.72 | 1.68 | >10000 |
| 3 | 4.48 | 3.06 | 20.04 | 2.17 | >10000 |
| 4 | 42.59 | 25.75 | 277.77 | 89.74 | >10000 |
| 5 | 20.56 | 14.37 | 160.44 | 33.52 | >10000 |
| 6 | 9.05 | 47.39 | 302.67 | 109.23 | >10000 |
| 7 | 5.54 | 3.59 | 52.25 | 15.55 | >10000 |
| 8 | 24.17 | 9.60 | 86.86 | 35.08 | >10000 |
| 9 | 8.43 | 0.46 | 36.39 | 11.29 | >10000 |
| 10 | 2.34 | <0.5 | 17.97 | 3.54 | >10000 |
| 11 | 11.85 | 1.47 | 83.14 | 32.79 | >10000 |
| 12 | 6.52 | 0.82 | 31.26 | 12.13 | >10000 |
| 13 | 11.49 | 1.58 | 54.01 | 19.14 | >10000 |
| 14 | 70.27 | ND | ND | 78.97 | >10000 |
| 15 | 1.35 | ND | ND | 1.3 | >10000 |
| 16 | <0.1 | 1.2 | 103.43 | 1.13 | >10000 |
| 17 | 178.81 | 42.94 | 598.96 | 878.88 | >10000 |
| 18 | 7.43 | 6.72 | 78.78 | 22.07 | >10000 |
| 19 | 26.61 | 14.78 | 58.28 | 31.15 | >10000 |
| 20 | <0.1 | <0.1 | 51.96 | 3.22 | >10000 |
| 21 | <0.1 | 4.83 | 254.70 | 1.52 | >10000 |
| 22 | 64.28 | 38.63 | 308.51 | 223.64 | >10000 |
| 23 | 53.66 | 16.01 | 191.72 | 37.45 | >10000 |
| 24 | 46.48 | 23.19 | 5.61 | 36.96 | >10000 |
| 25 | 64.45 | 15.95 | 97.99 | 68.02 | >10000 |
| 26 | 18.95 | 12.07 | 98.82 | 13.64 | >10000 |
| 27 | 22.80 | 24.59 | 90.15 | 38.34 | >10000 |
| 28 | 25.67 | 16.82 | 126.40 | 61.33 | >10000 |

ND = Not Tested

The above results show that the compounds of the present disclosure have significant inhibitory activity against mutant cell lines Colo205, DU4475, NCI-H929 and HepG2, but have basically no significant inhibitory activity against Hela and RKIO cell lines, which indicates that the compounds of the present disclosure have significant Wnt-dependent proliferation inhibitory effect.

1. A compound having a structure of formula I or a pharmaceutically acceptable salt, isotopic derivative, or stereoisomer thereof: 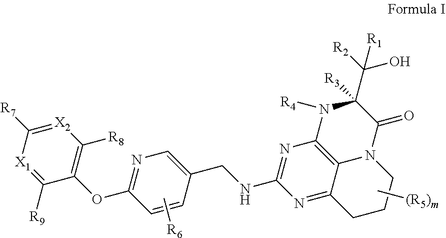
wherein, X₁ and X₂ are both N;
R₁ and R₂ each independently represent hydrogen, halogen, (C₁-C₆) alkyl, or halogenated (C₁-C₆) alkyl;
R₃ represents hydrogen, halogen, (C₁-C₆) alkyl, halogenated (C₁-C₆) alkyl;
R₄ represents hydrogen, halogen, (C₁-C₆) alkyl, halogenated (C₁-C₆) alkyl, or R₄ and R₂ form a 4-8 membered ring;
R₅ each independently represents hydrogen, halogen, (C₁-C₆) alkyl, halogenated (C₁-C₆) alkyl, (C₃-C₈) cycloalkyl, halogenated (C₃-C₈) cycloalkyl, or two R₅ connected to the same carbon atom form a 3-5 membered ring; m is 0, 1, 2 or 3;
R₆ each independently represents hydrogen, halogen, —CN, (C₁-C₆) alkyl, halogenated (C₁-C₆) alkyl;
R₇ represents hydrogen, halogen, (C₁-C₆) alkyl, halogenated (C₁-C₆) alkyl, (C₃-C₈) cycloalkyl, halogenated (C₃-C₈) cycloalkyl, —ORₐ, -halogenated ORₐ, —SRₐ, -halogenated SRₐ;
R₈ represents hydrogen, (C₁-C₆) alkyl, halogenated (C₁-C₆) alkyl;
R₉ represents halogen, (C₁-C₆) alkyl, halogenated(C₁-C₆) alkyl, (C₃-C₈) cycloalkyl, halogenated(C₃-C₈) cycloalkyl, —(C₁-C₆) alkylene CN, -halogenated (C₁-C₆) alkylene CN, —(C₃-C₈) cycloalkylene CN, -halogenated(C₃-C₅) cycloalkylene CN, —NRₐRₐ′, —(C₁-C₆) alkylene NRₐRₐ′, -halogenated(C₁-C₆) alkylene NRₐRₐ′, wherein Rₐ, Rₐ′ can form a 4-8 membered ring with the N to which they are connected,
Rₐ and Rₐ′ each independently represent hydrogen, (C₁-C₆) alkyl, halogenated (C₁-C₆) alkyl, (C₃-C₈) cycloalkyl or halogenated (C₃-C₈) cycloalkyl.

2. The compound having a structure of formula I according to claim 1 or a pharmaceutically acceptable salt, isotopic derivative, stereoisomer thereof, wherein R₄ is selected from (C₁-C₆) alkyl and halogenated (C₁-C₆) alkyl.

3. The compound having a structure of formula I according to claim 1, or a pharmaceutically acceptable salt, isotopic derivative, stereoisomer thereof, wherein R₅ is selected from hydrogen, halogen, (C₁-C₆) alkyl, halogenated (C₁-C₆) alkyl.

4. The compound having a structure of formula I according to claim 1, or a pharmaceutically acceptable salt, isotopic derivative, stereoisomer thereof, wherein R₅ is a halogen.

5. The compound having a structure of formula I according to claim 1, or a pharmaceutically acceptable salt, isotopic derivative, stereoisomer thereof, wherein R₅ is fluorine.

6. The compound having a structure of formula I according to claim 1, or a pharmaceutically acceptable salt, isotopic derivative, stereoisomer thereof, wherein R₆ is hydrogen.

7. The compound having a structure of formula I according to claim 1, or a pharmaceutically acceptable salt, isotopic derivative, or stereoisomer thereof, wherein X₁ and X₂ are both N, and R₉ is selected from (C₁-C₆) alkyl, halogenated (C₁-C₆) alkyl, (C₃-C₈) cycloalkyl, or halogenated (C₃-C₈) cycloalkyl.

8. The compound having a structure of formula I according to claim 1, or a pharmaceutically acceptable salt, isotopic derivative, stereoisomer thereof, wherein when X₁ and X₂ are both N, R₇ is not a halogen.

9. The compound having a structure of formula I according to claim 1, or a pharmaceutically acceptable salt, isotopic derivative, stereoisomer thereof, wherein R₇ is selected from (C₁-C₆) alkyl, halogenated (C₁-C₆) alkyl.

10-11. (canceled)

12. A compound having a structure of formula I according to claim 1, or a pharmaceutically acceptable salt, isotopic derivative, or stereoisomer thereof, wherein, when one of X₁ and X₂ is N and the other is CH, R₉ represents (C₁-C₆) alkyl, halogenated (C₁-C₆) alkyl, (C₃-C₈) cycloalkyl, or halogenated (C₃-C₈) cycloalkyl.

13. A compound, or a pharmaceutically acceptable salt, isotopic derivative, or stereoisomer thereof, wherein the compound has the following structure:

| Serial | |
|---|---|
| number | Compound structure |
| 1 | 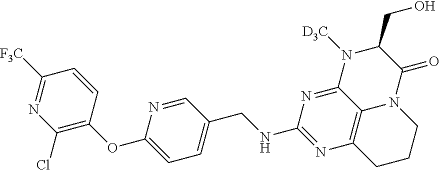 [SMILES: [2H]C([2H])([2H])N1c2nc(NCc3ccc(Oc4ccc(C(F)(F)F)nc4Cl)nc3)nc3c2N(CCC3)C(=O)[C@@H]1CO] |
| 2 | 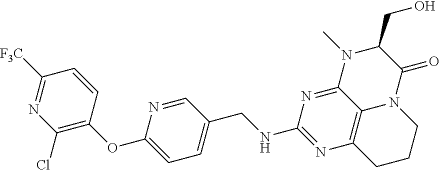 [SMILES: CN1c2nc(NCc3ccc(Oc4ccc(C(F)(F)F)nc4Cl)nc3)nc3c2N(CCC3)C(=O)[C@@H]1CO] |
| 3 | 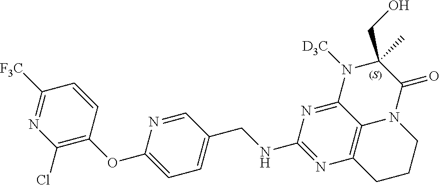 |
| 4 | 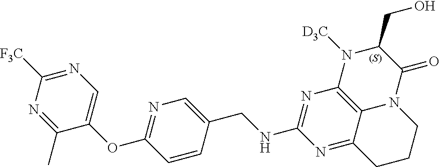 |
| 5 | 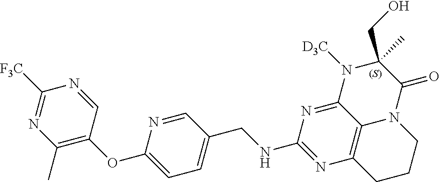 |
| 6 | 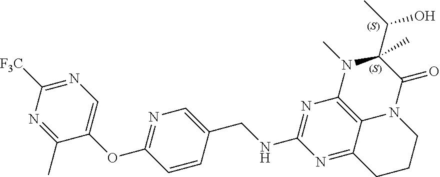 |
| 7 | 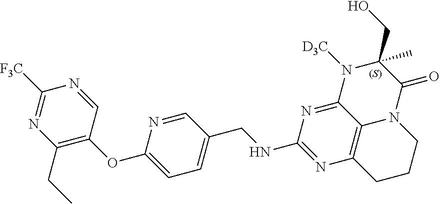 |
| 8 | 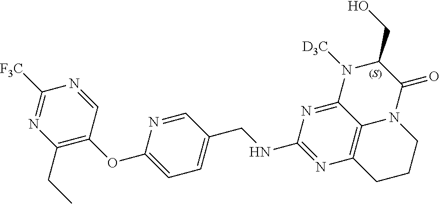 |
| 9 | 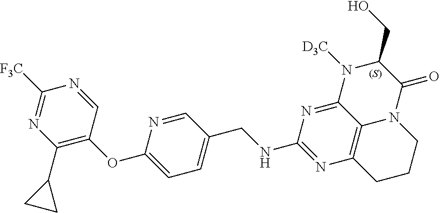 |
| 10 | 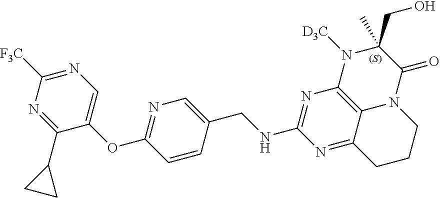 |
| 11 | 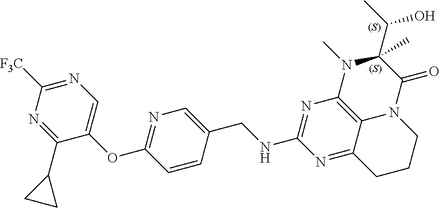 |
| 12 | 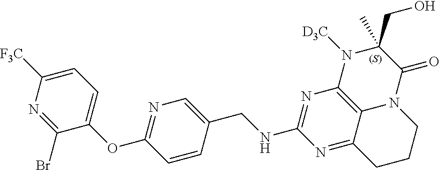 |
| 13 | 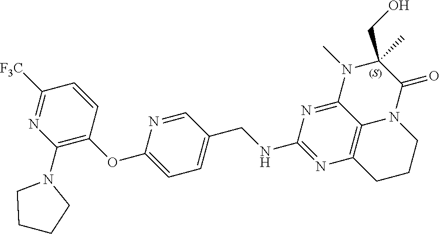 |
| 14 | 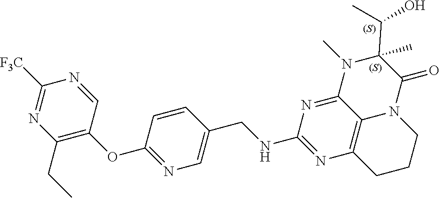 |
| 15 | 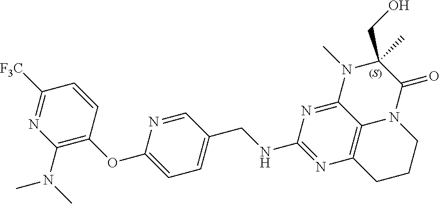 |
| 16 | 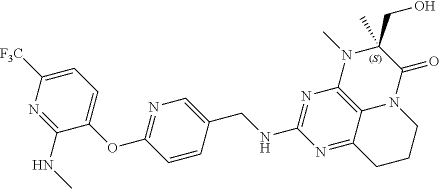 |
| 17 | 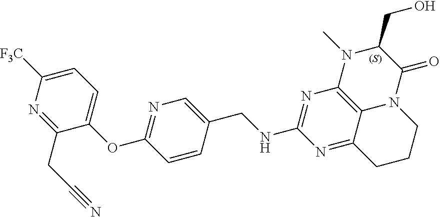 |
| 18 | 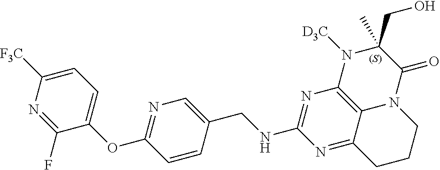 |
| 19 | 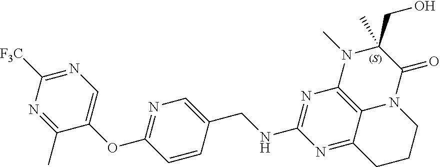 |
| 20 | 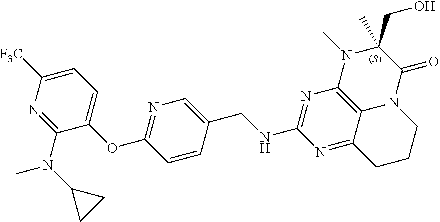 |
| 21 | 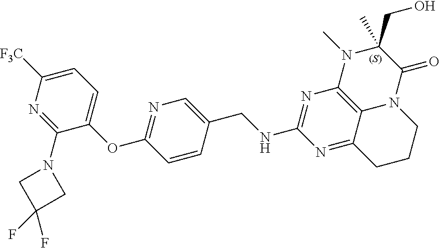 |
| 22 | 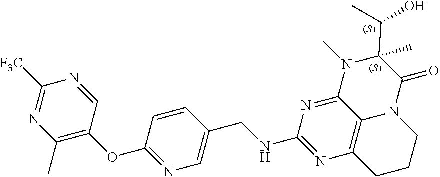 |
| 23 | 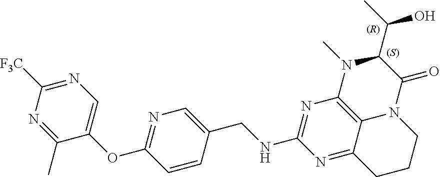 |
| 24 | 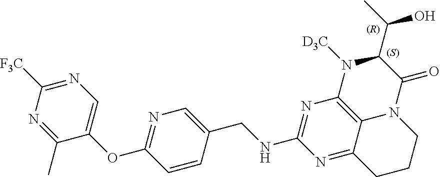 |
| 25 | 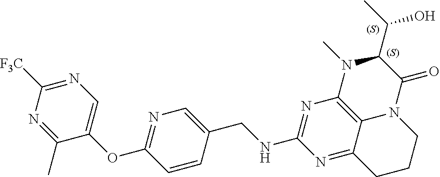 |
| 26 | 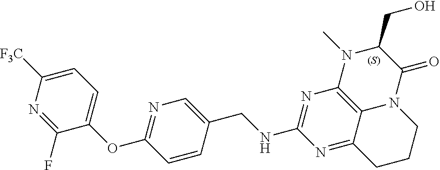 |
| 27 | 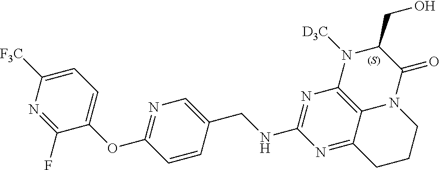 |
| 28 | 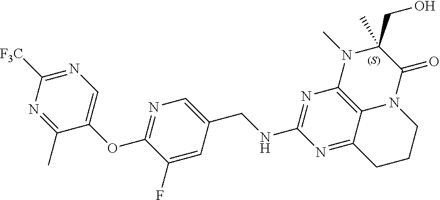 |
| 29 | 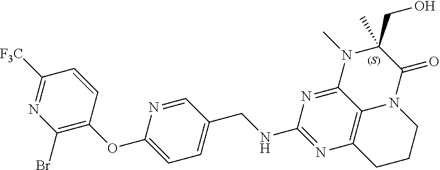 |
| 30 | 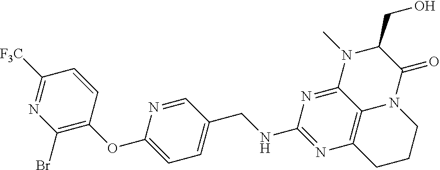 [SMILES: CN1c2nc(NCc3ccc(Oc4ccc(C(F)(F)F)nc4Br)nc3)nc3c2N(CCC3)C(=O)[C@@H]1CO] |
| 31 | 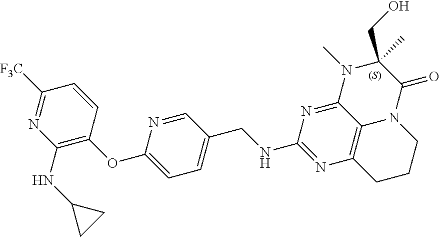 |
| 32 | 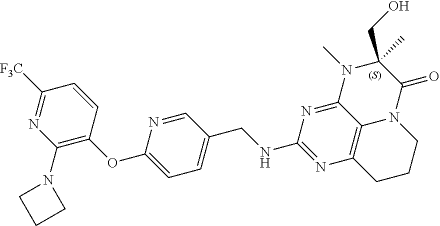 |
| 33 | 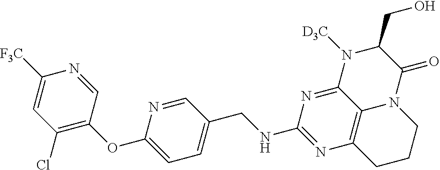 [SMILES: [2H]C([2H])([2H])N1c2nc(NCc3ccc(Oc4cnc(C(F)(F)F)cc4Cl)nc3)nc3c2N(CCC3)C(=O)[C@@H]1CO] |
| 34 | 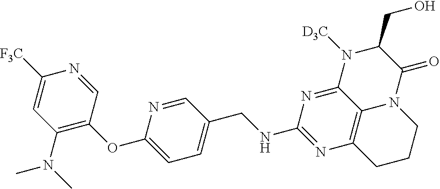 [SMILES: [2H]C([2H])([2H])N1c2nc(NCc3ccc(Oc4cnc(C(F)(F)F)cc4N(C)C)nc3)nc3c2N(CCC3)C(=O)[C@@H]1CO] |
| 35 | 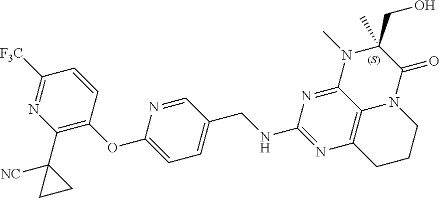 |
| 36 | 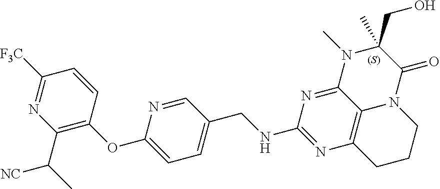 |
| 37 | 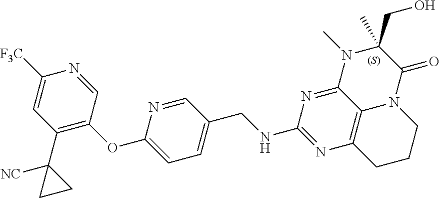 |
| 38 | 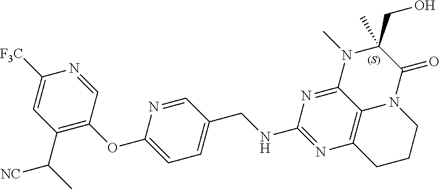 |
| 39 | 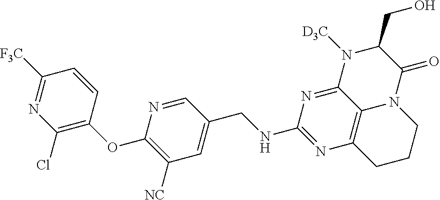 [SMILES: [2H]C([2H])([2H])N1c2nc(NCc3cnc(Oc4ccc(C(F)(F)F)nc4Cl)c(C#N)c3)nc3c2N(CCC3)C(=O)[C@@H]1CO] |
| 40 | 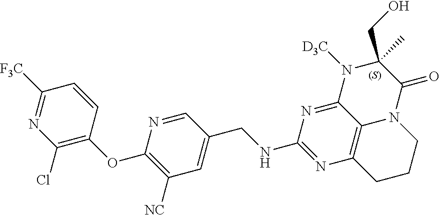 |
| 41 | 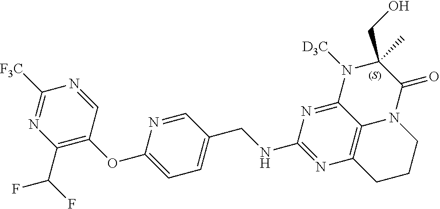 |
| 42 | 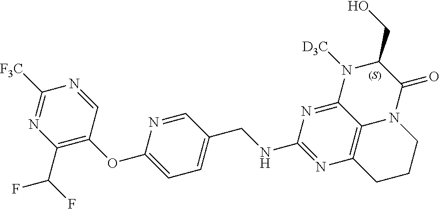 |
| 43 | 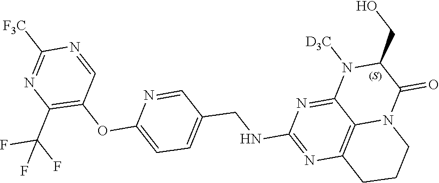 |
| 44 | 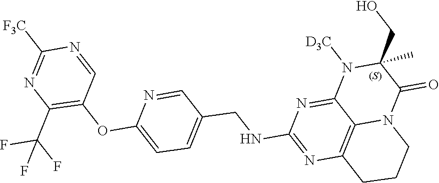 |
| 45 | 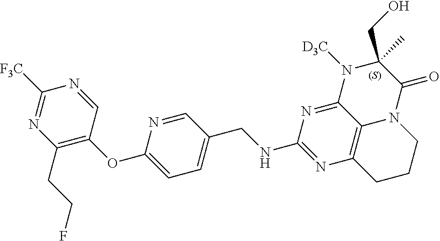 |
| 46 | 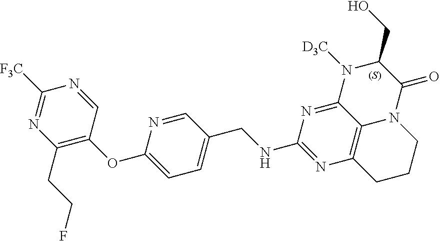 |
| 47 | 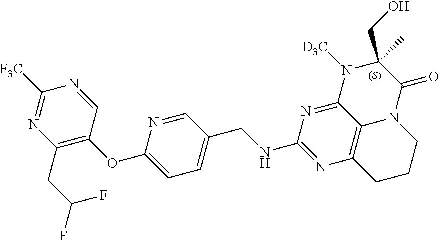 |
| 48 | 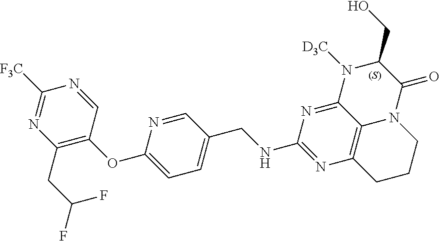 |
| 49 | 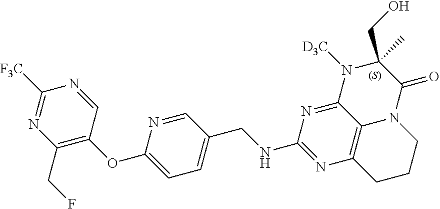 |
| 50 | 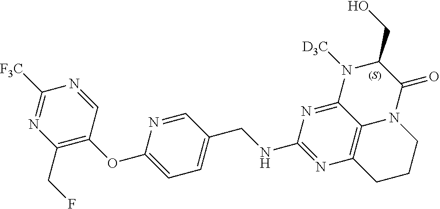 |
| 51 | 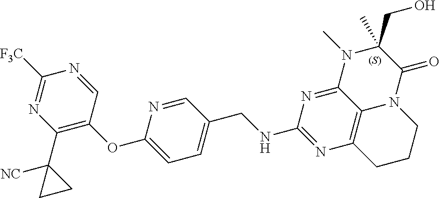 |
| 52 | 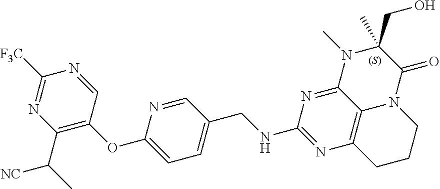 |
| 53 | 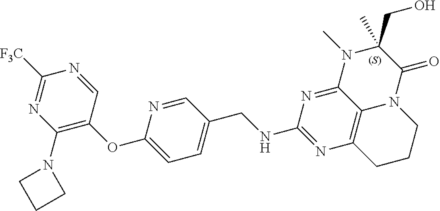 |
| 54 | 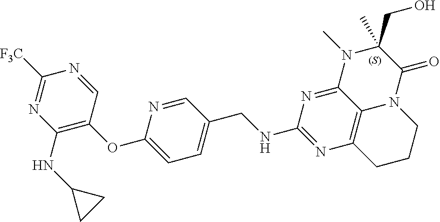 |
| 55 | 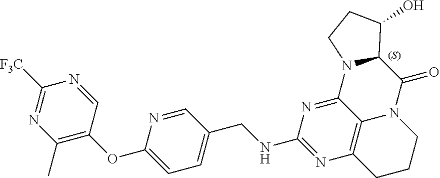 |
| 56 | 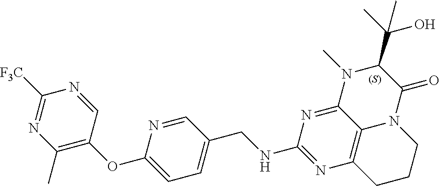 |
| 57 | 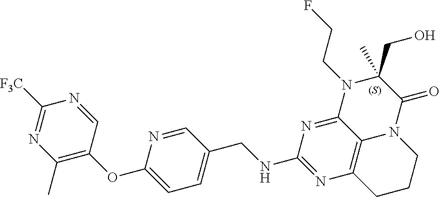 |
| 58 | 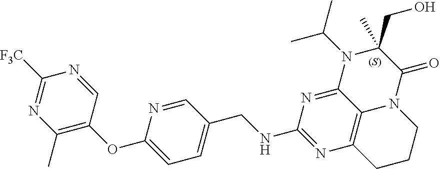 |
| 59 | 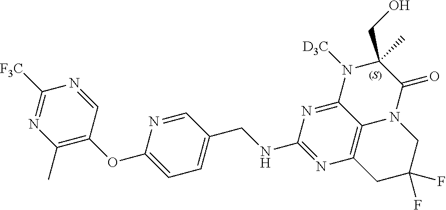 |
| 60 | 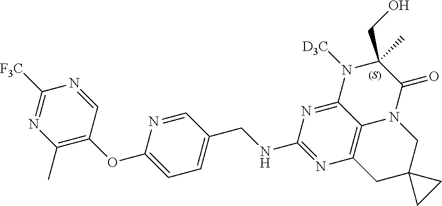 |
| 61 | 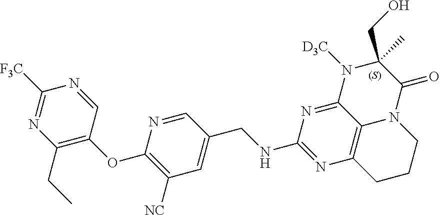 |
| 62 | 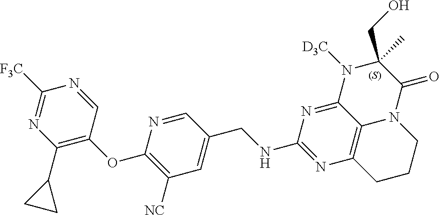 |
| 63 | 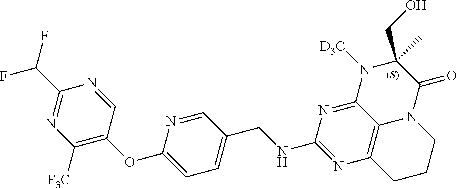 |
| 64 | 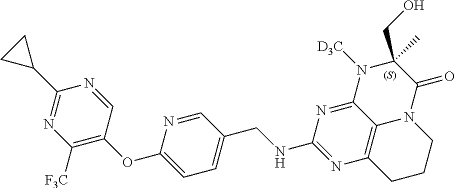 |
| 65 | 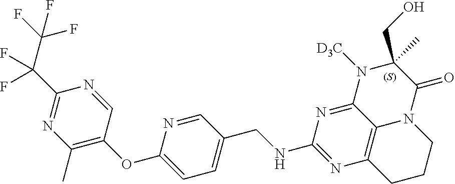 |
| 66 | 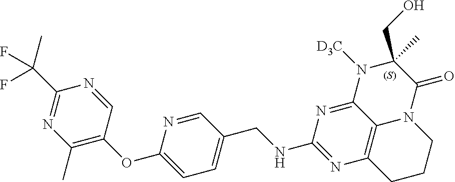 |
| 67 | 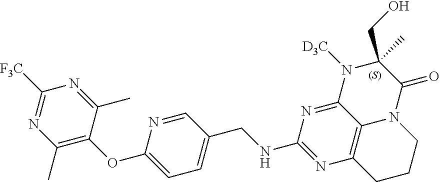 |
| 68 | 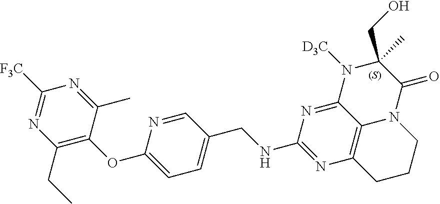 |
| 69 | 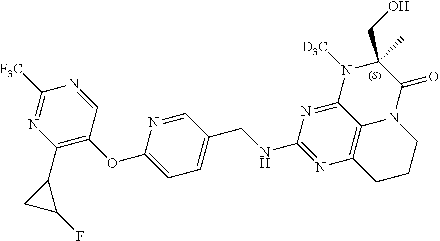 |
| 70 | 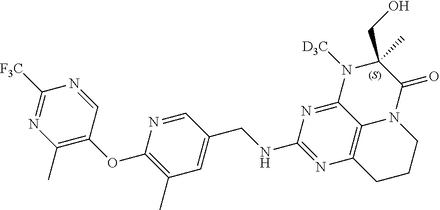 |
| 71 | 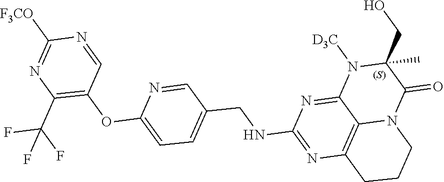 |
| 72 | 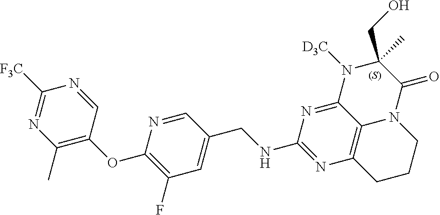 |
| 73 | 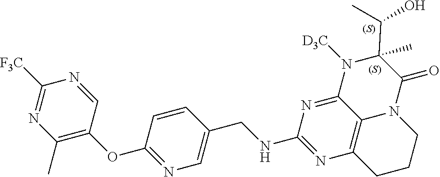 |
| 74 | 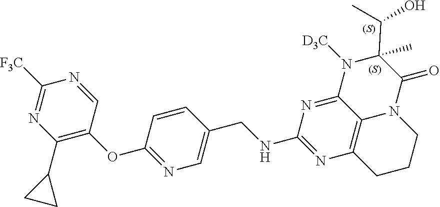 |
| 75 | 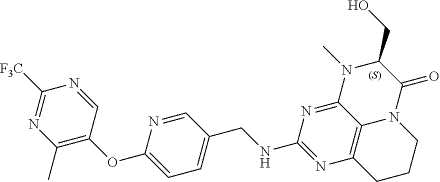 |
| 76 | 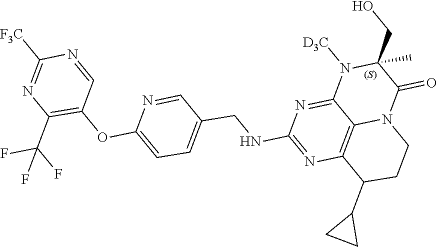 |
| 77 | 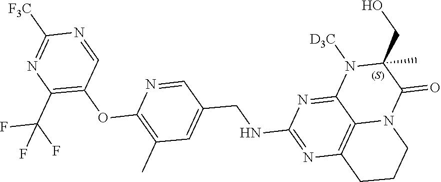 |
| 78 | 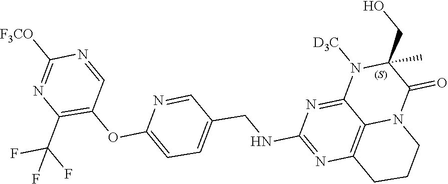 |
| 79 | 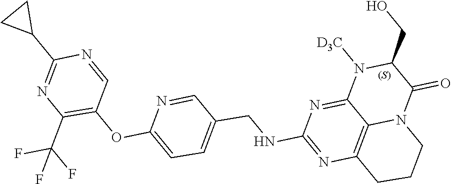 |

14. A pharmaceutical composition comprising a compound according to claim 1 or a pharmaceutically acceptable salt, isotopic derivative, stereoisomer thereof, and optionally a pharmaceutically acceptable carrier.

15. (canceled)
